(19) United States
(12) Patent Application Publication
(10) Pub. No.: US 2024/0207265 A1
(43) Pub. Date: Jun. 27, 2024

(54) TREATMENT OF CNS DISEASES WITH sGC STIMULATORS

(71) Applicant: Tisento Therapeutics Inc., Cambridge, MA (US)

(72) Inventors: Lei Jia, San Diego, CA (US); Ara Mermerian, Waltham, MA (US); Timothy Claude Barden, Gibsonia, PA (US); Thomas Wai-Ho Lee, Lexington, MA (US); Karthik Iyer, Cambridge, MA (US); Glen Robert Rennie, Somerville, MA (US); Rajesh R. Iyengar, West Newton, MA (US); Joon Jung, Newton, MA (US); Paul Allan Renhowe, Sudbury, MA (US); Susana dos Santos Correia, Cambridge, MA (US); Peter Germano, Newton, MA (US)

(21) Appl. No.: 18/287,616

(22) PCT Filed: Apr. 19, 2022
(86) PCT No.: PCT/US2022/025308

§ 371 Date: Oct. 19, 2023

Related U.S. Application Data
(60) Provisional application No. 63/229,251, filed on Aug. 4, 2021.
(60) Provisional application No. 63/177,023, filed on Apr. 20, 2021.

(51) Int. Cl. A61K 31/506; A61P 25/28

(52) U.S. Cl. CPC A61K 31/506; A61P 25/28

(57) ABSTRACT

The present disclosure relates to the use of stimulators of soluble guanylate cyclase (sGC), pharmaceutically acceptable salts thereof and pharmaceutical formulations or dosage forms comprising them, alone or in combination with one or more additional agents, for the treatment of various CNS diseases, wherein an increase in sGC stimulation, or an increase in the concentration of nitric oxide (NO), or cyclic guanosine 3′,5′-monophosphate (cGMP) or both, or an upregulation of the NO-sGC-cGMP pathway is desirable. Compounds useful in the methods of the invention are those of Formula (I) or pharmaceutically acceptable salts thereof.

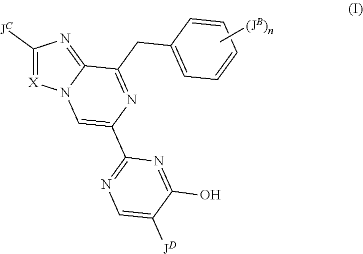

### RELATED APPLICATIONS

This application claims the benefit of priority to U.S. Provisional Application No. 63/177,023, filed on Apr. 20, 2021, and U.S. Provisional Application No. 63/229,251, filed on Aug. 4, 2021. The entire contents of each of the above-referenced applications are incorporated herein by reference.

### FIELD OF THE INVENTION

The present disclosure relates to the use of stimulators of soluble guanylate cyclase (sGC), pharmaceutically acceptable salts thereof and pharmaceutical formulations or dosage forms comprising them, alone or in combination with one or more additional agents, for the treatment of various CNS diseases, wherein an increase in sGC stimulation, or an increase in the concentration of nitric oxide (NO) or cyclic guanosine 3′,5′-monophosphate (cGMP) or both, or an upregulation of the NO-sGC-cGMP pathway is desirable.

### BACKGROUND OF THE INVENTION

sGC is the primary receptor for NO in vivo. Upon binding to sGC, NO activates its catalytic domain and results in the conversion of guanosine-5′-triphosphate (GTP) into the secondary messenger cGMP. The increased level of cGMP, in turn, modulates the activity of downstream effectors including protein kinases, phosphodiesterases (PDEs) and ion channels. In the body, NO is synthesized from arginine and oxygen by various nitric oxide synthase (NOS) enzymes and by sequential reduction of inorganic nitrate. Experimental and clinical evidence indicates that reduced NO concentrations, reduced NO bioavailability and/or reduced responsiveness to endogenously produced NO contributes to the development of numerous diseases. sGC stimulators are heme-dependent agonists of the sGC enzyme that work synergistically with varying amounts of NO to increase its enzymatic conversion of GTP to cGMP. sGC stimulators are clearly differentiated from and structurally unrelated to another class of NO-independent, heme-independent agonists of sGC known as sGC activators.

Therapies that improve or restore the function of sGC offer considerable advantages over current alternative therapies that either target the NO-sGC-cGMP pathway or otherwise benefit from the upregulation of the NO-sGC-cGMP pathway. There is an urgent need to develop new and safe therapies for patients with dysfunctional or downregulated NO-sGC-cGMP pathway.

sGC stimulators that can cross the blood-brain barrier (BBB) and can penetrate into the central nervous system (CNS) provide additional benefits for the treatment of diseases of the CNS. sGC stimulators herein described are useful for the treatment of diseases of the CNS due to their ability to cross the BBB and activate the target in the brain. There is an urgent need to develop additional new therapeutics for the treatment of diseases of the CNS. Use of sGC stimulation in the CNS represents a new mechanism of action to treat these diseases that has just recently started to be explored, due to the past lack of sGC stimulators with CNS penetrant properties.

### SUMMARY OF THE INVENTION

The present invention is based on the discovery that the compounds disclosed herein are sGC stimulators able to penetrate the blood brain barrier (BBB), and are therefore useful for the treatment of CNS diseases. Compounds with related structural features, particularly, with 4-OH substituent on the pyrimidine ring, were previously known only as synthetic intermediates that could be used in the preparation for sGC stimulators having 4-amino substituents on the pyrimidine ring. No medical use had been attributed to this class of compounds in prior disclosures. It was unexpectedly found that the compounds of the present disclosure have potent sGC stimulatory activities and that they are able to penetrate the BBB, increase cGMP concentrations in the brain and show CNS activity in in-vitro and in vivo assays.

In a first aspect, the present invention is directed to a method of treating a CNS disease, CNS health condition or CNS disorder in a subject in need thereof, comprising administering, alone or in combination therapy, a therapeutically effective amount of a compound represented by Formula I:

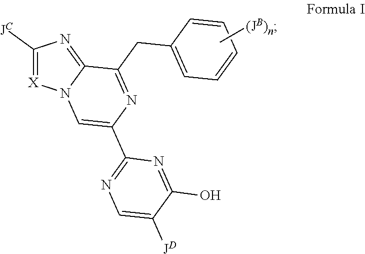

or a pharmaceutically acceptable salt thereof, wherein:
J^{C} is selected from the group consisting of hydrogen, halogen, C₁₋₆ alkyl and C₁₋₆ fluoroalkyl substituted with 1 to 3 fluoro atoms;
X is N or C(J^{C1});
J^{C1} is selected from the group consisting of hydrogen, halogen, C₁₋₆ alkyl and C₁₋₆ fluoroalkyl substituted with 1 to 3 fluoro atoms;
each J^{B} is independently selected from the group consisting of hydrogen, halogenC₁₋₆ alkyl and C₁₋₆ fluoroalkyl substituted with 1 to 3 fluoro atoms;
J^{D} is selected from the group consisting of hydrogen, halogen, C₁₋₆ alkyl and C₁₋₆ fluoroalkyl substituted with 1 to 3 fluoro atoms; and
n is an integer selected from 0, 1, 2, 3 or 4. In a second aspect, the present invention is directed to a method of treating a CNS disease, CNS health condition or CNS disorder in a subject in need thereof, comprising administering, alone or in combination therapy, a therapeutically effective amount of a pharmaceutical composition or a dosage form comprising a compound of Formula I or a pharmaceutically acceptable salt thereof to the subject.

In a third aspect, the invention is further directed to the use of a compound of Formula I or a pharmaceutically acceptable salt thereof, or a pharmaceutical composition or a dosage form comprising the compound of Formula I or a pharmaceutically acceptable salt thereof, for the manufacture of a medicament for the treatment of a CNS disease, CNS health condition CNS or disorder in a subject in need thereof.

In a fourth aspect, the invention is further directed to a compound of Formula I, or a pharmaceutically acceptable salt thereof, a pharmaceutical composition or a dosage form comprising the compound of Formula I or a pharmaceutically acceptable salt thereof for use in the treatment of a CNS disease, CNS health condition or CNS disorder in a subject in need thereof.

### BRIEF DESCRIPTION OF THE DRAWINGS

FIG. 1 shows the effect of compound I-14 on change from baseline in MAP (Δ_{B}MAP) in male, normotensive rats.

FIG. 2 shows the effect of compound I-20 on Δ_{B}MAP in male, normotensive rats.

FIG. 3 shows levels of compound I-14 in the STR and HIPP areas of the brain of adult male Sprague-Dawley rats, following PO (3 mg/kg) administration of compound I-14 at T=0 min. Data are represented as mean±SEM, N=5.

FIG. 4 shows concentration of cGMP in rat's CSF, 1, 2 and 6 hours after administration of a single oral dose of compound I-20 (1 mg/kg, 3 mg/kg or 10 mg/kg).

FIG. 5 shows concentration of cGMP in rat's CSF, 1, 2 and 6 hours after administration of a single oral dose of compound I-14 (1 mg/kg, 3 mg/kg or 10 mg/kg or mg/kg).

FIG. 6 shows cognitive effects of Compound I-14 in the chronic low dose MPTP-lesioned macaque model of cognitive deficits in Parkinson's disease. SDR performance was significantly impaired (**P<0.01) after chronic low dose MPTP exposure. SD (simple discrimination) and SDR (simple discrimination reversal) performance following administration of Vehicle were no different than MPTP Baseline performance. SDR performance was significantly improved (**P<0.01) after Compound I-14 administration and worsened during washout (**P<0.01 vs drug performance). FIG. 6A shows mean±SEM performance; and FIG. 6B is scatter plot showing individual data with mean±SEM. N=Normal, Pre-MPTP; WO=Washout.

### DETAILED DESCRIPTION OF THE INVENTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### Definitions and General Terminology

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

When one or more position(s) of a structure can be substituted with one or more than one substituent selected from a specified group or list, the substituent or substituents at each position may be “independently selected” to be equal or the same at each position and for each instance, unless otherwise specified. For example, if a phenyl is substituted with two instances of R¹⁰⁰, and each R¹⁰⁰ is independently selected from halogen and methyl, that means that each instance of R¹⁰⁰ is separately selected from halogen or methyl; for instance, one R¹⁰⁰ may be fluoro and one may be methyl, or both may be chloro, etc. Similarly, if a substitutable atom is bonded to more than one hydrogen (e.g., CH₃ or NH₂), the substitutents may be “independently selected” to be equal or the same at each position and for each instance, unless otherwise specified. For example, if a methyl (e.g., CH₃) is substituted with two instances of R¹⁰⁰, and each R¹⁰⁰ is independently selected from halogen and methyl, that means that each instance of R¹⁰⁰ is separately selected from halogen or methyl; for instance, one R¹⁰⁰ may be fluoro and one may be methyl (e.g., CHF(CH₃), or both may be chloro (e.g., CHCl₂), etc.

Selection of substituents and combinations envisioned by this disclosure are only those that result in the formation of stable or chemically feasible compounds. Such choices and combinations will be apparent to those of ordinary skill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. A chemically feasible compound is a compound that can be prepared by a person skilled in the art based on the disclosures herein, supplemented, if necessary, by relevant knowledge of the art.

Unless otherwise stated, all tautomeric forms of the compounds of the present disclosure are also within the scope of the invention.

In one embodiment, the present disclosure may include replacement of hydrogen with deuterium (i.e., ²H), which may afford certain therapeutic advantages resulting from greater metabolic stability (e.g., increased in vivo half-life or reduced dosage requirements) and hence may be preferred in some circumstances. Deuterium labeled compounds of the present invention can generally be prepared by following procedures analogous to those disclosed in the Schemes and/or in the Examples herein below, by substituting a deuterated reagent for a non-deuterated reagent.

The term “alkyl” as in, for example, “alkyl chain” or “alkyl group”, as used herein, refers to a saturated unbranched (e.g., linear) or branched monovalent hydrocarbon radical. A Cₓ alkyl is an alkyl chain containing x carbon atoms, wherein x is an integer different from 0. A “C_{x-y} alkyl”, wherein x and y are two different integers, both different from 0, is an alkyl chain containing between x and y number of carbon atoms, inclusive. For example, a C₁₋₆ alkyl is an alkyl as defined above containing any number of between 1 and 6 carbon atoms. Examples of alkyl groups include, but are not limited to, methyl (i.e., C₁ alkyl), ethyl (i.e., C₂ alkyl), n-propyl (a C₃ alkyl), isopropyl (a different C₃ alkyl), n-butyl, isobutyl, s-butyl, t-butyl, pentyl, hexyl, heptyl, octyl and the like. In certain embodiments, the alkyl group is a C₁₋₄ alkyl. In certain embodiments, the alkyl group is a C₁₋₃ alkyl or C₁₋₂ alkyl. In still other embodiments, the alkyl group is methyl or ethyl

The term “fluoroalkyl”, as used herein, refers to an alkyl group as defined above in which one or more of the hydrogen atoms attached to the chain carbon atoms has been replaced by fluoro at any one or more carbon atoms of the alkyl group. For example, a fluoroalkyl substituted with 1 to 3 fluoro atoms is an alkyl group in which 1 to 3 hydrogen atoms have been replaced with fluoro atoms at any position, either on the same carbon atom or different carbon atoms of the alkyl chain.

As used herein, the terms “halogen” or “halo” mean F, Cl, Br, or I. In certain embodiments, the halo is F or Cl. In still other embodiments, the halo is F.

The term “hydroxyl” or “hydroxy” refers to —OH.

The compounds of the invention are defined herein by their chemical structures and/or chemical names. Where a compound is referred to by both a chemical structure and a chemical name, and the chemical structure and chemical name conflict, the chemical structure is determinative of the compound's identity.

### Compounds and Composition Embodiments

The present invention is directed to medical uses of compounds of Formula I, pharmaceutically acceptable salts thereof, or pharmaceutical compositions comprising the compounds or their pharmaceutically acceptable salts.

In a first aspect, the present invention is directed to a method of treating a CNS disease, health condition or disorder in a subject in need thereof, comprising administering to the subject, alone or in combination therapy, a therapeutically effective amount of a compound represented by Formula I:

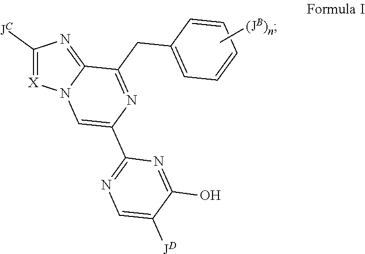

or a pharmaceutically acceptable salt thereof, wherein:
J^{C} is selected from the group consisting of hydrogen, halogenC₁₋₆ alkyl and C₁₋₆ fluoroalkyl substituted with 1 to 3 fluoro atoms;
X is N or C(J^{C1});
J^{C1} is selected from the group consisting of hydrogen, halogen, C₁₋₆ alkyl and C₁₋₆ fluoroalkyl substituted with 1 to 3 fluoro atoms;
each J^{B} is independently selected from the group consisting of hydrogen, halogen, C₁₋₆ alkyl and C₁₋₆ fluoroalkyl substituted with 1 to 3 fluoro atoms;
J^{D} is selected from the group consisting of hydrogen, halogen, C₁₋₆ alkyl and C₁₋₆ fluoroalkyl substituted with 1 to 3 fluoro atoms; and
n is an integer selected from 0, 1, 2, 3 or 4.

In a second aspect, the present invention is directed to a method of treating a CNS disease, health condition or disorder in a subject in need thereof, comprising administering, alone or in combination therapy, a therapeutically effective amount of a pharmaceutical composition or dosage form comprising a compound of Formula I or a pharmaceutically acceptable salt thereof to the subject.

In a third aspect, the invention is further directed to the use of a compound of Formula I or a pharmaceutically acceptable salt thereof, or a pharmaceutical composition or a dosage form comprising the compound of Formula I or a pharmaceutically acceptable salt thereof, for the manufacture of a medicament for the treatment of a CNS disease, health condition or disorder in a subject in need thereof.

In a fourth aspect, the invention is further directed to a compound of Formula I, or a pharmaceutically acceptable salt thereof, or a pharmaceutical composition or a dosage form comprising the compound of Formula I or a pharmaceutically acceptable salt thereof for use in the treatment of a CNS disease, health condition or disorder in a subject in need thereof.

In a first embodiment of the first, second, third and fourth aspects, for a compound of Formula I, n is an integer selected from 1, 2, 3 or 4, each J^{B} is independently selected from the group consisting of halogen, C₁₋₆ alkyl and C₁₋₆ fluoroalkyl substituted with 1 to 3 fluoro atoms, all other carbon atoms of the phenyl ring are unsubstituted, and the remaining variables are as defined above.

In a second embodiment of the first, second, third and fourth aspects, for a compound of Formula I, or a pharmaceutically acceptable salt thereof, J^{C} is selected from the group consisting of hydrogen, halogen and C₁₋₆ alkyl; J^{C1} is selected from the group consisting of hydrogen, halogen and C₁₋₆ alkyl; each J^{B} is independently selected from the group consisting of hydrogen, halogen and C₁₋₆ alkyl; J^{D} is selected from the group consisting of hydrogen, halogen and C₁₋₆ alkyl; and the remaining variables are as defined above for Formula I in the first aspect.

In a third embodiment of the first, second, third and fourth aspects, the compound of Formula I is represented by Formula IA:

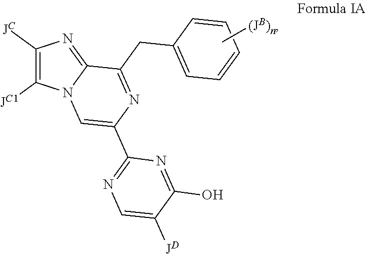

or a pharmaceutically acceptable salt thereof, wherein the variables are as described above for Formula I in the first aspect or in the first or second embodiment.

In a fourth embodiment, for the compound of Formula IA, or a pharmaceutically acceptable salt thereof, J^{C1} is H, F or Cl; and the remaining variables are as defined in the first aspect or in the first, second or third embodiment.

In a fifth embodiment, for the compound of Formula IA, or a pharmaceutically acceptable salt thereof, J^{C1} is H; and the remaining variables are defined in the first aspect or in any one of the first to fourth embodiments.

In a sixth embodiment, for the compound of Formula IA, or a pharmaceutically acceptable salt thereof, J^{C1} is F; and the remaining variables are defined in the first aspect or in any one of the first to fifth embodiments.

In an seventh embodiment, the compound of Formula I is represented by Formula IB:

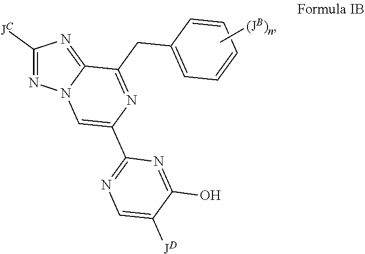

or a pharmaceutically acceptable salt thereof, wherein the variables are as described above for Formula I, according to the first aspect or any one of the first to sixth embodiments.

In an eighth embodiment, for the compound of Formula I, IA or IB, or a pharmaceutically acceptable salt thereof, n is 2 or 3, and the remaining variables are as described in the first aspect or the first, second, third, fourth, fifth, sixth or seventh embodiment.

In a ninth embodiment, for the compound of Formula I, IA or IB, or a pharmaceutically acceptable salt thereof, n is 0 or 1, and the remaining variables are as described in the first aspect or the first, second, third, fourth, fifth, sixth or seventh embodiment. In some embodiments, each J^{B} is independently halogen or C₁₋₆ alkyl.

In a tenth embodiment, for the compound of Formula I, IA or IB, or a pharmaceutically acceptable salt thereof, each J^{B} is independently H, F or C₁₋₄ alkyl; and the remaining variables are as described in the first aspect or the first, second, third, fourth, fifth, sixth, seventh, eighth or ninth embodiment. In some embodiments, each J^{B} is independently F or C₁₋₄ alkyl.

In an eleventh embodiment, for the compound of Formula I, IA or IB, or a pharmaceutically acceptable salt thereof, n is 2 or 3; each J^{B} is independently F or methyl; and the remaining variables are as described in the first aspect or the first, second, third, fourth, or fifth, sixth or seventh embodiment.

In a twelfth embodiment, for the compound of Formula I, IA or IB, or a pharmaceutically acceptable salt thereof, n is 2; J^{B} are both F or one of J^{B} is F and the other is methyl; and the remaining variables are as described in the first aspect or the first, second, third, fourth, fifth, sixth or seventh embodiment. In some embodiments, one J^{B} is F and the other is methyl.

In a thirteenth embodiment, for the compound of Formula I, IA or IB, or a pharmaceutically acceptable salt thereof, n is 3; two of J^{B} are F and the other is methyl; and the remaining variables are as described in the first aspect or the first, second, third, fourth, fifth, sixth or seventh embodiment. In some embodiments, J^{D} is H or F. In some embodiments, J^{D} is F. In some embodiments, J^{D} is H.

In a fourteenth embodiment, for the compound of Formula I, IA or IB, or a pharmaceutically acceptable salt thereof, n is 1; J^{B} is F; and the remaining variables are as described in the first aspect or the first, second, third, fourth, fifth, sixth or seventh embodiment.

In a fifteenth embodiment, for the compound of Formula I, IA or IB, or a pharmaceutically acceptable salt thereof, n is 0; and the remaining variables are as described in the first aspect or the first, second, third, fourth, fifth, sixth or seventh embodiment.

In a sixteenth embodiment, for the compound of Formula I, IA or IB, or a pharmaceutically acceptable salt thereof, J^{D} is hydrogen; and the remaining variables are as described in the first aspect or the first, second, third, fourth, fifth, sixth, seventh, eighth, ninth, tenth, eleventh, twelfth, thirteenth, fourteenth or fifteenth embodiment.

In a seventeenth embodiment, for the compound of Formula I, IA or IB, or a pharmaceutically acceptable salt thereof, J^{D} is F, Cl or methyl; and the remaining variables are as described in the first aspect or the first, second, third, fourth, fifth, sixth, seventh, eighth, ninth, tenth, eleventh, twelfth, thirteenth, fourteenth or fifteenth embodiment.

In an eighteenth embodiment, for the compound of Formula I, IA or IB, or a pharmaceutically acceptable salt thereof, J^{D} is F; and the remaining variables are as described in the first aspect or the first, second, third, fourth, fifth, sixth, seventh, eighth, ninth, tenth, eleventh, twelfth, thirteenth, fourteenth or fifteenth embodiment.

In a nineteenth embodiment, for the compound of Formula I, IA or IB, or a pharmaceutically acceptable salt thereof, J^{C} is H, Cl or F; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth or eighteenth embodiment.

In a twentieth embodiment, for the compound of Formula I, IA or IB, or a pharmaceutically acceptable salt thereof, J^{C} is H; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth or eighteenth embodiment.

In a twenty-first embodiment, the compound of Formula I is a compound shown in Table I, or a pharmaceutically acceptable salt thereof.

TABLE I
Exemplary sGC stimulators.

| | |
|---|---|
| 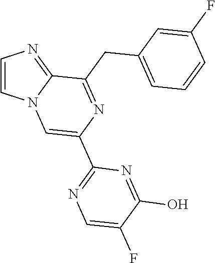 [SMILES: Oc1nc(-c2cn3ccnc3c(Cc3cccc(F)c3)n2)ncc1F] | I-1 |
| 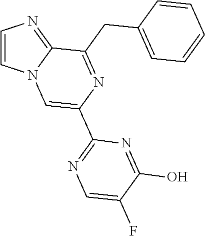 [SMILES: Oc1nc(-c2cn3ccnc3c(Cc3ccccc3)n2)ncc1F] | I-2 |
| 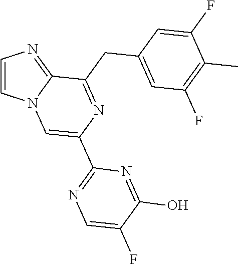 [SMILES: Cc1c(F)cc(Cc2nc(-c3ncc(F)c(O)n3)cn3ccnc23)cc1F] | I-3 |
| 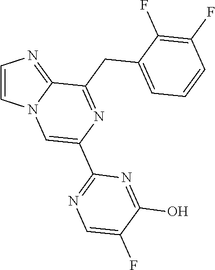 [SMILES: Oc1nc(-c2cn3ccnc3c(Cc3cccc(F)c3F)n2)ncc1F] | I-4 |
| 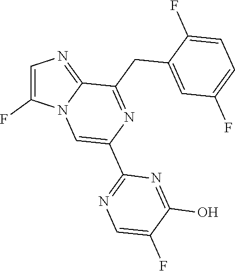 [SMILES: Oc1nc(-c2cn3c(F)cnc3c(Cc3cc(F)ccc3F)n2)ncc1F] | I-5 |
| 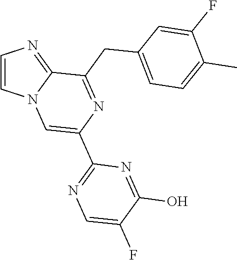 [SMILES: Cc1ccc(Cc2nc(-c3ncc(F)c(O)n3)cn3ccnc23)cc1F] | I-6 |
| 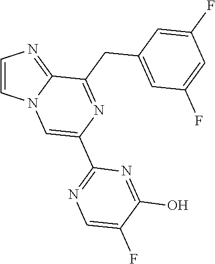 [SMILES: Oc1nc(-c2cn3ccnc3c(Cc3cc(F)cc(F)c3)n2)ncc1F] | I-7 |
| 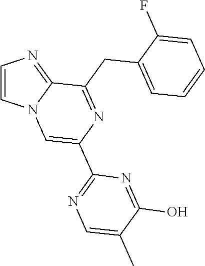 [SMILES: Cc1cnc(-c2cn3ccnc3c(Cc3ccccc3F)n2)nc1O] | I-8 |
| 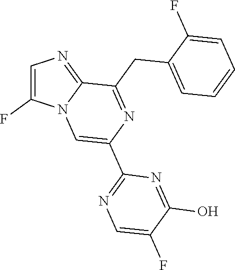 [SMILES: Oc1nc(-c2cn3c(F)cnc3c(Cc3ccccc3F)n2)ncc1F] | I-9 |
| 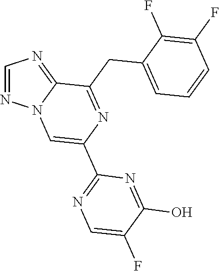 [SMILES: Oc1nc(-c2cn3ncnc3c(Cc3cccc(F)c3F)n2)ncc1F] | I-10 |
| 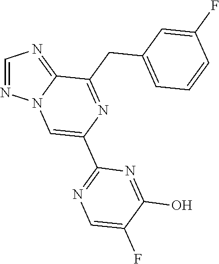 [SMILES: Oc1nc(-c2cn3ncnc3c(Cc3cccc(F)c3)n2)ncc1F] | I-11 |
| 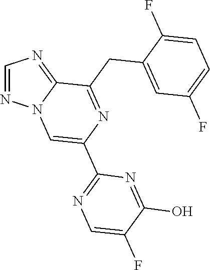 [SMILES: Oc1nc(-c2cn3ncnc3c(Cc3cc(F)ccc3F)n2)ncc1F] | I-12 |
| 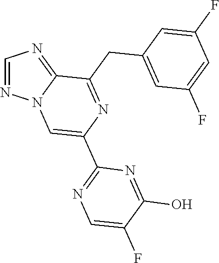 [SMILES: Oc1nc(-c2cn3ncnc3c(Cc3cc(F)cc(F)c3)n2)ncc1F] | I-13 |
| 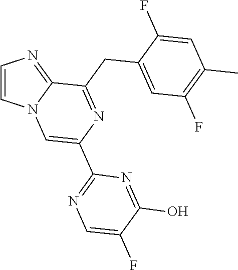 [SMILES: Cc1cc(F)c(Cc2nc(-c3ncc(F)c(O)n3)cn3ccnc23)cc1F] | I-14 |
| 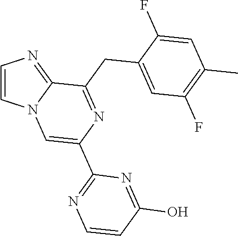 [SMILES: Cc1cc(F)c(Cc2nc(-c3nccc(O)n3)cn3ccnc23)cc1F] | I-15 |
| 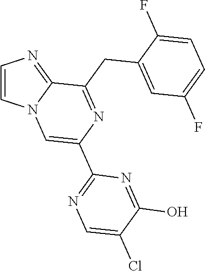 [SMILES: Oc1nc(-c2cn3ccnc3c(Cc3cc(F)ccc3F)n2)ncc1Cl] | I-16 |
| 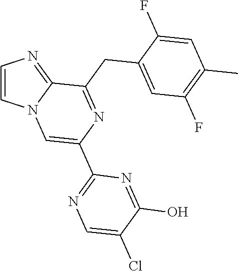 [SMILES: Cc1cc(F)c(Cc2nc(-c3ncc(Cl)c(O)n3)cn3ccnc23)cc1F] | I-17 |
| 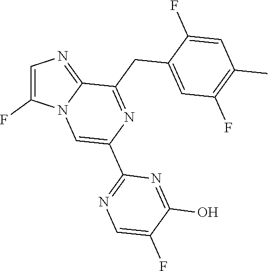 [SMILES: Cc1cc(F)c(Cc2nc(-c3ncc(F)c(O)n3)cn3c(F)cnc23)cc1F] | I-18 |
| 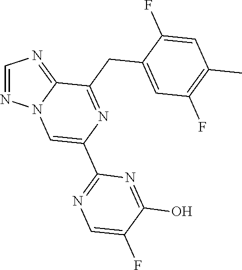 [SMILES: Cc1cc(F)c(Cc2nc(-c3ncc(F)c(O)n3)cn3ncnc23)cc1F] | I-19 |
| 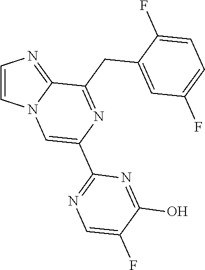 [SMILES: Oc1nc(-c2cn3ccnc3c(Cc3cc(F)ccc3F)n2)ncc1F] | I-20 |
| 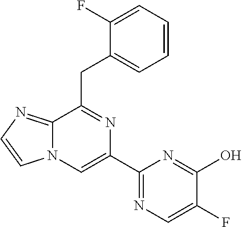 [SMILES: Oc1nc(-c2cn3ccnc3c(Cc3ccccc3F)n2)ncc1F] | I-21 |
| 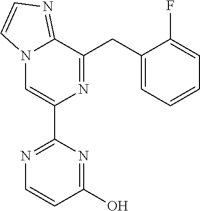 [SMILES: Oc1ccnc(-c2cn3ccnc3c(Cc3ccccc3F)n2)n1] | I-22 |
| 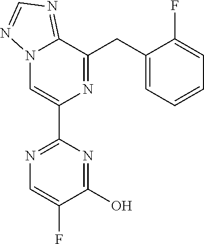 [SMILES: Oc1nc(-c2cn3ncnc3c(Cc3ccccc3F)n2)ncc1F] | I-23 |
| 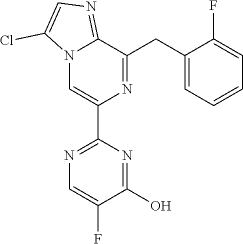 [SMILES: Oc1nc(-c2cn3c(Cl)cnc3c(Cc3ccccc3F)n2)ncc1F] | I-24 |
| 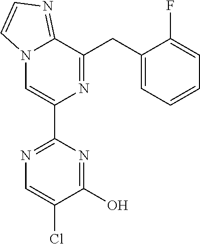 [SMILES: Oc1nc(-c2cn3ccnc3c(Cc3ccccc3F)n2)ncc1Cl] | I-25 |
| 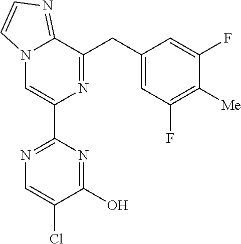 [SMILES: Cc1c(F)cc(Cc2nc(-c3ncc(Cl)c(O)n3)cn3ccnc23)cc1F] | I-26 |

or a pharmaceutically acceptable salt thereof.

In a twenty-second embodiment, for the methods and uses of the present invention, the sGC stimulator is compound 1-14, or a pharmaceutically acceptable salt thereof. In one embodiment, the pharmaceutically acceptable salt is a sodium salt. In another embodiment, the sGC stimulator is sodium salt of compound 1-14 represented by the following formula:

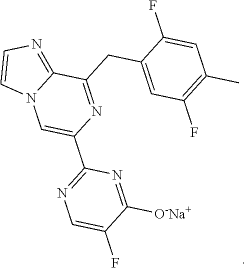

In a twenty-third embodiment, for the methods and uses of the present invention, the sGC stimulator is compound 1-20, or a pharmaceutically acceptable salt thereof. In one embodiment, the pharmaceutically acceptable salt is a sodium salt. In another embodiment, the sGC stimulator is sodium salt of compound I-20 represented by the following formula:

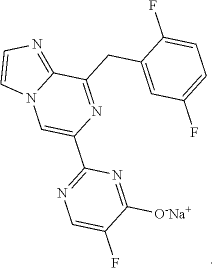

In a twenty-fourth embodiment, the compound of the present invention is a compound represented by Formula IC:

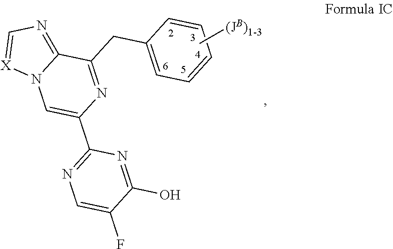

or a pharmaceutically acceptable salt thereof, wherein X is N or C(J^{C1}), wherein when X is C(J^{C1}), it is represented by C in the below table; and the definitions for variables X, J^{C1} and J^{B} are described in the Table below; further where Me represents a methyl group and Me-F represents a fluorinated methyl group substituted by 1 to 3 fluoro atoms (i.e., —CH₂F, —CHF₂ or —CF₃):

| | position of J^{B} on the phenyl | | | | | | |
|---|---|---|---|---|---|---|---|
| Compound | ring and J^{B} defintion | | | | | | |
| No. | 2 | 3 | 4 | 5 | 6 | X | J^{C1} |
| I-27 | | F | | | | C | F |
| I-28 | | | F | | | C | H |
| I-29 | | | F | | | N | — |
| I-30 | | | F | | | C | F |
| I-31 | F | F | | | | C | F |
| I-32 | F | | F | | | C | H |
| I-33 | F | | F | | | N | — |
| I-34 | F | | F | | | C | F |
| I-35 | F | | | | F | C | H |
| I-36 | F | | | | F | N | — |
| I-37 | F | | | | F | C | F |
| I-38 | | F | F | | | C | H |
| I-39 | | F | F | | | N | — |
| I-40 | | F | F | | | C | F |
| I-41 | | F | | F | | C | F |
| I-42 | | F | | | F | C | H |
| I-43 | | F | | | F | N | — |
| I-44 | | F | | | F | C | F |
| I-45 | F | Me | | | | C | H |
| I-46 | F | Me | | | | N | — |
| I-47 | F | Me | | | | C | F |
| I-48 | Me | F | | | | C | H |
| I-49 | Me | F | | | | N | — |
| I-50 | Me | F | | | | C | F |
| I-51 | F | | Me | | | C | H |
| I-52 | F | | Me | | | N | — |
| I-53 | F | | Me | | | C | F |
| I-54 | Me | | F | | | C | H |
| I-55 | Me | | F | | | N | — |
| I-56 | Me | | F | | | C | F |
| I-57 | F | | | Me | | C | H |
| I-58 | F | | | Me | | C | F |
| I-59 | F | | | Me | | N | — |
| I-60 | Me | | | F | | C | H |
| I-61 | Me | | | F | | C | F |
| I-62 | Me | | | F | | N | — |
| I-63 | F | | | | Me | C | H |
| I-64 | F | | | | Me | N | — |
| I-65 | F | | | | Me | C | F |
| I-66 | Me | | | | F | C | H |
| I-67 | Me | | | | F | N | — |
| I-68 | Me | | | | F | C | F |
| I-69 | | F | Me | | | N | — |
| I-70 | | F | Me | | | C | F |
| I-71 | | Me | F | | | C | H |
| I-72 | | Me | F | | | N | — |
| I-73 | | Me | F | | | C | F |
| I-74 | | F | | Me | | C | H |
| I-75 | | F | | Me | | N | — |
| I-76 | | F | | Me | | C | F |
| I-77 | | Me | | F | | C | H |
| I-78 | | Me | | F | | N | — |
| I-79 | | Me | | F | | C | F |
| I-80 | | Me | | | F | C | H |
| I-81 | | Me | | | F | N | — |
| I-82 | | Me | | | F | C | F |
| I-83 | | F | | | Me | C | H |
| I-84 | | F | | | Me | N | — |
| I-85 | | F | | | Me | C | F |
| I-86 | F | F | Me | | | C | H |
| I-87 | F | F | Me | | | N | — |
| I-88 | F | F | Me | | | C | F |
| I-89 | F | F | | Me | | C | H |
| I-90 | F | F | | Me | | N | — |
| I-91 | F | F | | Me | | C | F |
| I-92 | F | F | | | Me | C | H |
| I-93 | F | F | | | Me | N | — |
| I-94 | F | F | | | Me | C | F |
| I-95 | F | Me | F | | | C | H |
| I-96 | F | Me | F | | | N | — |
| I-97 | F | Me | F | | | C | F |
| I-98 | F | | F | Me | | C | H |
| I-99 | F | | F | Me | | N | — |
| I-100 | F | | F | Me | | C | F |
| I-101 | F | | F | | Me | C | H |
| I-102 | F | | F | | Me | N | — |
| I-103 | F | | F | | Me | C | F |
| I-104 | F | Me | | F | | C | H |
| I-105 | F | Me | | F | | C | F |
| I-106 | F | Me | | F | | N | — |
| I-107 | F | | | F | Me | C | H |
| I-108 | F | | | F | Me | C | F |
| I-109 | F | | | F | Me | N | — |
| I-110 | F | Me | | | F | C | H |
| I-111 | F | Me | | | F | N | — |
| I-112 | F | Me | | | F | C | F |
| I-113 | F | | Me | | F | C | H |
| I-114 | F | | Me | | F | N | — |
| I-115 | F | | Me | | F | C | F |
| I-116 | Me | F | F | | | C | H |
| I-117 | Me | F | F | | | N | — |
| I-118 | Me | F | F | | | C | F |
| I-119 | | F | F | Me | | C | H |
| I-120 | | F | F | Me | | N | — |
| I-121 | | F | F | Me | | C | F |
| I-122 | | F | F | | Me | C | H |
| I-123 | | F | F | | Me | N | — |
| I-124 | | F | F | | Me | C | F |
| I-125 | Me | F | | F | | C | H |
| I-126 | Me | F | | F | | N | — |
| I-127 | Me | F | | F | | C | F |
| I-128 | | F | Me | F | | N | — |
| I-129 | | F | Me | F | | C | F |
| I-130 | F | Me—F | | | | C | H |
| I-131 | F | Me—F | | | | N | — |
| I-132 | F | Me—F | | | | C | F |
| I-133 | Me—F | F | | | | C | H |
| I-134 | Me—F | F | | | | N | — |
| I-135 | Me—F | F | | | | C | F |
| I-136 | F | | Me—F | | | C | H |
| I-137 | F | | Me—F | | | N | — |
| I-138 | F | | Me—F | | | C | F |
| I-139 | Me—F | | F | | | C | H |
| I-140 | Me—F | | F | | | N | — |
| I-141 | Me—F | | F | | | C | F |
| I-142 | F | | | Me-F | | C | H |
| I-143 | F | | | Me-F | | C | F |
| I-144 | F | | | Me-F | | N | — |
| I-145 | Me—F | | | F | | C | H |
| I-146 | Me—F | | | F | | C | F |
| I-147 | Me—F | | | F | | N | — |
| I-148 | F | | | | Me-F | C | H |
| I-149 | F | | | | Me-F | N | — |
| I-150 | F | | | | Me-F | C | F |
| I-151 | Me—F | | | | F | C | H |
| I-152 | Me—F | | | | F | N | — |
| I-153 | Me—F | | | | F | C | F |
| I-154 | | F | Me—F | | | N | — |
| I-155 | | F | Me—F | | | C | F |
| I-156 | | F | Me—F | | | C | H |
| I-157 | | Me—F | F | | | N | — |
| I-158 | | Me—F | F | | | C | H |
| I-159 | | Me—F | F | | | C | F |
| I-160 | | F | | Me-F | | C | H |
| I-161 | | F | | Me-F | | N | — |
| I-162 | | F | | Me-F | | C | F |
| I-163 | | Me—F | | F | | C | H |
| I-164 | | Me—F | | F | | N | — |
| I-165 | | Me—F | | F | | C | F |
| I-166 | | Me—F | | | F | C | H |
| I-167 | | Me—F | | | F | N | — |
| I-168 | | Me—F | | | F | C | F |
| I-169 | | F | | | Me-F | C | H |
| I-170 | | F | | | Me-F | N | — |
| I-171 | | F | | | Me-F | C | F |
| I-172 | F | F | Me—F | | | C | H |
| I-173 | F | F | Me—F | | | N | — |
| I-174 | F | F | Me—F | | | C | F |
| I-175 | F | F | | Me-F | | C | H |
| I-176 | F | F | | Me-F | | N | — |
| I-177 | F | F | | Me-F | | C | F |
| I-178 | F | F | | | Me-F | C | H |
| I-179 | F | F | | | Me-F | N | — |
| I-180 | F | F | | | Me-F | C | F |
| I-181 | F | Me—F | F | | | C | H |
| I-182 | F | Me—F | F | | | N | — |
| I-183 | F | Me—F | F | | | C | F |
| I-184 | F | | F | Me-F | | C | H |
| I-185 | F | | F | Me-F | | N | — |
| I-186 | F | | F | Me-F | | C | F |
| I-187 | F | | F | | Me-F | C | H |
| I-188 | F | | F | | Me-F | N | — |
| I-189 | F | | F | | Me-F | C | F |
| I-190 | F | Me—F | | F | | C | H |
| I-191 | F | Me—F | | F | | C | F |
| I-192 | F | Me—F | | F | | N | — |
| I-193 | F | | | F | Me-F | C | H |
| I-194 | F | | | F | Me-F | C | F |
| I-195 | F | | | F | Me-F | N | — |
| I-196 | F | Me—F | | | F | C | H |
| I-197 | F | Me—F | | | F | N | — |
| I-198 | F | Me—F | | | F | C | F |
| I-199 | F | | Me—F | | F | C | H |
| I-200 | F | | Me—F | | F | N | — |
| I-201 | F | | Me—F | | F | C | F |
| I-202 | Me—F | F | F | | | C | H |
| I-203 | Me—F | F | F | | | N | — |
| I-204 | Me—F | F | F | | | C | F |
| I-205 | | F | F | Me-F | | C | H |
| I-206 | | F | F | Me-F | | N | — |
| I-207 | | F | F | Me-F | | C | F |
| I-208 | | F | F | | Me-F | C | H |
| I-209 | | F | F | | Me-F | N | — |
| I-210 | | F | F | | Me-F | C | F |
| I-211 | Me—F | F | | F | | C | H |
| I-212 | Me—F | F | | F | | N | — |
| I-213 | Me—F | F | | F | | C | F |
| I-214 | | F | Me—F | F | | N | — |
| I-215 | | F | Me—F | F | | C | F |
| I-216 | | F | Me—F | F | | C | H |

### Pharmaceutically Acceptable Salts of the Invention.

A “pharmaceutically acceptable salt” of the compounds described herein include those derived from said compounds when mixed with inorganic or organic acids or bases. In some embodiments, the salts can be prepared in situ during the final isolation and purification of the compounds. In other embodiments the salts can be prepared from the free form of the compound in a separate synthetic step. The preparation of the pharmaceutically acceptable salts described above and other typical pharmaceutically acceptable salts is more fully described by Berg et al., “Pharmaceutical Salts,” J. Pharm. Sci., 1977:66:1-19, incorporated here by reference in its entirety. The pharmaceutically acceptable salts of a compound of Formula I are those that may be used in medicine. Salts that are not pharmaceutically acceptable may, however, be useful in the preparation of a compound of Formula I or of their pharmaceutically acceptable salts.

When a compound of Formula I is acidic, suitable “pharmaceutically acceptable salts” refers to salts prepared from pharmaceutically acceptable non-toxic bases including inorganic and organic bases. Salts derived from inorganic bases include aluminum, ammonium, calcium, copper, ferric, ferrous, lithium, magnesium, manganic, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines, arginine, betaine, caffeine, choline, N, Ni-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

In some embodiments, compounds of Formula I have an acidic OH group that can react with a base (e.g., a pharmaceutically acceptable non-toxic base) to form a salt (e.g., a pharmaceutically acceptable salt). In some embodiments, the salt is an ammonium, calcium, magnesium, potassium or sodium salt. In other embodiments, the salt is a sodium salt.

When a compound of Formula I is basic, salts may be prepared from pharmaceutically acceptable non-toxic acids, including inorganic and organic acids. Such acids include acetate, acetic, acid citrate, acid phosphate, ascorbate, benzenesulfonic, benzenesulfonate, benzoic, benzoate, bromide, bisulfate, bitartrate, camphorsulfonic, chloride, citrate, citric, ethanesulfonate, ethanesulfonic, formate, fumarate, fumaric, gentisinate, gluconate, gluconic, glucuronate, glutamate, glutamic, hydrobromic, hydrochloric, iodide, isethionic, isonicotinate, lactate, lactic, maleate, maleic, malic, mandelic, methanesulfonic, methanesulfonate, mucic, nitrate, nitric, oleate, oxalate, pamoic, pamoate (i.e., 1,1′-methylene-bis-(2-hydroxy-3-naphthoate)), pantothenic, pantothenate, phosphate, phosphoric, saccharate, salicylate, succinic, succinate, sulfuric, sulfate, tannate, tartrate, tartaric, p-toluenesulfonate, p-toluenesulfonic acid and the like. Particular embodiments include citric, hydrobromic, hydrochloric, maleic, phosphoric, sulfuric and tartaric acids.

In addition to the compounds described herein, their pharmaceutically acceptable salts may also be employed in compositions or dosage forms to treat or prevent the herein identified diseases.

### Pharmaceutical Compositions, Dosage Forms and Methods of Administration.

The compounds herein disclosed, and their pharmaceutically acceptable salts thereof may be formulated as pharmaceutical compositions or “formulations” for the treatments and uses of the invention.

A typical formulation is prepared by mixing a compound of Formula I, or a pharmaceutically acceptable salt thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which a compound of Formula I is being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., a compound of Formula I or pharmaceutical composition thereof) or aid in the manufacturing of the pharmaceutical product (i.e., medicament).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively; in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

The formulations may be prepared using conventional dissolution and mixing procedures. The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease, or one or more of its symptoms.

The terms “administer”, “administering” or “administration” in reference to a compound, composition or dosage form of the invention means introducing the compound into the system of the subject or patient in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g. orally (including, but not limited to solid dosage forms including hard or soft capsules (e.g. gelatin capsules), tablets, pills, powders, sublingual tablets, troches, lozenges, and granules; and liquid dosage forms including, but are not limited to, pharmaceutically acceptable emulsions, microemulsions, aqueous or oil solutions, suspensions, syrups and elixirs, by inhalation (e.g. with an aerosol, gas, inhaler, nebulizer or the like), to the ear (e.g. using ear drops), topically (e.g. using creams, gels, inhalants, liniments, lotions, ointments, patches, pastes, powders, solutions, sprays, transdermal patches, etc.), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc.), via ear drops, via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Formulations of a compound intended for oral use may be prepared according to any method known to the art for the manufacture of pharmaceutical compositions.

In solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

In addition to the active compounds, liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Oral compositions (either solid or liquid) can also include excipients and adjuvants such as dispersing or wetting agents, such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate); emulsifying and suspending agents, such as sodium carboxymethylcellulose, croscarmellose, povidone, methylcellulose, hydroxypropyl methylcelluose, sodium alginate, polyvinylpyrrolidone, gum tragacanth and gum acacia; sweetening, flavoring, and perfuming agents; and/or one or more preservatives such as ethyl or n-propyl p-hydroxy-benzoate, one or more coloring agents, one or more flavoring agents and one or more sweetening agents, such as sucrose or saccharin.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a particle size for example in the range of 0.1 to 500 micros (including particles in a range between 0.1 and 500 microns in increments microns such as 0.5, 1, 30, 35 microns, etc.) which is administered by rapid inhalation through the nasal passage or by inhalation through the mouth so as to reach the alveolar sacs.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs. The active component is admixed under sterile conditions with a pharmaceutically acceptable carrier and any needed preservatives or buffers as may be required.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof.

Topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using a compound of Table I may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of a compound of Formula I include Tween™-60, Span™-80, cetostearyl alcohol, benzyl alcohol, myristyl alcohol, glyceryl mono-stearate and sodium lauryl sulfate.

Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

Sterile injectable forms of the compositions described herein (e.g. for parenteral administration) may be aqueous or oleaginous suspension. These suspensions may be formulated according to techniques known in the art using suitable dispersing or wetting agents and suspending agents (including those described in the preceding paragraph). The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as a vegetable oil, for example arachis oil, olive oil, sesame oil or coconut oil, especially in their polyoxyethylated versions, or in mineral oil such as liquid paraffin. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

In another aspect, a compound of Formula I or a pharmaceutically acceptable salt thereof may be formulated in a veterinary composition comprising a veterinary carrier. Veterinary carriers are materials useful for the purpose of administering the composition and may be solid, liquid or gaseous materials which are otherwise inert. In the veterinary art and are compatible with the active ingredient. These veterinary compositions may be administered parenterally, orally or by any other desired route.

### Therapeutic Methods

In a first aspect, the present invention is directed to a method of treating a CNS disease, health condition or disorder in a subject in need thereof, comprising administering, alone or in combination therapy, a therapeutically effective amount of a compound of Formula I or a pharmaceutically acceptable salt thereof to the subject. In certain embodiments, the compound of Formula I is as described in any one of the first to twenty-first embodiments above.

In a second aspect, the present invention is directed to a method of treating a CNS disease, health condition or disorder in a subject in need thereof, comprising administering, alone or in combination therapy, a therapeutically effective amount of a pharmaceutical composition or dosage form comprising a compound of Formula I or a pharmaceutically acceptable salt thereof to the subject. In certain embodiments, the compound of Formula I is as described in any one of the first to twenty-first embodiments above.

In a third aspect, the invention is further directed to the use of a compound of Formula I or a pharmaceutically acceptable salt thereof, or a pharmaceutical composition or a dosage form comprising the compound of Formula I or a pharmaceutically acceptable salt thereof, for the manufacture of a medicament for the treatment of a CNS disease, health condition or disorder in a subject in need thereof. In certain embodiments, the compound of Formula I is as described in any one of the first to twenty-first embodiments above.

In a fourth aspect, the invention is further directed to a compound of Formula I, or a pharmaceutically acceptable salt thereof, or a pharmaceutical composition or a dosage form comprising the compound of Formula I or a pharmaceutically acceptable salt thereof, for use in the treatment of a CNS disease, health condition or disorder in a subject in need thereof. In certain embodiments, the compound of Formula I is as described in any one of the first to twenty-first embodiments above.

In one embodiment of the first, second, third and fourth aspects, the compounds here disclosed are sGC stimulators that may be useful in the prevention and/or treatment of diseases and disorders characterized by increased neuroinflammation. One embodiment of the invention is a method of decreasing neuroinflammation in a subject in need thereof by administering to the subject any one of the compounds of Formula I or a pharmaceutically acceptable salt thereof, or a pharmaceutical composition or a dosage form comprising it.

In another embodiment of the first to fourth aspects of the invention, the compounds here disclosed are sGC stimulators that may be useful in the prevention and/or treatment of diseases and disorders characterized by increased neurotoxicity. One embodiment of the invention is a method of reducing or compensating the negative effects neurotoxicity in a subject in need thereof by administering to the subject any one of the compounds of Formula I or a pharmaceutically acceptable salt thereof, or a pharmaceutical composition or a dosage form comprising it.

In another embodiment of the first to fourth aspects of the invention, the compounds here disclosed are sGC stimulators that may be useful in the prevention and/or treatment of diseases and disorders characterized by impaired neuroregeneration. One embodiment of the invention is a method of restoring neuroregeneration in a subject in need thereof by administering to the subject any one of the compounds of Formula I or a pharmaceutically acceptable salt thereof, or a pharmaceutical composition or a dosage form comprising it.

In another embodiment of the first to fourth aspects of the invention, the compounds here disclosed are sGC stimulators that may be useful in the prevention and/or treatment of diseases and disorders characterized by impaired synaptic function. One embodiment of the invention is a method of restoring synaptic function in a subject in need thereof by administering to the subject any one of the compounds of Formula I or a pharmaceutically acceptable salt thereof, or a pharmaceutical composition or a dosage form comprising it.

In another embodiment of the first to fourth aspects of the invention, the compounds here disclosed are sGC stimulators that may be useful in the prevention and/or treatment of diseases and disorders characterized by downregulated neurotransmitters. One embodiment of the invention is a method of normalizing neurotransmitter in a subject in need thereof by administering to the subject any one of the compounds of Formula I or a pharmaceutically acceptable salt thereof, or a pharmaceutical composition or a dosage form comprising it.

In another embodiment of the first to fourth aspects of the invention, the compounds here disclosed are sGC stimulators that may be useful in the prevention and/or treatment of diseases and disorders characterized by impaired cerebral blood flow. One embodiment of the invention is a method of restoring cerebral blood flow in a subject in need thereof by administering to the subject any one of the compounds of Formula I or a pharmaceutically acceptable salt thereof, or a pharmaceutical composition or a dosage form comprising it.

In another embodiment of the first to fourth aspects of the invention, the compounds here disclosed are sGC stimulators that may be useful in the prevention and/or treatment of diseases and disorders characterized by increased neurodegeneration. One embodiment of the invention is a method of decreasing neurodegeneration in a subject in need thereof by administering to the subject any one of the compounds of Formula I or a pharmaceutically acceptable salt thereof, or a pharmaceutical composition or a dosage form comprising it.

In another embodiment of the first to fourth aspects of the invention, the compounds here disclosed are sGC stimulators that may be useful in the prevention and/or treatment of diseases and disorders characterized by cognition impairment. One embodiment of the invention is a method of improving cognition in a subject in need thereof by administering to the subject any one of the compounds of Formula I or a pharmaceutically acceptable salt thereof, or a pharmaceutical composition or a dosage form comprising it. In some embodiments, the treatment improves memory. In other embodiments, the treatment improves attention. In other embodiments, the treatment improves executive function.

In another embodiment of the first to fourth aspects of the invention, the compounds here disclosed are sGC stimulators are neuroprotective. In particular, the compounds of Formula I or a pharmaceutically acceptable salt thereof, or a pharmaceutical composition or a dosage form comprising it may be useful protect the neurons in a subject in need thereof.

In some embodiments, the CNS disease, health condition or disorder is selected from Alzheimer's disease (AD), vascular dementia (VD), vascular cognitive impairment, mixed dementia, Binswanger's dementia (subcortical arteriosclerotic encephalopathy), cerebral autosomal-dominant arteriopathy with subcortical infarcts and leukoencephalopathy (CADASIL or CADASIL syndrome), frontotemporal lobar degeneration or dementia (FTD), asymptomatic neurocognitive impairment (ANI), subjective cognitive impairment or decline (SCD), cognitive ageing, minor neurocognitive disorder (MND), HIV-associated dementia (HAD) (also called AIDS dementia complex [ADC] or HIV encephalopathy), Lewy body dementia, pre-senile dementia or mild cognitive impairment (MCI)

In other embodiments, the disease, health condition or disorder is a CNS disorder or condition selected from sleep wake disorders, and neurological abnormalities associated with Sneddon's syndrome.

In other embodiments, the disease, health condition or disorder is a CNS disorder or condition selected from Alzheimer's disease or pre-Alzheimer's disease, mild to moderate Alzheimer's disease or moderate to severe Alzheimer's disease.

In other embodiments, the CNS disease is selected from glaucoma, Huntington's disease (or Huntington's chorea, HD), multiple sclerosis (MS), multiple system atrophy (MSA), Parkinson's disease (PD), Parkinsonism Plus, spinocerebellar ataxias (SCA), Steel-Richardson-Olszewski disease (progressive supranuclear palsy), amyotrophic lateral sclerosis (ALS or Lou Gehrig's disease) or Down's syndrome.

In other embodiments, the CNS disease is selected from attention deficit disorder (ADD) and attention deficit hyperactivity disorder (ADHD).

In other embodiments the CNS disorder is traumatic (closed or open) penetrating head injuries, traumatic brain injury (TBI), nontraumatic stroke (in particular, ischemic stroke), aneurism, hypoxia, or other injuries to the brain.

In other embodiments, the CNS disorder is a psychiatric, mental, mood or affective disorder selected from a bipolar disorder, schizophrenia, general psychosis, drug-induced psychosis, a delusional disorder, a schizoaffective disorder, obsessive compulsive disorder (OCD), a depressive disorder, an anxiety disorder, a panic disorder, or post-traumatic stress disorder (PTSD).

In other embodiments, the CNS disease or disorder is selected from a dystonia, including for example, generalized, focal, segmental, sexual, intermediate, genetic/primary dystonia or acute dystonic reaction; or a dyskinesia, including for example, acute, chronic/tardive, and non-motor and levo-dopa induced dyskinesia (LID).

In other embodiments, the CNS disease or disorder is selected from disorders characterized by a relative reduction in synaptic plasticity and synaptic processes including, for example, Fragile X, Rhett's disorder, Williams syndrome, Renpenning's syndrome, autism spectrum disorders (ASD), autism, Asperger's syndrome, pervasive development disorder or childhood disintegrative disorder.

In other embodiments, the CNS disorder is selected from chemo brain, levo-dopa induced addictive behavior, alcoholism, narcotic dependence (including but not limited to amphetamine, opiates or other substances) and substance abuse.

In one embodiment the CNS disease is cognitive impairment or dysfunction resulting from brain injuries, psychiatric disorders, neurodevelopmental disorders or neurodegenerative disorders.

In some embodiments of the methods and uses of the invention, cognitive impairment, either as MCI or dementia, is associated with Alzheimer's disease (AD), vascular dementia, mixed dementia, AD with vascular pathology (ADv), cerebral infarction, cerebral ischemia, stroke, head injury, traumatic head injury, learning disabilities, autism, attention deficit disorder, depression, spinocerebellar ataxia, Lewy body dementia, dementia with frontal lobe degeneration, Pick's syndrome, Parkinson's disease, progressive nuclear palsy, dementia with corticobasal degeneration, amyotrophic lateral sclerosis (ALS), Huntington's disease, demyelination diseases, multiple sclerosis (MS), thalamic degeneration, Creutzfeldt-Jakob dementia, HIV-dementia, schizophrenia, Korsakoff psychosis, post-operative cognitive decline in the elderly, bipolar disorder or mitochondrial disease. In other embodiments, cognitive impairment is associated with sickle cell disease.

In some embodiments of the methods and uses of the invention, MCI, dementia, sub-clinical cognitive impairment, or SCD is associated with cognitive aging, post-operative cognitive decline, medication side-effects, metabolic imbalances, hormonal problems, vitamin or nutrient deficiencies, delirium, psychiatric illness, damage to brain neurons due to an injury (for example in stroke or other cerebral vessel diseases or due to a traumatic brain injury), early stages of a neurodegenerative process, exposure to toxins, or viral or bacterial infections.

In other embodiments, the compounds here disclosed are sGC stimulators that may be useful in the prevention and/or treatment of orphan pain indications. One embodiment of the invention is a method of treating an orphan pain indication in a subject in need thereof by administering to the subject any one of the compounds of Formula I or a pharmaceutically acceptable salt thereof, or a pharmaceutical composition or a dosage form comprising it. In particular, the orphan pain indication is selected from Acetazolamide-responsive myotonia, Autoerythrocyte sensitization syndrome, Autosomal dominant Charcot-Marie-Tooth disease type 2V, Autosomal dominant intermediate Charcot-Marie-Tooth disease with neuropathic pain, Autosomal recessive limb-girdle muscular dystrophy type 2A, Channelopathy-associated congenital insensitivity to pain, Chronic pain requiring intraspinal analgesia, Complex regional pain syndrome, Complex regional pain syndrome type 1, Complex regional pain syndrome type 2, Congenital insensitivity to pain with hyperhidrosis, Congenital insensitivity to pain with severe intellectual disability, Congenital insensitivity to pain-hypohidrosis syndrome, Diffuse palmoplantar keratoderma with painful fissures, Familial episodic pain syndrome, Familial episodic pain syndrome with predominantly lower limb involvement, Familial episodic pain syndrome with predominantly upper body involvement, Hereditary painful callosities, Hereditary sensory and autonomic neuropathy type 4, Hereditary sensory and autonomic neuropathy type 5, Hereditary sensory and autonomic neuropathy type 7, Interstitial cystitis, Painful orbital and systemic neurofibromas-marfanoid habitus syndrome, Paroxysmal extreme pain disorder, Persistent idiopathic facial pain, Qualitative or quantitative defects of calpain, and Tolosa-Hunt syndrome.

In other embodiments, the CNS disorder is neuropathic pain. In some embodiment the pain is neuropathic pain associated with a CNS disease.

In further embodiments, the disease or condition is selected from acute pain, central pain syndrome, chemotherapy induced neuropathy, diabetic neuropathy, fibromyalgia, inflammatory pain, painful diabetic peripheral neuropathy, post-operative pain, tonic pain, and visceral pain.

In other embodiments, the compounds here disclosed are sGC stimulators that may be useful in the prevention and/or treatment of altitude (mountain) sickness, cerebral small vessel disease, cerebral vasculitis, cerebral vasospasm, hepatic encephalopathy, moyamoya, Parkinson's Dysphagia, ataxia telangliectasia, autism spectrum disorder, chronic fatigue, chronic traumatic encephalopathy (CTE), cognitive impairment associated with diabetes, cognitive impairment associated with multiple sclerosis, cognitive impairment associated with obstructive sleep apnea, cognitive impairment associated with schizophrenia (CIAS), cognitive impairment associated with sickle cell disease, concussion, retinopathy, diabetic retinopathy (including proliferative and non-proliferative), dysphagia.

In other embodiments, the compounds here disclosed are sGC stimulators that may be useful in the prevention and/or treatment of eye fibrosis, Fabry Disease, Gaucher Disease, glioblastoma, brain inflammation caused by cerebral malaria (SoC), brain inflammation caused by infectious disease, intellectual disability, myopic choroidal neovascularization, neuromyelitis optica, neuropathic pain with multiple sclerosis, neuropathic pain with shingles (herpes zoster), neuropathic pain with spine surgery, Parkinson's Dementia, peripheral and autonomic neuropathies, peripheral retinal degeneration, post-traumatic stress syndrome, post herpetic neuralgia, post-operative dementia, proliferative vitroretinopathy, radiation induced brain fibrosis, radiculopathy, refractory epilepsy, retinal vein occlusion, spinal cord injury, spinal muscular atrophy, spinal subluxations. tauopathies, and wet age-related macular degeneration.

The CNS diseases that may benefit from treatment with an sGC stimulator of the invention are those CNS diseases wherein an increase in the concentration of NO or an increase in the concentration of cGMP or both, or an upregulation of the NO-sGC-cGMP pathway might be desirable.

The compounds described herein, as well as pharmaceutically acceptable salts thereof, as stimulators of sGC that are able to cross the blood-brain barrier (BBB), are useful in the prevention and/or treatment of CNS diseases, conditions and disorders which can benefit from sGC stimulation in the brain.

In some embodiments, the compounds of the invention are able to stimulate sGC in the brain without producing a large blood pressure (BP) reduction in the patient. In some embodiments, the compound reduces the BP in the patient an average of less than 5 mm Hg for a dose that results in desired CNS effects. In other embodiments, an average of less than 10 mm Hg. In other embodiments, the reduction of BP in the patient is not clinically significant. In still other embodiments, the methods and uses of the invention do not result in a significant incidence of adverse events (AEs) associated with symptomatic hypotension when the patient is treated for a CNS disease.

As used herein, a dose that does not “result in a significant incidence of adverse events (AEs) associated with symptomatic hypotension” is one that does not result in excessive orthostatic hypotension, excessive dizziness, excessive postural dizziness, excessive pre-syncope, or excessive syncope in patients. Excessive orthostatic hypotension, excessive dizziness, excessive postural dizziness, excessive pre-syncope, or excessive syncope in patients are those that would warrant discontinuation of treatment by the patient or a recommendation of discontinuation by the practitioner.

In some embodiments the BP is measured as systolic BP. In other embodiments, as diastolic BP. In other embodiments as mean arterial pressure (MAP). In other embodiments BP is ambulatory measured BP. In other embodiments, BP is cuff-measured BP. In some embodiments, the measurement of BP is with the patient in the supine position. In other embodiments, the patient is in a sitting position.

The term “disease”, as used herein refers to any deviation from or interruption of the normal structure or function of any body part, organ, or system that is manifested by a characteristic set of symptoms and signs and whose etiology, pathology, and prognosis may be known or unknown. The term disease encompasses other related terms such as disorder and condition (or medical condition) as well as syndromes, which are defined as a combination of symptoms resulting from a single cause or so commonly occurring together as to constitute a distinct clinical picture. In some embodiments, the term disease refers to an sGC, cGMP and/or NO mediated medical or pathological disease. In still other embodiments, the term disease refers to an sGC, cGMP and/or NO mediated medical or pathological condition affecting the CNS. As used herein, a “CNS disease, health condition or disorder” means the same as a CNS disease, a CNS health condition or a CNS disorder, and the terms disease, condition and disorder in this disclosure, independently on how they are used, all refer to those affecting the CNS only (central nervous system).

“Treat”, “treating” or “treatment” with regard to a disorder, disease, condition, symptom or syndrome, refers to abrogating or improving the cause and/or the effects (i.e., the symptoms, physiological, physical, psychological, emotional or any other clinical manifestations, observations or measurements, or improving pathological assessments) of the disorder, disease, condition or syndrome.

As used herein, the terms “treat”, “treatment” and “treating” also refer to the delay or amelioration or prevention of the progression (i.e. the known or expected progression of the disease), severity and/or duration of the disease or delay or amelioration or prevention of the progression of one or more symptoms, clinical manifestations, observations or measurements, or preventing or slowing down the negative progression of pathological assessments (i.e. “managing” without “curing” the condition), resulting from the administration of one or more therapies.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In some embodiments, the subject is a non-human animal such as a farm animal (e.g., a horse, cow, pig or sheep), or a companion animal or pet (e.g., a dog, cat, mice, rats, hamsters, gerbils, guinea pig or rabbit). In some embodiments, the subject is a human.

The invention also provides a method for treating one of the above diseases in a subject, comprising administering a therapeutically effective amount of a compound of Formula I, or a pharmaceutically acceptable salt thereof, to the subject in need of the treatment. Alternatively, the invention provides the use of a compound of Formula I, or a pharmaceutically acceptable salt thereof, in the treatment of one of these diseases in a subject in need of the treatment. Also included in the invention is the use of a compound of Formula I, or a pharmaceutically acceptable salt thereof, for the manufacture of medicament for treating one of the above diseases in a subject in need of the treatment. The invention further provides a method of making or manufacturing a medicament useful for treating one of these diseases comprising using a compound of Formula I, or a pharmaceutically acceptable salt thereof.

The term “biological sample”, as used herein, refers to an in vitro or ex vivo sample, and includes, without limitation, cell cultures or extracts thereof; biopsied material obtained from a mammal or extracts thereof; blood, saliva, urine, faeces, semen, tears, lymphatic fluid, ocular fluid, vitreous humor, cerebrospinal fluid (CSF), or other body fluids or extracts thereof.

In other embodiments, the invention provides a method of stimulating sGC activity in a biological sample, comprising contacting said biological sample with a compound or composition of the invention. Use of a sGC stimulator in a biological sample is useful for a variety of purposes known to one of skill in the art. Examples of such purposes include, without limitation, biological assays and biological specimen storage.

### Combination Therapies

The compounds and pharmaceutical compositions described herein can be used alone or in combination therapy for the treatment of a disease mediated, regulated or influenced by sGC, cGMP and/or NO.

As used herein, the terms “in combination” (as in the sentence “in combination therapy”) or “co-administration” can be used interchangeably to refer to the use of more than one therapy. The use of the terms does not restrict the order in which therapies are administered to a subject.

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When used in combination therapy with other agents, a “therapeutically effective amount” of the compounds and pharmaceutical compositions described herein and of the other agent or agents will depend on the type of drug used. Suitable dosages are known for approved agents and can be adjusted by the skilled artisan according to the condition of the subject, the type of condition(s) being treated, and the amount of a compound described herein being used. In cases where no amount is expressly noted, an effective amount should be assumed.

In some embodiments, co-administration or combination therapy encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such co administration also encompasses use of each compound in a sequential manner in either order.

When co-administration involves the separate administration of a first amount of a compound of Formula I and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of Formula I and the second therapeutic agent can be administered in any order within about 24 hours of each other, within about 16 hours of each other, within about 8 hours of each other, within about 4 hours of each other, within about 1 hour of each other or within about 30 minutes of each other.

Examples of other therapeutic agents that may be combined with a compound of Formula I, or a pharmaceutically acceptable salt thereof, either administered separately or in the same pharmaceutical composition include, but are not limited to:
(1) Endothelium-derived releasing factor (EDRF) or NO gas.
(2) NO donors including, but not limited to: a nitrosothiol, a nitrite, a sydnonimine, a NONOate, a N-nitrosamine, a N-hydroxyl nitrosamine, a nitrosimine, nitrotyrosine, a diazetine dioxide, an oxatriazole 5-imine, an oxime, a hydroxylamine, a N-hydroxyguanidine, a hydroxyurea or a furoxan. Some examples of these types of compounds include: glyceryl trinitrate (also known as GTN, nitroglycerin, nitroglycerine, and trinitrogylcerin), the nitrate ester of glycerol; sodiumnitroprusside (SNP), wherein a molecule of nitric oxide is coordinated to iron metal forming a square bipyramidal complex; 3-morpholinosydnonimine (SIN-1), a zwitterionic compound formed by combination of a morpholine and a sydnonimine; S-nitroso-N-acetylpenicillamine (SNAP), an N-acetylated amino acid derivative with a nitrosothiol functional group; diethylenetriamine/NO (DETA/NO), a compound of nitric oxide covalently linked to diethylenetriamine; an m-nitroxymethyl phenyl ester of acetyl salicylic acid. More specific examples of some of these classes of NO donors include: the classic nitrovasodilators, such as organic nitrate and nitrite esters, including nitroglycerin, amyl nitrite, isosorbide dinitrate, isosorbide 5-mononitrate, and nicorandil; isosorbide; 3-morpholinosydnonimine; linsidomine chlorohydrate (“SIN-1”); S-nitroso-N-acetylpenicillamine (“SNAP”); S-nitrosoglutathione (GSNO), sodium nitroprusside, S-nitrosoglutathione mono-ethyl-ester (GSNO-ester), 6-(2-hydroxy-1-methyl-nitrosohydrazino)-N-methyl-1-hexanamine or diethylamine NONOate.
(3) Other substances that enhance cGMP concentrations, including, but not limited toprotoporphyrin IX, arachidonic acid and phenyl hydrazine derivatives.
(4) Nitric Oxide Synthase substrates, including, but not limited to L-arginine, n-hydroxyguanidine based analogs, such as N[G]-hydroxy-L-arginine (NOHA), 1-(3, 4-dimethoxy-2-chlorobenzylideneamino)-3-hydroxyguanidine, and PR5 (1-(3, 4-dimethoxy-2-chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluoromethyl) propylguanidine.
(5) Compounds which enhance eNOS transcription.
(6) NO independent heme-independent sGC activators, including, but not limited to BAY 58-2667 (described in patent publication DE19943635); HMR-1766 (ataciguat, described in patent publication WO2000002851); S 3448 (2-(4-chloro-phenylsulfonylamino)-4,5-dimethoxy-N-(4-(thiomorpholine-4-sulfonyl)-phenyl)-benzamide (described in patent publications DE19830430 and WO2000002851); and HMR-1069 (from Sanofi-Aventis).
(7) Heme-dependent, NO-independent sGC stimulators including, but not limited to YC-1 (see patent publications EP667345 and DE19744026); riociguat (BAY 63-2521, Adempas®, described in DE19834044); nelociguat (BAY 60-4552, described in WO 2003095451); vericiguat (BAY 1021189, described in U.S. Pat. No. 8,420,656); BAY 41-2272 (described in DE19834047 and DE19942809); BAY 41-8543 (described in DE19834044); etriciguat (described in WO 2003086407); CFM-1571 (described in patent publication WO2000027394); A-344905, its acrylamide analogue A-350619 and the aminopyrimidine analogue A-778935;
other sGC stimulators described in one of publications US20090209556, U.S. Pat. No. 8,455,638, US20110118282 (WO2009032249), US20100292192, US20110201621, U.S. Pat. Nos. 7,947,664, 8,053,455 (WO2009094242), US20100216764, U.S. Pat. No. 8,507,512, (WO2010099054) US20110218202 (WO2010065275), US20130012511 (WO2011119518), US20130072492 (WO2011149921), US20130210798 (WO2012058132), and Tetrahedron Letters (2003), 44(48): 8661-8663; and IW1973 (praliciguat), IW1701 (olinciguat) and CY6463 (previously IW-6463).
(8) Compounds that inhibit the degradation of cGMP and/or cAMP, including, but not limited to:
PDE1 inhibitors, PDE2 inhibitors, PDE-3 inhibitors such as, for example, amrinone, milrinone, enoximone, vesnarinone, pimobendan, and olprinone, PDE4 inhibitors, such as, for example, rolumilast, PDE5 inhibitors, such as, for example, sildenafil and related agents such as avanafil, lodenafil, mirodenafil, sildenafil citrate, tadalafil, vardenafil and udenafil; alprostadil; dipyridamole and PF-00489791; PDE6 inhibitors, PDE9 inhibitors, such as, for example, PF-04447943, PDE10 inhibitors such as, for example, PF-02545920 (PF-10), and PDE11 inhibitors.
(9) Anticoagulants, including but not limited to:
coumarines (Vitamin K antagonists) such as warfarin, cenocoumarol, phenprocoumon and phenindione;
heparin and derivatives such as low molecular weight heparin, fondaparinux and idraparinux;
direct thrombin inhibitors such as argatroban, lepirudin, bivalirudin, dabigatran and ximelagatran; and
tissue-plasminogen activators, used to dissolve clots and unblock arteries, such as alteplase.
(10) Antiplatelet drugs, including, but not limited to topidogrel, ticlopidine, dipyridamoleand aspirin.
(11) Supplemental oxygen therapy.
(12) Alpha-1-adrenoceptor antagonists, including, but not limited to prazosin, indoramin, urapidil, bunazosin, terazosin and doxazosin; atrial natriuretic peptide (ANP), ethanol, histamine-inducers, tetrahydrocannabinol (THC) and papaverine.
(13) Bronchodilators, including, but not limited to:
short acting β₂ agonists, such as albutamol or albuterol and terbutaline;
long acting β₂ agonists (LABAs) such as salmeterol and formoterol;
anticholinergics such as pratropium and tiotropium; and
theophylline, a bronchodilator and phosphodiesterase inhibitor.
(14) Corticosteroids, including, but not limited to beclomethasone, methylprednisolone, betamethasone, prednisone, prednisolone, triamcinolone, dexamethasone, fluticasone, flunisolide, hydrocortisone, and corticosteroid analogs such as budesonide.
(15) Dietary supplements, including but not limited to omega-3 oils; folic acid, niacin, zinc, copper, Korean red ginseng root, ginkgo, pine bark, Tribulus terrestris, arginine, Avena sativa, horny goat weed, maca root, muira puama, saw palmetto, and Swedish flower pollen; vitamin C, Vitamin E, Vitamin K2; testosterone supplements, testosterone transdermal patch; zoraxel, naltrexone, bremelanotide and melanotan II.
(16) PGD2 receptor antagonists.
(17) Immunosuppressants, including, but not limited to cyclosporine, tacrolimus, rapamycin and other FK-506 type immunosuppressants, mycophenolate, mycophenolate mofetil.
(18) Non-steroidal anti-asthmatics, including, but not limited to:
β₂-agonists like terbutaline, metaproterenol, fenoterol, isoetharine, albuterol, salmeterol, bitolterol and pirbuterol;
β₂-agonist-corticosteroid combinations such as salmeterol-fluticasone, formoterol-budesonide, theophylline, cromolyn, cromolyn sodium, nedocromil, atropine, ipratropium, ipratropium bromide; and
leukotriene biosynthesis inhibitors such as zileuton or veliflapon.
(19) Non-steroidal anti-inflammatory agents (NSAIDs), including, but not limited to: propionic acid derivatives like alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen;
acetic acid derivatives such as indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac;
fenamic acid derivatives such as flufenamic acid, meclofenamic acid, mefenamic acid, niflumic acid and tolfenamic acid;
biphenylcarboxylic acid derivatives such as diflunisal and flufenisal;
oxicams such as isoxicam, piroxicam, sudoxicam and tenoxican;
salicylates such as acetyl salicylic acid and sulfasalazine; and
pyrazolones such as apazone, bezpiperylon, feprazone, mofebutazone, oxyphenbutazone and phenylbutazone.
(20) Cyclooxygenase-2 (COX-2) inhibitors, included, but not limited to celecoxib, rofecoxib, valdecoxib, etoricoxib, parecoxib and lumiracoxib; opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine.
(21) Adrenergic neuron blockers, including, but not limited to guanethidine and guanadrel.
(22) Imidazoline I-1 receptor agonists, including, but not limited to rimenidine dihydrogen phosphate and moxonidine hydrochloride hydrate.
(23) Potassium channel activators, including, but not limited to pinacidil.
(24) Dopamine D1 agonists, including, but not limited to fenoldopam mesilate; other dopamine agonists such as ibopamine, dopexamine and docarpamine.
(25) 5-HT2 antagonists, including, but not limited to ketanserin.
(26) Vasopressin antagonists, including, but not limited to tolvaptan.
(27) Calcium channel sensitizers, including, but not limited to levosimendan or activators such as nicorandil.
(28) Adenylate cyclase activators, including, but not limited to colforsin dapropate hydrochloride.
(29) Positive inotropic agents, including, but not limited to digoxin and metildigoxin; metabolic cardiotonic agents such as ubidecarenone; brain natriuretic peptides such as nesiritide.
(30) Drugs used for the treatment of erectile dysfunction, including, but not limited to alprostadil, aviptadil, and phentolamine mesilate.
(31) Drugs used for the treatment of Alzheimer's disease and dementias, including but not limited to:
acetyl cholinesterase inhibitors such as galantamine, rivastigmine, donepezil and tacrine; and
NMDA receptor antagonists such as mnemantine; and
oxidoreductase inhibitors such as idebenone.
(32) Psychiatric medications, including, but not limited to:
ziprasidone, risperidone, olanzapine, valproate;
dopamine D4 receptor antagonists such as clozapine;
dopamine D2 receptor antagonists such as nemonapride;
mixed dopamine D1/D2 receptor antagonists such as zuclopenthixol;
GABA A receptor modulators such as carbamazepine;
sodium channel inhibitors such as lamotrigine;
monoamine oxidase inhibitors such as moclobemide and indeloxazine; and
primavanserin, and perospirone.
(33) Drugs used for the treatment of movement disorders or symptoms, including, but not limited to:
catechol-O-methyl transferase inhibitors such as entacapone;
monoamine oxidase B inhibitors such as selegiline;
dopamine receptor modulators such as levodopa;
dopamine D3 receptor agonists such as pramipexole;
decarboxylase inhibitors such as carbidopa;
other dopamine receptor agonists such as pergolide, ropinirole, cabergoline;
ritigonide, istradefylline, talipexole; zonisamide and safinamide; and
synaptic vesicular amine transporter inhibitors such as tetrabenazine.
(34) Drugs used for the treatment of mood or affective disorders or OCD such as the following types:
tricyclic antidepressants such as amitriptyline, desipramine, imipramine, amoxapine, nortriptyline, doxepin and clomipramine;
selective serotonin reuptake inhibitors (SSRIs) such as paroxetine, fluoxetine, sertraline, trazodone and citralopram;
atypical antidepressants such as agomelatine;
selective norepinephrine reuptake inhibitors (SNRIs) such as venlafaxine, reboxetine and atomoxetine; dopaminergic antidepressants such as bupropion and amineptine.
(35) Drugs for the enhancement of synaptic plasticity, including, but not limited to:
nicotinic receptor antagonists such as mecamylamine; and
mixed 5-HT, dopamine and norepinephrine receptor agonists such as lurasidone.
(36) Drugs used for the treatment of ADHD such as amphetamine; 5-HT receptor modulators such as vortioxetine and alpha-2 adrenoceptor agonists such as clonidine.
(37) Nitric oxide synthase cofactors, including, but not limited to tetrahydrobiopterin, dihydrobiopterin and sapropterin.
(38) Blood glucose lowering medications (also referred as glycemic control medications or antidiabetic medications) including, but not limited to:
biguanides such as metformin;
sulfonylureas such as glyburide, glybenclamide, glipizide, gliclazide, gliquidone, glimepiride, atorvastatin calcium combined with glimerpiride, meglinatide, tolbutamide, chlorpropamide, acetohexamide, and tolazimide;
alpha-glucosidase inhibitors such as acarbose, epalrestat, voglibose, and miglitol;
insulin secretagoges such as repaglinide, mitiglinide and nateglinide;
thiazolidinediones such as rosiglitazone, troglitazone, ciglitazone, pioglitazone, englitazone, lobeglitazone sulfate and balaglitazone;
DPP-4 inhibitors (or DPP-IV inhibitors) such as sitagliptin, vildagliptin, saxagliptin, alogliptin, linagliptin, alogliptin benzoate combined with metformin or metformin hydrochloride, anagliptin, teneligliptin, atorvastatin calcium and glimepiride, empagliflozin combined with linagliptin, gemigliptin, sitagliptin phosphate monohydrate combined with pioglitazone hydrochloride, sitagliptin combined with pioglitazone, sitagliptin combined with atorvastatin calcium, and (2S,4S)-1-[2-(1,1-dimethyl-3-oxo-3-pyrrolidin-1-yl-propylamino)acetyl]-4-fluoro-pyrrolidine-2-carbonitrile (DBPR-108);
GLP-1 receptor agonists or incretin mimetics such as exenatide, dulaglutide, liraglutide, semaglutide, lixisenatide, lixisenatide combined with insulin glargine, albiglutide and pegapamodutide (TT-401), LY3298176 (dual glucose-dependent insulinotropic polypeptide (GIP) and GLP-1 receptor agonist);
SGLT2 inhibitors (SGLT2is) such as empagliflozin, empaglifozin combined with linagliptin, empagliflozin combined with metformin, ipragliflozin, ipragliflozin L-proline, tofogliflozin, sergliflozin etabonate, remogliflozin etabonate, ertugliflozin, ertugliflozin combined with sitagliptin, ertugliflozin combined with metformin, sotagliflozin, canagliflozin, canagliflozin combined with metformin or metformin hydrochloride, dapagliflozin, dapagliflozin combined with metformin or metformin hydrochloride and luseoglifozin, dapagliflozin combined with saxagliptin;
SGLT1 inhibitors or combinations of SGLT1 and SGLT2 inhibitors such as sotagliflozin;
insulin therapy such one of the many types of insulin, like insulin glulisine, insulin degludec, insulin lispro, insulin aspart, insulin glargine, insulin detemir, insulin isophane, insulin mixtard (human insulin containing both fast-acting (soluble) and long-acting (isophane) insulin, insulin degludec combined with insulin aspart, insulin human (rDNA origin) inhalation powder, recombinant human insulin, hepatic-directed vesicle insulin, insulin tregopi (IN-105), insulin degludec combined with liraglutide, insulin peglispro (LY-2605541) and nodlin; and
tolimidone (a lyn kinase activator).
(39) Blood pressure lowering medications (also known as anti-hypertensive medications), including, but not limited to:
diuretics such as thiazide diuretics, chlorothiazide, chlorthalidone, hydrochlorothiazide, bendroflumethiazide, cyclopenthiazide, methyclothiazide, polythiazide, quinethazone, xipamide, metolazone, indapamide, cicletanine, furosemide, toresamide, amiloride, spironolactone, canrenoate potassium, eplerenone, triamterene, acetazolamid and carperitide;
beta blockers such as acebutolol, atenolol, metoprolol, and nebivolol;
angiotensin-converting enzyme (ACE) inhibitors such as sulfhydryl-containing agents (for example, captopril, zofenopril), dicarboxylate-containing agents (for example, enalapril, quinapril, ramipril, perindopril, lisinopropil, and benazepril), phosphonate-containing agents (for example fosinopril), naturally occurring ACE inhibitors (for example, casokinins, lactokinins, lactotripeptides Val-Pro-Pro and Ile-Pro-Pro), alacepril, delapril, cilazapril, imidapril, temocapril, moexipril, lisinopril, combinations of lisinopril with hydrochlorothiazide, trandolapril and spirapril;
angiotensin II receptor blockers (ARBs) such as candesartan, losartan, losanan potassium-hydrochlorothiazide, valsartan, candesartan cilexetil, eprosaran, irbesartan, telmisartan, olmesartan medoxomil (or olmesartan), azilsartan medoxomil, azilsartan, amlodipine besylate combined with irbesartan, azilsartan combined with amlodipine besilate, cilnidipine combined with valsartan, fimasartan, irbesartan combined with atorvastatin, irbesartan combined with trichlormethiazide, losartan potassium combined with hydrochlorothiazide and/or amlodipine besylate, pratosartan, atorvastatin calcium combined with losartan potassium, nifedipine and candesartan cilexetil, sacubitril combined with valsartan or LCZ-696, angiotensin AT2 antagonist and TAK-591 and olmesartan medoxomil;
endothelin Receptor antagonists (ERAs) such as atrasentan, bosentan, sitaxentan, ambrisentan, actelion-1 (macitentan), Cyclo(D-trp-D-asp-L-pro-D-val-L-leu) (BQ-123), sparsentan and tezosentan disodium;
mineralocorticoid receptor antagonists (MRAs) such as spironolactone, amiloride hydrochloride combined with spironolactone, apararenone or MT-3995, eplerenone, and finerenone (BAY-94-8862);
calcium channel blockers such as amlodipine, aranidipine, azelnidipine, barnidipine, benidipine, cilnidipine, clevidipine, diltiazem, efonidipine, felodipine, lacidipine, lercanidipine, manidipine, nicardipine, nifedipine, nilvadipine, nimodipine, nisoldipine, nitrendipine, pranidipine, isradipine, verapamil, gallopamil, diltiazem, mibefradil, bepridil, fluspirilene and fendiline;
renin inhibitors such as aliskiren;
alpha blockers such as doxazosin and prazosin;
alpha-beta blockers such as carvedilol and labetalol;
central-acting agents such as clonidine, guanfacine and methyldopa;
vasodilators such as nitroglycerine, hydralazine and minoxidil; and aldosterone antagonists such as finerenone, spironolactone and eplerenone.
(40) Anti-hyperlipideric medications, including but not limited to:
statins such as atorvastatin fluvastatin, lovastatin, pitavastatin, pravastatin, rosuvastatin, and simvastatin;
combinations of statins with another agent such as amlodipine/atorvastatin, aspirin/pravastatin, ezetimibe/simvastatin, niacin/simvastatin, lovastatin/niacin, simvastatin/sitagliptin and atorvastatin/ezetimibe;
fibrates or fibric acid derivatives. Examples include, but are not limited to, fenofibrate, gemfibrozil, bezafibrate, ciprofibrate, clinofibrate and clofibrate;
niacin (or nicotinic acid);
bile acid sequestrants such as cholestyramine, colesevelam, colestilan and colestipol;
ezetimibe, lomitapide, phytosterols or orlistat; and
PCSK9 inhibitors such as alirocumab and evolocumab;
(41) Neprilysin inhibitors (also known as endopeptidase inhibitors or NEP inhibitors or enkephalinase inhibitors), including, but not limited to sacubitril, or the combination of sacubitril with valsartan; neprilysin inhibitors in development TD-1439 or TD-0714.
(42) Renoprotective drugs, including, but are not limited to:
bardoxolone;
ACE inhibitors such as captopril;
ARBs such as losartan or irbesartan;
SGLT2 inhibitors such as canagliflozin,
GLP1 receptor agonists;
MRAs such as finerenone;
ERAs such as atrasentan; and
apoptosis signal-regulating kinase 1 (ASK1) inhibitors such as selonsertib.
(43) Hydroxyurea (HU, hydroxycarbamide).
(44) Anti-sickling agents, including, but not limited to hydroxyurea, voxelotor or GBT-440.
(45) Anti-adhesion therapies, including, but not limited, to blocking antibodies to P-selectin, E-selectin, VLA-4, VCAM-1.
(46) Glutamine.
(47) Erythropoietin (EPO), also known as hematopoietin or hemopoietin, including all its forms such as exogenous erythropoietin, recombinant human erythropoietin (rhEPO) or other erythropoiesis-stimulating agents (ESA) two examples being epoetin alfa and epoetin beta.
(48) Antibiotics, including but not limited to:
penicilin and its derivatives, including, but not limited to penicillin, amoxicillin, ampicillin, azlocillin, cloxacillin, penicillin G, penicillin V, procaine penicillin or benzathine penicillin amongst others.
cephalosporins such as cephalexin, cefadroxil, cefaclor, cefuroxime and cefexime;
macrolides such as erythromycin, clarithromycin, azithromycin, and roxithromycin;
tetracycline and its derivatives such as demeclocycline, doxycycline, minocycline, oxytetracycline and tetracycline;
sulfonamides, including, but not limited to, mafenide, sulfacetamide, sulfadiazine, silver sulfadiazine, sulfadimethoxine, sulfamethizole, sulfamethoxazole, sulfasalazine, trimethoprim-sulfamethoxazole (Co-trimoxazole), and sulfisoxazole; and
quinolones, including, but not limited to ciprofloxacin, enoxacin, gatifloxacin, gemifloxacin, levofloxacin, lomefloxacin, moxifloxacin, ofloxacin, and nalidixic acid.
(49) FXR agonists, including but not limited to obeticholic acid, cenicriviroc, emricasan, GR-MD-02, selonsertib and elafibranor.
(50) Thyroid receptor-beta agonists, including, but not limited to MGL-3196.
(51) Acetyl-CoA carboxylase inhibitors, including but not limited to GS-0976.
(52) Treatments for mitochondrial disorders including, but not limited to, vitamins and supplements, including Coenzyme Q10; B complex vitamins, especially thiamine (B1) and riboflavin (B2); Alpha lipoic acid; L-carnitine (Carnitor); Creatine; Citrulline, and L-Arginine.
(53) Treatments for epilepsy or seizures including, but not limited to, phenytoin, valproic acid, phenobarbital, lamotrigine, carbamazepine, topiramate, oxcarbazepine, zonisamide, gabapentin, levetiracetam, pregabalin, clonazepam, lacosamide, rufinamide, and vigabatrin.

### Packaging and Kits

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for co therapy use, a pharmaceutically acceptable carrier, vehicle or diluent. The pharmaceutical composition comprising the compound described herein and the second pharmaceutical composition contained in the kit may be optionally combined in the same pharmaceutical composition.

### EXAMPLES

All references provided in the Examples are herein incorporated by reference. As used herein, all abbreviations, symbols and conventions are consistent with those used in the contemporary scientific literature. See, e.g. Janet S. Dodd, ed., The ACS Style Guide: A Manual for Authors and Editors, 2^{nd} Ed., Washington, D.C.: American Chemical Society, 1997, herein incorporated in its entirety by reference.

Various embodiments of the invention can be described in the text below.

Definitions of the abbreviations used in the Examples section is provided in the table below.

| Abbreviation | Word or Phrase |
|---|---|
| app. | Apparent |
| Conc. | Concentration |
| DMF | N,N-dimethylformamide |
| DMSO | Dimethylsulphoxide |
| DMSO-d6 | Deuterated Dimethylsulphoxide |
| dppf | 1.1′-bis(diphenylphosphino)ferrocene |
| dba | dibenzylideneacetone |
| ES+ | Positive electron spray ionization |
| EtOAc | ethyl acetate |
| equiv. | Equivalent |
| FA | Formic Acid |
| h | hour(s) |
| HEK | Human embryonic kidney |
| HPLC | high performance liquid chromatography |
| i-PrOH | Isopropyl alcohol |
| LC-MS | Liquid Chromatography- Mass Spectrometry |
| MeOH | Methanol |
| mL | Milliliter |
| MHz | Megahertz |
| MS | Mass Spectrometry |
| NMR | Nuclear Magnetic Resonance |
| No. | Compound Number |
| Ph | Phenyl |
| ppm | parts per million |
| RP- HPLC | reversed phase high performance liquid chromatography |
| TFA | Trifluoroacetic acid |
| THF | Tetrahydrofuran |

### Synthesis Section

### Example 1: Synthesis of Compounds of Formula I

The present invention also provides methods for synthesizing the compounds of Formula I, which represent another embodiment of this invention. Compounds of this invention of Formula I may be prepared according to the general and specific syntheses described herein, synthetic procedures reported in the chemical literature or methods known to a person of ordinary skill in the art. As could be appreciated by those of ordinary skill in the art, optimum reaction condition, which may be determined during the experimentation, may vary based on the reaction type and the specific reagents used in the reaction. As such, unless specifically described, the reaction conditions such as pressure, temperature, relative ratio of the reagents, solvent, and reaction time may be readily selected and modified, without undue experimentation, by a person of ordinary skill in the art.

Compounds and intermediates of this invention may be purified by purification methods known to a person of ordinary skill in the art. These methods include, but are not limited to, silica gel chromatography, recrystallization, reverse phase HPLC (RP-HPLC) and Supercritical Fluid Chromatography (SFC). Purification on RP-HPLC may be accomplished on a suitable reverse phase column (e.g., Waters XBridge OBD C18, 5 μm, 19×150 mm) using a suitable gradient selected from a range of 0% to 100% acetonitrile in water containing an additive such as 0.1% TFA or FA. Diastereomers may be separated by silica gel chromatography, RP-HPLC or chiral HPLC. Discrete enantiomers may be obtained from a mixture of enantiomers by resolution using a chiral HPLC. Reaction progress may be monitored by methods known to one of ordinary skill in the art such as thin layer chromatography, reverse phase HPLC, or tandem reverse phase HPLC-Mass Spectrometry (LC-MS).

Starting materials used in the syntheses described herein are available from commercial sources or may be prepared by a person of ordinary skill in the art using methods reported in the chemical literature or referenced herein.

The general methods described herein may be used to prepare compounds of Formula I and compounds of Formula I. The general and specific methods described herein are provided as illustrations of the enablement of the present invention. As such, they are not intended to impose any restrictions on the subject matter and the scope of the claimed compounds of this invention. All references provided in the Examples are herein incorporated by reference. As used herein, all abbreviations, symbols and conventions are consistent with those used in the contemporary scientific literature. See, e.g. G. M. Banik, G. Baysinger, P. V. Kamat, N. J. Pienta, eds., The ACS Guide to Scholarly Communication, Washington, D.C.: American Chemical Society, 2020 (https;//pub.acs.org/doi/book/10.1021/acsguide), herein incorporated in its entirety by reference.

### Example 1: Compounds Syntheses

Compounds herein disclosed can be made, for example, using the general procedure depicted below (General Procedure C) from the corresponding nitrile intermediates:

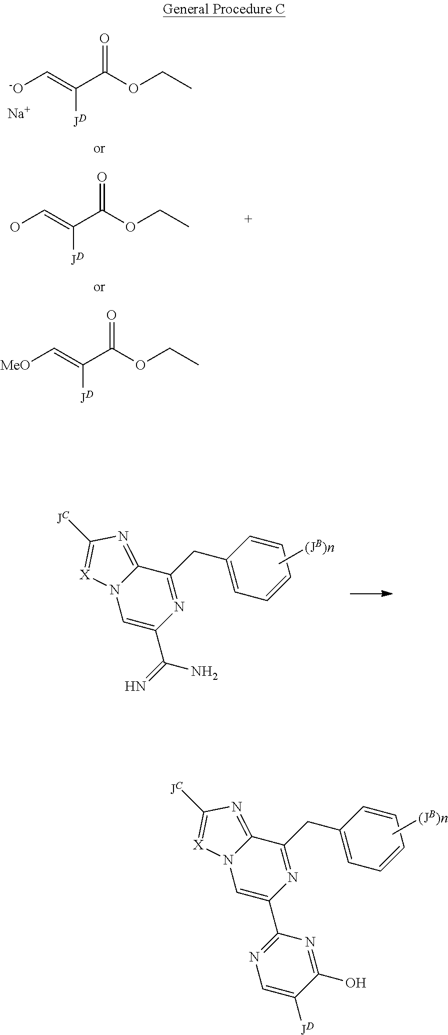

Compounds of the invention can be prepared by following similar procedures to those described herein via their corresponding nitriles. Nitriles with different patterns of substitution can be prepared by following the procedures described in WO2015187470, WO2016081668, WO2017197555, WO2017200825, WO2018/045276A1 and WO2019/126354A1.

The following nitrile intermediates were prepared according to literature procedures described in WO2018/045276A1 and WO2019/126354A1. The reaction conditions (such as reagents ratio, temperature and reaction time) and purification methods were modified as needed.
8-Benzylimidazo[1,2-a]pyrazine-6-carbonitrile;
8-(3-Fluorobenzyl)imidazo[1,2-a]pyrazine-6-carbonitrile;
8-(2-Fluorobenzyl)imidazo[1,2-a]pyrazine-6-carbonitrile;
8-(2,3-Difluorobenzyl)imidazo[1,2-a]pyrazine-6-carbonitrile;
8-(2,5-Difluorobenzyl)imidazo[1,2-a]pyrazine-6-carbonitrile;
8-(3-Fluoro-4-methylbenzyl)imidazo[1,2-a]pyrazine-6-carbonitrile;
8-(3,5-Difluorobenzyl)imidazo[1,2-a]pyrazine-6-carbonitrile;
8-(3,5-Difluoro-4-methylbenzyl)imidazo[1,2-a]pyrazine-6-carbonitrile;
8-(2,5-Difluoro-4-methylbenzyl)imidazo[1,2-a]pyrazine-6-carbonitrile;
8-(2,5-Difluorobenzyl)-[1,2,4]triazolo[1,5-a]pyrazine-6-carbonitrile;
8-(3-Fluorobenzyl)-[1,2,4]triazolo[1,5-a]pyrazine-6-carbonitrile;
8-(3,5-Difluorobenzyl)-[1,2,4]triazolo[1,5-a]pyrazine-6-carbonitrile;
8-(2,3-Difluorobenzyl)-[1,2,4]triazolo[1,5-a]pyrazine-6-carbonitrile;
8-(2,5-Difluoro-4-methylbenzyl)-[1,2,4]triazolo[1,5-a]pyrazine-6-carbonitrile.

The procedures for the synthesis of Compounds I-1 to I-20 are described below. The procedures for the synthesis of compounds I-20 to I-26 are described in patent application publication WO2019/126354.

### General Procedure a, as Applied to the Synthesis Compound I-1

The title compound was synthesized in 2 steps:

### Step 1: Synthesis of 8-(3-fluorobenzyl)imidazo[1,2-a]pyrazine-6-carboximidamide

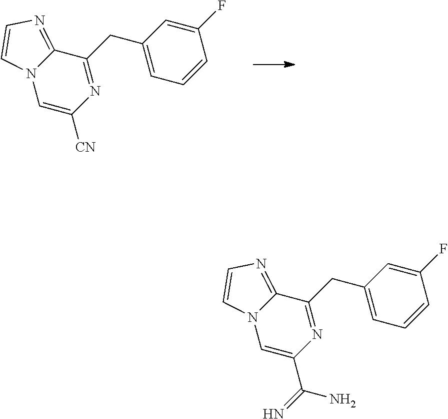 [SMILES: N#Cc1cn2ccnc2c(Cc2cccc(F)c2)n1 ; N=C(N)c1cn2ccnc2c(Cc2cccc(F)c2)n1]

To a solution of 8-(3-fluorobenzyl)imidazo[1,2-a]pyrazine-6-carbonitrile (220 mg, 0.87 mmol, 1.0 equiv.) in methanol (5.0 mL) was added a solution of 0.50 N sodium methanolate in methanol (0.17 mL, 0.087 mmol, 0.10 equiv.) (note: stoichiometric or excess amount of sodium methanolate could also be used). After 6 h of stirring at ambient temperature, ammonium chloride (280 mg, 5.2 mmol, 6.0 equiv.) was added and the reaction was allowed to stir for 16 h. The reaction mixture was concentrated in vacuo, diluted with half-saturated NaHCO₃ solution (20 mL) and extracted with 2×20 mL of CH₂Cl₂/iPrOH (5:1). The combined organic phases were dried over sodium sulfate, filtered, and concentrated to afford the crude product carboximidamide as a tan foam solid. It was used in the next step without further purification. LC/MS ES⁺ m/z=270.2 [M+H]⁺.

### Step 2: Synthesis of 5-fluoro-2-(8-(3-fluorobenzyl)imidazo[1,2-a]pyrazin-6-yl)pyrimidin-4-ol

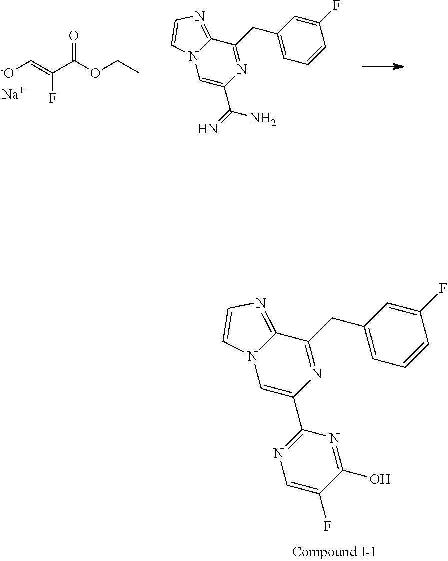

To a suspension of 8-(3-fluorobenzyl)imidazo[1,2-a]pyrazine-6-carboximidamide (210 mg, 0.79 mmol, 1.0 equiv.) in ethanol (7.0 mL) was added sodium (Z)-3-ethoxy-2-fluoro-3-oxoprop-1-en-1-olate (490 mg, 3.1 mmol, 4.0 equiv.). The reaction was heated in a sealed vial at 90° C. for 2.5h. After cooling to ambient temperature, 1.0 N aqueous HCl solution (3.1 mL, 3.1 mmol, 4.0 equiv.) was added. The resultant mixture was concentrated in vacuo, diluted with water (50 mL), adjusted to pH 6 with saturated NaHCO₃ solution, and extracted with 2×50 mL of CH₂Cl₂/iPrOH (5:1). The combined organic phases were dried over sodium sulfate, filtered, and concentrated. The crude material was purified via silica gel chromatography (0-15% acetonitrile/methanol (7:1) in CH₂Cl₂) to deliver the title compound (180 mg, 64% yield over 2 steps) as a light tan solid. ¹H NMR (500 MHz, DMSO-d₆) δ (ppm) 13.1-12.5 (pair of s, 1H, tautomers), 9.46 (s, 1H), 8.30 (s, 1H), 8.26-8.00 (pair of s, 1H, tautomers), 7.90 (s, 1H), 7.50 (m, 1H), 7.41 (m, 1H), 7.32 (m, 1H), 7.02 (app. t, 1H), 4.53 (s, 2H).

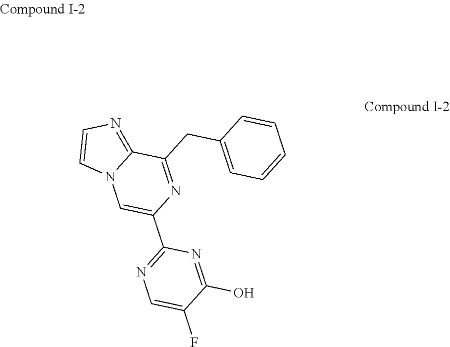 [SMILES: Oc1nc(-c2cn3ccnc3c(Cc3ccccc3)n2)ncc1F]

2-(8-Benzylimidazo[1,2-a]pyrazin-6-yl)-5-fluoropyrimidin-4-ol (Compound I-2) was synthesized according to General Procedure A as a white solid (25 mg, 14% overall yield). The reaction conditions (such as reagents ratio, temperature and reaction time) and purification methods were modified as needed. ¹H NMR (500 MHz, methanol-d₄) δ (ppm) 9.30 (s, 1H), 8.09 (s, 1H), 7.99 (d, 1H), 7.80 (s, 1H), 7.40 (d, 2H), 7.17 (t, 2H), 7.07-7.11 (m, 1H), 4.52 (s, 2H).

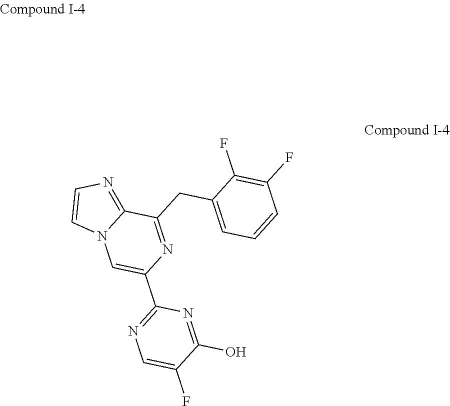 [SMILES: Oc1nc(-c2cn3ccnc3c(Cc3cccc(F)c3F)n2)ncc1F]

2-(8-(2,3-Difluorobenzyl)imidazo[1,2-a]pyrazin-6-yl)-5-fluoropyrimidin-4-ol (Compound 4) was synthesized according to General Procedure A as a light tan solid (150 mg, 67% overall yield). The reaction conditions (such as reagents ratio, temperature and reaction time) and purification methods were modified as needed. ¹H NMR (500 MHz, acetone-d₆) δ (ppm) 10.6 (s, 1H), 9.34 (s, 1H), 8.16 (s, 1H), 7.92 (s, 1H), 7.78 (s, 1H), 7.20 (t, 1H), 7.09 (q, 1H), 7.00 (q, 1H), 4.60 (s, 2H).

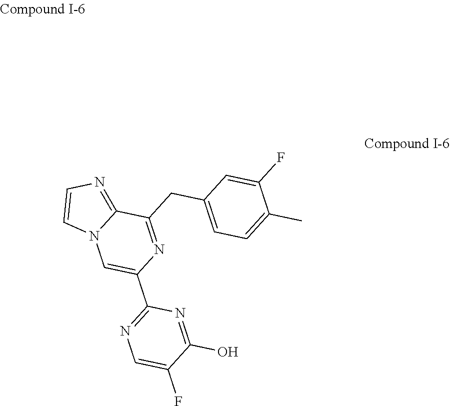 [SMILES: Cc1ccc(Cc2nc(-c3ncc(F)c(O)n3)cn3ccnc23)cc1F]

5-Fluoro-2-(8-(3-fluoro-4-methylbenzyl)imidazo[1,2-a]pyrazin-6-yl)pyrimidin-4-ol (Compound I-6) was synthesized according to General Procedure A as a pale yellow solid (200 mg, 54% overall yield). The reaction conditions (such as reagents ratio, temperature and reaction time) and purification methods were modified as needed. ¹H NMR (500 MHz, DMSO-d₆) δ (ppm) 12.9 (br. s, 1H), 9.44 (s, 1H), 8.29 (s, 1H), 8.16 (br. s, 1H), 7.89 (s, 1H), 7.39 (d, 1H), 7.27 (d, 1H), 7.17 (t, 1H), 4.48 (s, 2H), 2.14 (s, 3H).

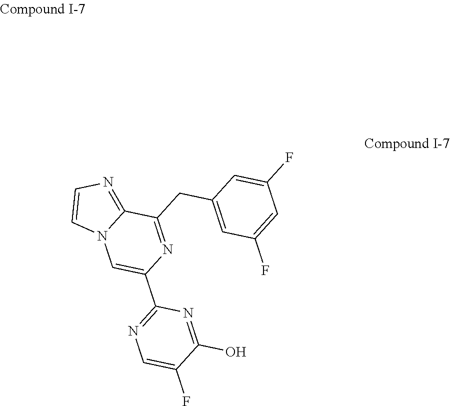 [SMILES: Oc1nc(-c2cn3ccnc3c(Cc3cc(F)cc(F)c3)n2)ncc1F]

2-(8-(3,5-Difluorobenzyl)imidazo[1,2-a]pyrazin-6-yl)-5-fluoropyrimidin-4-ol (Compound I-7) was synthesized according to General Procedure A as a yellow solid (190 mg, 57% overall yield). The reaction conditions (such as reagents ratio, temperature and reaction time) and purification methods were modified as needed. ¹H NMR (500 MHz, DMSO-d₆) δ (ppm) 13.0 (br. s, 1H), 9.47 (s, 1H), 8.31 (s, 1H), 8.22 (br. s, 1H), 7.91 (s, 1H), 7.36 (br. s, 2H), 7.07 (t, 1H), 4.53 (s, 2H).

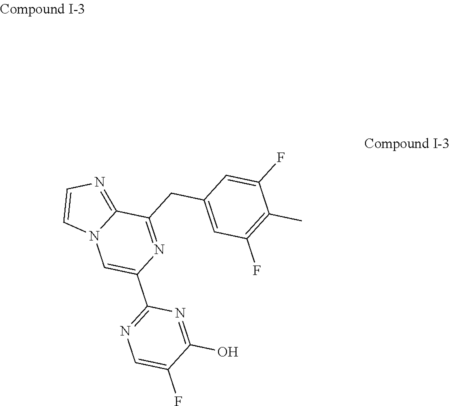 [SMILES: Cc1c(F)cc(Cc2nc(-c3ncc(F)c(O)n3)cn3ccnc23)cc1F]

5-Fluoro-2-(8-(3,5-difluoro-4-methylbenzyl)imidazo[1,2-a]pyrazin-6-yl)pyrimidin-4-ol (Compound I-3) was synthesized according to General Procedure A as a white solid (67 mg, 34% overall yield). The reaction conditions (such as reagents ratio, temperature and reaction time) and purification methods were modified as needed. ¹H NMR (500 MHz, chloroform-d) δ (ppm) 11.1 (br. s, 1H), 9.14 (s, 1H), 8.00-7.91 (m, 2H), 7.87 (s, 1H), 7.00 (d, 2H), 4.56 (s, 2H), 2.14 (s, 3H).

### Compound I-14

The title compound was synthesized in 2 steps:

### Step 1: Synthesis of 8-(2,5-difluoro-4-methylbenzyl)imidazo[1,2-a]pyrazine-6-carboximidamide

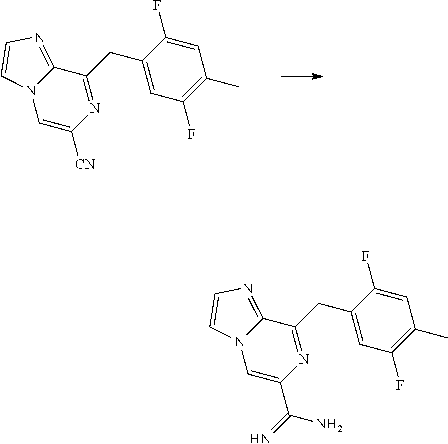 [SMILES: Cc1cc(F)c(Cc2nc(C#N)cn3ccnc23)cc1F ; Cc1cc(F)c(Cc2nc(C(=N)N)cn3ccnc23)cc1F]

To a suspension of 8-(2,5-difluoro-4-methylbenzyl)imidazo[1,2-a]pyrazine-6-carbonitrile (2.5 g, 8.9 mmol, 1.0 equiv.) in methanol (44 mL) was added a solution of 0.50 N sodium methanolate in methanol (18 mL, 8.9 mmol, 1.0 equiv.). After 4 h of stirring at ambient temperature, an additional portion of 0.50 N sodium methanolate in methanol (5.3 mL, 2.7 mmol, 0.3 equiv.) was added and stirring was continued for another 2 h. Ammonium chloride (470 mg, 8.9 mmol, 1.0 equiv.) was then added. After 16 h, the reaction mixture was concentrated in vacuo, suspended in aqueous saturated NaHCO₃ solution, and stirred for 20 min. The solid was collected by filtration and washed with 3 volumes of water and 2 volumes of ether. The crude product was re-suspended in 100 mL of acetonitrile with heating, diluted with ether, and filtered. The filter cake was washed with 3 volumes of ether and dried to afford a tan solid (2.2 g, 83% yield). It was used in the next step without further purification. LC/MS ES⁺ m/z=302.1 [M+H]⁺.

### Step 2: Synthesis of 5-fluoro-2-(8-(2,5-difluoro-4-methylbenzyl)imidazo[1,2-a]pyrazin-6-yl)pyrimidin-4-ol

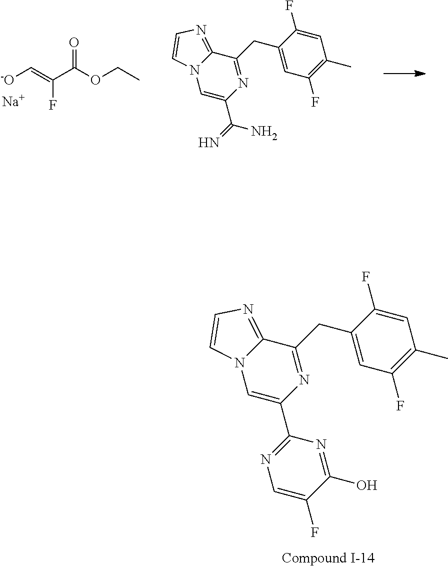

To 8-(2,5-difluoro-4-methylbenzyl)imidazo[1,2-a]pyrazine-6-carboximidamide (1.9 g, 6.2 mmol, 1.0 equiv.) in ethanol (31 mL) was added sodium (Z)-3-ethoxy-2-fluoro-3-oxoprop-1-en-1-olate (2.9 g, 19 mmol, 3.0 equiv.). The solution was heated in a sealed vessel at 90° C. for 18 h. After cooling to ambient temperature, 2.5 N ethanolic HCl solution (7.4 mL, 19 mmol, 3.0 equiv.) was added. The resultant mixture was concentrated in vacuo, suspended in acetonitrile (100 mL) with heating. After slight cooling, ether (100 mL) was added and the mixture was stirred for 10 min. The solid was collected by filtration and washed with 3 volumes of ether. The resultant solid was re-suspended in water, stirred for 1 h and filtered. The crude material was purified via preparative reverse phase HPLC (10-70% acetonitrile/water with 0.1% trifluoroacetic acid as additive). Impure fractions were repurified via preparative reverse phase HPLC (10-50% acetonitrile/water with 0.1% trifluoroacetic acid as additive) to deliver the title compound (840 mg, 37% yield) as an off-white solid. ¹H NMR (500 MHz, methanol-d₄) δ (ppm) 9.43 (s, 1H), 8.21 (s, 1H), 8.08 (br. s, 1H), 7.89 (s, 1H), 7.09 (m, 1H), 7.00 (m, 1H), 4.63 (s, 2H), 2.23 (s, 3H).

### Na+ Salt of Compound I-14

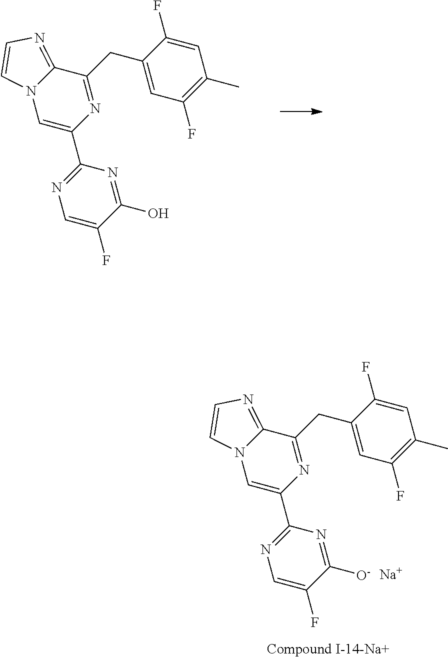 [SMILES: Cc1cc(F)c(Cc2nc(-c3ncc(F)c(O)n3)cn3ccnc23)cc1F ; Cc1cc(F)c(Cc2nc(-c3ncc(F)c([O-])n3)cn3ccnc23)cc1F ; [Na+]]

To an off-white suspension of 5-fluoro-2-(8-(2,5-difluoro-4-methylbenzyl)imidazo[1,2-a]pyrazin-6-yl)pyrimidin-4-ol (Compound I-14, 10 g, 27 mmol) in 450 mL of anhydrous MeOH under a nitrogen atmosphere was added a solution of 0.50 N sodium methanolate in methanol (54 mL, 27 mmol). After brief sonication, the resultant lightyellow solution was stirred at ambient temperature for 15 min and concentrated to dryness in vacuo. The solid was re-suspended in 250 mL of ether with the aid of sonication and concentrated (two times). The resultant solid was re-suspended in 650 mL of ether and stirred at ambient temperature for 3 h. The solid was collected by vacuum filtration and washed with ether (3×100 mL). After drying overnight on the filter, the product salt was dried in a vacuum oven at 45° C. for 4 days to afford sodium 5-fluoro-2-(8-(2,5-difluoro-4-methylbenzyl)imidazo[1,2-a]pyrazin-6-yl)pyrimidin-4-olate (11 g, 99% yield) as a white solid. ¹H NMR (500 MHz, D₂O) δ (ppm) 8.92 (s, 1H), 7.99 (d, 1H), 7.97 (d, 1H), 7.70 (d, 1H), 6.98 (dd, 1H), 6.86 (dd, 1H), 4.48 (s, 2H), 2.14 (s, 3H).

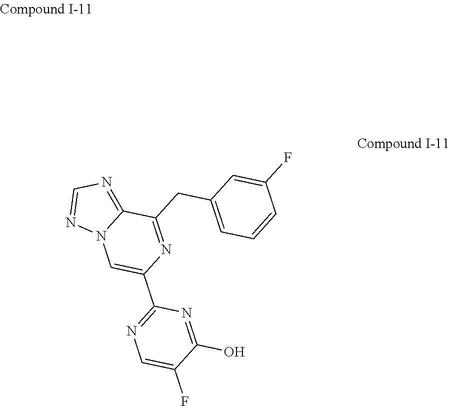 [SMILES: Oc1nc(-c2cn3ncnc3c(Cc3cccc(F)c3)n2)ncc1F]

5-Fluoro-2-(8-(3-fluorobenzyl)-[1,2,4]triazolo[1,5-a]pyrazin-6-yl)pyrimidin-4-ol (Compound I-11) was synthesized according to General Procedure A as a yellow-gold solid (61 mg, 23% overall yield). The reaction conditions (such as reagents ratio, temperature and reaction time) and purification methods were modified as needed. ¹H NMR (500 MHz, DMSO-d₆) δ (ppm) 13.3 (br. s, 1H), 9.60 (s, 1H), 8.86 (s, 1H), 8.24-8.27 (m, 1H), 7.32-7.47 (m, 3H), 7.03-7.06 (m, 1H), 4.59 (s, 2H).

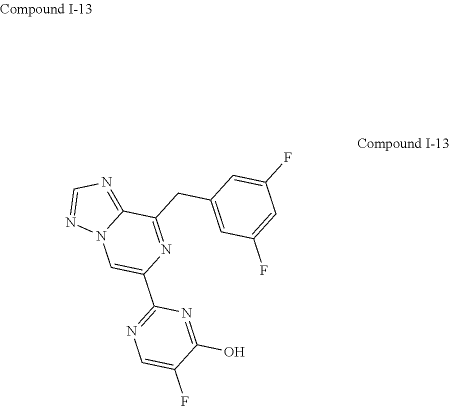 [SMILES: Oc1nc(-c2cn3ncnc3c(Cc3cc(F)cc(F)c3)n2)ncc1F]

2-(8-(3,5-Difluorobenzyl)-[1,2,4]triazolo[1,5-a]pyrazin-6-yl)-5-fluoropyrimidin-4-ol (Compound I-13) was synthesized according to General Procedure A as a brown solid (57 mg, 17% overall yield). The reaction conditions (such as reagents ratio, temperature and reaction time) and purification methods were modified as needed. ¹H NMR (500 MHz, DMSO-d₆) δ (ppm) 13.2 (br. s, 1H), 9.61 (s, 1H), 8.87 (s, 1H), 8.25 (s, 1H), 7.33 (d, 2H), 7.10 (t, 1H), 4.60 (s, 2H).

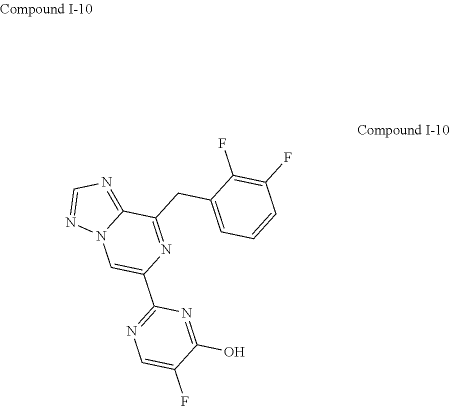 [SMILES: Oc1nc(-c2cn3ncnc3c(Cc3cccc(F)c3F)n2)ncc1F]

2-(8-(2,3-Difluorobenzyl)-[1,2,4]triazolo[1,5-a]pyrazin-6-yl)-5-fluoropyrimidin-4-ol (Compound I-10) was synthesized according to General Procedure A as a pale-yellow solid (85 mg, 16% overall yield). The reaction conditions (such as reagents ratio, temperature and reaction time) and purification methods were modified as needed. ¹H NMR (500 MHz, DMSO-d₆) δ (ppm) 13.0 (br. s, 1H), 9.62 (s, 1H), 8.85 (s, 1H), 8.23 (s, 1H), 7.29-7.37 (m, 2H), 7.09-7.16 (m, 1H), 4.68 (s, 2H).

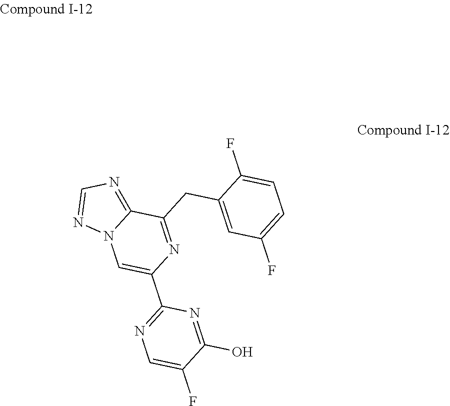 [SMILES: Oc1nc(-c2cn3ncnc3c(Cc3cc(F)ccc3F)n2)ncc1F]

2-(8-(2,5-Difluorobenzyl)-[1,2,4]triazolo[1,5-a]pyrazin-6-yl)-5-fluoropyrimidin-4-ol (Compound I-12) was synthesized according to General Procedure A as an off-white solid (75 mg, 57% yield). The reaction conditions (such as reagents ratio, temperature and reaction time) and purification methods were modified as needed. ¹H NMR (500 MHz, methanol-d₄) δ (ppm) 9.67 (s, 1H), 8.69 (s, 1H), 8.12 (d, 1H), 7.25 (m, 1H), 7.13 (m, 1H), 7.02 (m, 1H), 4.73 (s, 2H).

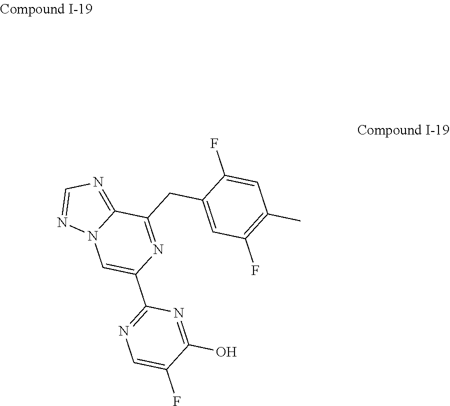 [SMILES: Cc1cc(F)c(Cc2nc(-c3ncc(F)c(O)n3)cn3ncnc23)cc1F]

5-Fluoro-2-(8-(2,5-difluoro-4-methylbenzyl)-[1,2,4]triazolo[1,5-a]pyrazin-6-yl)pyrimidin-4-ol (Compound I-19) was synthesized according to General Procedure A as a tan solid (140 mg, 66% overall yield). The reaction conditions (such as reagents ratio, temperature and reaction time) and purification methods were modified as needed. ¹H NMR (500 MHz, DMSO-d₆) δ (ppm) 13.1 (br. s, 1H), 9.61 (s, 1H), 8.83 (s, 1H), 8.26 (br. s, 1H), 7.35 (br. s, 1H), 7.17 (m, 1H), 4.57 (s, 2H), 2.18 (s, 3H).

### General Procedure B, as Applied to the Synthesis of Compound I-16

The title compound was synthesized in 2 steps:

### Step 1: Synthesis of 8-(2,5-difluorobenzyl)imidazo[1,2-a]pyrazine-6-carboximidamide

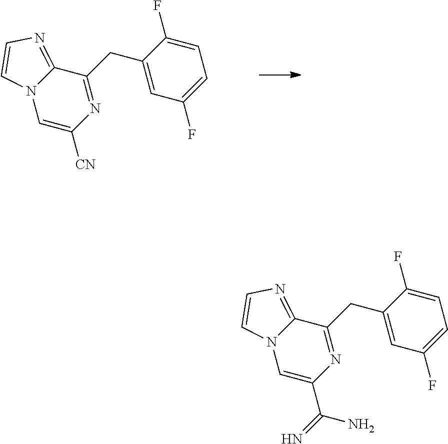 [SMILES: N#Cc1cn2ccnc2c(Cc2cc(F)ccc2F)n1 ; N=C(N)c1cn2ccnc2c(Cc2cc(F)ccc2F)n1]

To a suspension of 8-(2,5-difluorobenzyl)imidazo[1,2-a]pyrazine-6-carbonitrile (490 mg, 1.8 mmol, 1.0 equiv.) in methanol (5.0 mL) was added a solution of 0.50 N sodium methanolate in methanol (3.6 mL, 1.8 mmol, 1.0 equiv.) (note: catalytic or excess amount of sodium methanolate could also be used). After 3 h 45 min of stirring at ambient temperature, ammonium chloride (970 mg, 18 mmol, 10 equiv.) was added and the reaction was stirred for 20 h. The resultant mixture was concentrated in vacuo to a volume of about 2 mL and diluted with EtOAc (20 mL) and 10% aqueous NaHCO₃ solution (10 mL). After stirring for 15 min, the product was collected by filtration, washed with water (10 mL) and dried in vacuo to afford the title compound (420 mg, 80% yield) as an off-white solid. LC/MS ES⁺ m/z=287.9 [M+H]⁺.

### Step 2: Synthesis of 5-chloro-2-(8-(2,5-difluorobenzyl)imidazo[1,2-a]pyrazin-6-yl)pyrimidin-4-ol

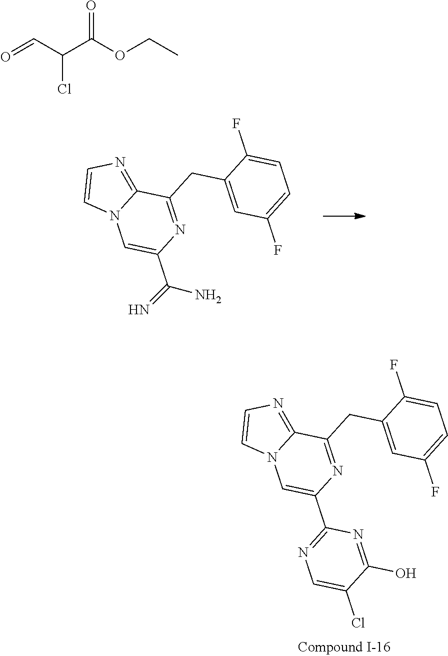

To a suspension of 8-(2,5-difluorobenzyl)imidazo[1,2-a]pyrazine-6-carboximidamide (100 mg, 0.35 mmol) and ethyl 2-chloro-3-oxopropanoate (110 mg, 0.70 mmol) in methanol (1.7 mL) was added a solution of 0.50 N sodium methanolate in methanol (1.4 mL, 0.70 mmol). The reaction was heated in a sealed vial at 65° C. for 2.5h. After cooling to ambient temperature, the resultant mixture was concentrated in vacuo, diluted with water (10 mL), adjusted to pH 3 with 6.0 N aqueous HCl solution, and extracted with 2×15 mL of CH₂Cl₂/iPrOH (8:1). The combined organic phases were dried over sodium sulfate, filtered, and concentrated. The crude material was purified via silica gel chromatography (0-20% acetonitrile/methanol (7:1) in CH₂Cl₂) and repurified via silica gel chromatography (20-100% EtOAc/CH₂Cl₂) to deliver the title compound (37 mg, 28% yield) as an off-white solid. ¹H NMR (500 MHz, DMSO-d₆) δ (ppm) 12.6 (br. s, 1H), 9.54 (s, 1H), 8.36 (br. s, 1H), 8.32 (s, 1H), 7.89 (s, 1H), 7.45 (br. s, 1H), 7.25 (m, 1H), 7.12 (m, 1H), 4.58 (s, 2H).

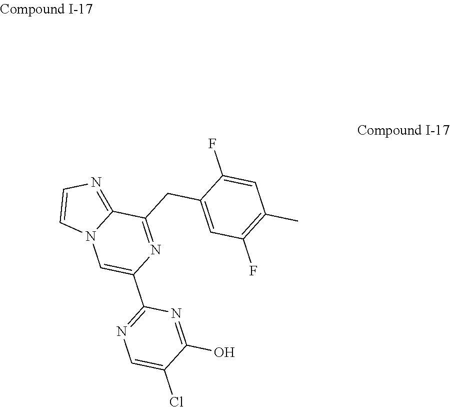 [SMILES: Cc1cc(F)c(Cc2nc(-c3ncc(Cl)c(O)n3)cn3ccnc23)cc1F]

5-Chloro-2-(8-(2,5-difluoro-4-methylbenzyl)imidazo[1,2-a]pyrazin-6-yl)pyrimidin-4-ol (Compound I-17) was synthesized according to General Procedure B as a tan solid (5.2 mg, 2.2% overall yield). The reaction conditions (such as reagents ratio, temperature and reaction time) and purification methods were modified as needed. ¹H NMR (500 MHz, DMSO-d₆) δ (ppm) 9.47 (s, 1H), 8.45 (s, 1H), 8.22 (d, 1H), 7.94 (s, 1H), 7.23 (dd, 1H), 7.16 (dd, 1H), 6.72 (d, 1H), 4.56 (s, 2H), 2.17 (br s, 3H). LC/MS ES⁺ m/z=388.0 [M+H]⁺.

### Compound I-15

The title compound was synthesized in 2 steps:

### Step 1: Synthesis of 8-(2,5-difluoro-4-methylbenzyl)imidazo[1,2-a]pyrazine-6-carboximidamide

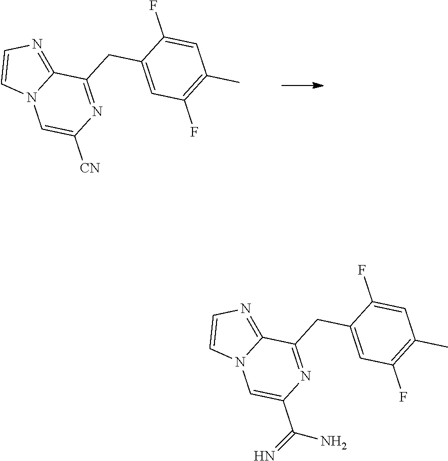 [SMILES: Cc1cc(F)c(Cc2nc(C#N)cn3ccnc23)cc1F ; Cc1cc(F)c(Cc2nc(C(=N)N)cn3ccnc23)cc1F]

8-(2,5-Difluoro-4-methylbenzyl)imidazo[1,2-a]pyrazine-6-carboximidamide was synthesized according to Step 1 of General Procedure A or B as a tan solid (840 mg, 76% yield). The reaction conditions (such as reagents ratio, temperature and reaction time) and purification methods were modified as needed. LC/MS ES⁺ m/z=302.0 [M+H]⁺.

### Step 2: Synthesis of 2-(8-(2,5-difluoro-4-methylbenzyl)imidazo[1,2-a]pyrazin-6-yl)pyrimidin-4-ol

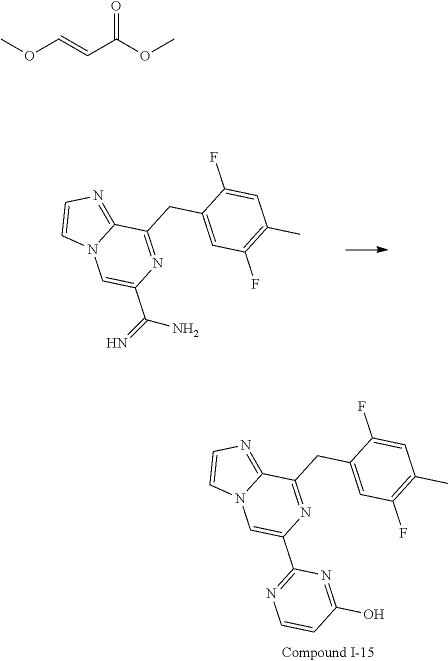

To a suspension of 8-(2,5-difluoro-4-methylbenzyl)imidazo[1,2-a]pyrazine-6-carboximidamide (360 mg, 1.2 mmol) and methyl 3-methoxyacrylate (0.39 mL, 3.6 mmol) in ethanol (6.0 mL) was added Hunig's base (0.63 mL, 3.6 mmol). The reaction was heated in a sealed vial at 90° C. for 3h. After cooling to ambient temperature, the resultant mixture treated with 2.5 N ethanolic HCl solution (1.4 mL, 3.6 mmol) and concentrated to dryness. The crude material was purified via silica gel chromatography (0-20% acetonitrile/methanol (7:1) in CH₂Cl₂) and repurified via silica gel chromatography (0-15% MeOH/CH₂Cl₂) to deliver the title compound (120 mg, 28% yield) as a tan solid. ¹H NMR (500 MHz, DMSO-d₆) δ (ppm) 11.9 (br. s, 1H), 9.52 (s, 1H), 8.31 (s, 1H), 8.07 (br. d, 1H), 7.89 (s, 1H), 7.37 (dd, 1H), 7.16 (dd, 1H), 6.38 (br. d, 1H), 4.54 (s, 2H), 2.18 (s, 3H).

### Compound I-8

The title compound was synthesized in 2 steps:

### Step 1: Synthesis of 8-(2-fluorobenzyl)imidazo[1,2-a]pyrazine-6-carboximidamide

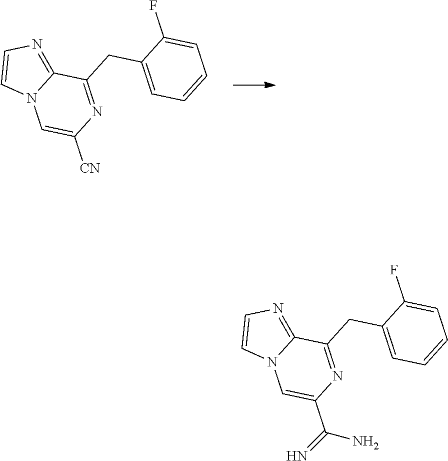 [SMILES: N#Cc1cn2ccnc2c(Cc2ccccc2F)n1 ; N=C(N)c1cn2ccnc2c(Cc2ccccc2F)n1]

8-(2-Fluorobenzyl)imidazo[1,2-a]pyrazine-6-carboximidamide was synthesized according to Step 1 of General Procedure A or B as a cream-colored solid (5.1 g, 91% yield). The reaction conditions (such as reagents ratio, temperature and reaction time) and purification methods were modified as needed. LC/MS ES⁺ m/z=270.2 [M+H]⁺.

### Step 2: Synthesis of 2-(8-(2-fluorobenzyl)imidazo[1,2-a]pyrazin-6-yl)-5-methylpyrimidin-4-ol

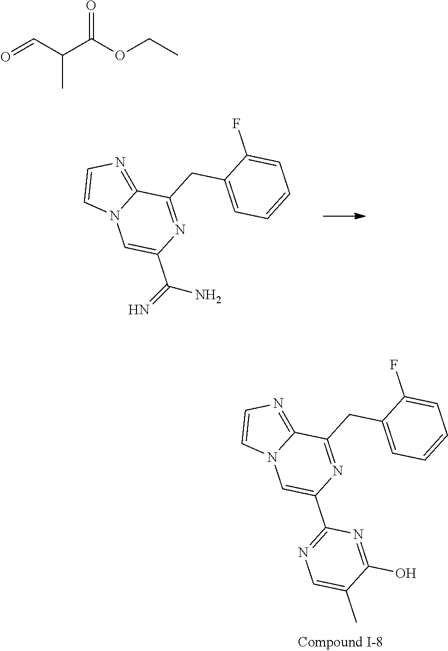

To a solution of 8-(2-fluorobenzyl)imidazo[1,2-a]pyrazine-6-carboximidamide (400 mg, 1.5 mmol) and ethyl 2-methyl-3-oxopropanoate (230 mg, 1.8 mmol) in t-BuOH (9.9 mL) was added potassium hydrogencarbonate (220 mg, 2.2 mmol). The reaction was heated to reflux for 2h. After cooling to ambient temperature, water was added and the product was collected by filtration and dried to deliver the title compound as a cream-colored solid (410 mg, 82% yield). ¹H NMR (500 MHz, DMSO-d₆) δ (ppm) 11.6 (br. s, 1H), 9.48 (s, 1H), 8.30 (s, 1H), 7.94 (br s, 1H), 7.88 (s, 1H), 7.48 (app. t, 1H), 7.29 (m, 1H), 7.19 (m, 1H), 7.11 (app. t, 1H), 4.60 (s, 2H), 1.98 (s, 3H).

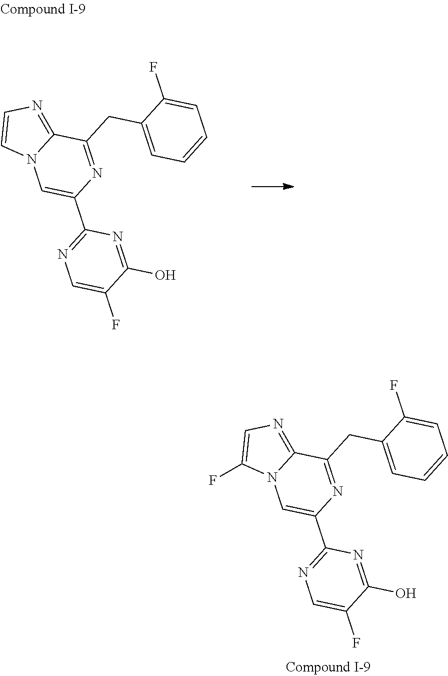 [SMILES: Oc1nc(-c2cn3ccnc3c(Cc3ccccc3F)n2)ncc1F ; Oc1nc(-c2cn3c(F)cnc3c(Cc3ccccc3F)n2)ncc1F]

5-Fluoro-2-(8-(2-fluorobenzyl)imidazo[1,2-a]pyrazin-6-yl)pyrimidin-4-ol (230 mg, 0.67 mmol) in 9.0 mL of acetonitrile-THF (2:1) was treated with sodium hydrogen carbonate (84 mg, 1.0 mmol) and Selectfluor™ (350 mg, 1.0 mmol) and heated at 50° C. Over the course of the experiment, additional portions of sodium hydrogen carbonate (42+28 mg) and Selectfluor™ (180+120 mg) were added. After a total of 49 h, the reaction was cooled to ambient temperature and 20 mL of water was added. The resultant mixture was acidified to pH 3 with 1.0 N aqueous HCl solution and extracted with 2×25 mL of EtOAc. The combined organic phases were dried over sodium sulfate, filtered, and concentrated. The crude material was purified via silica gel chromatography (0-20% acetonitrile/methanol (7:1) in CH₂Cl₂) and repurified via preparative reverse phase HPLC (15-65% acetonitrile/water with 0.1% formic acid as additive) to deliver the title compound (23 mg, 9.7% yield) as a tan solid. ¹H NMR (500 MHz, DMSO-d₆) δ (ppm) 12.6 (br. s, 1H), 8.98 (s, 1H), 8.19 (br. s, 1H), 7.74 (d, 1H), 7.48 (app. t, 1H), 7.28 (m, 1H), 7.19 (m, 1H), 7.10 (app. t, 1H), 4.56 (s, 2H).

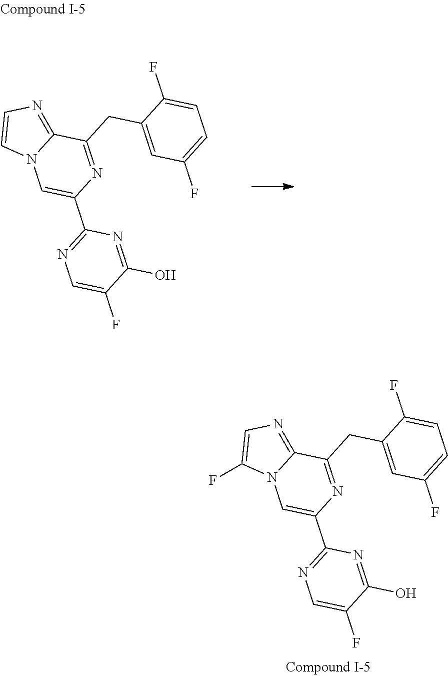 [SMILES: Oc1nc(-c2cn3ccnc3c(Cc3cc(F)ccc3F)n2)ncc1F ; Oc1nc(-c2cn3c(F)cnc3c(Cc3cc(F)ccc3F)n2)ncc1F]

5-Fluoro-2-(8-(2,5-difluorobenzyl)imidazo[1,2-a]pyrazin-6-yl)pyrimidin-4-ol (200 mg, 0.56 mmol) in 10 mL of acetonitrile-THF (1:1) was treated with sodium hydrogen carbonate (94 mg, 1.1 mmol) and Selectfluor™ (400 mg, 1.1 mmol) and heated at 50° C. Over the course of the experiment, additional portions of sodium hydrogen carbonate (3×47 mg) and Selectfluor™ (3×200 mg) were added. After a total of 74 h, the reaction was cooled to ambient temperature and 40 mL of water was added. The resultant mixture was acidified to pH 3 with 1.0 N aqueous HCl solution and extracted with 2×40 mL of CH₂Cl₂/iPrOH (6:1). The combined organic phases were dried over sodium sulfate, filtered, and concentrated. The crude material was purified by silica gel chromatography (0-20% acetonitrile/methanol (7:1) in CH₂Cl₂), preparative reverse phase HPLC (10-70% acetonitrile/water with 0.1% TFA as additive) and a final column chromatography (20-100% EtOAc/hexanes) to deliver the title compound (24 mg, 11% yield) as a white solid.

¹H NMR (500 MHz, DMSO-d₆) δ (ppm) 12.8 (br. s, 1H), 8.99 (s, 1H), 8.20 (br. s, 1H), 7.75 (d, 1H), 7.42 (m, 1H), 7.25 (m, 1H), 7.13 (m, 1H), 4.54 (s, 2H).

### Compound I-18

The title compound was synthesized in 5 steps:

### Step 1: Synthesis of 6,8-dibromo-3-fluoroimidazo[1,2-a]pyrazine

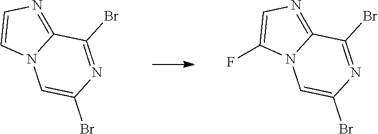 [SMILES: Brc1cn2ccnc2c(Br)n1 ; Fc1cnc2c(Br)nc(Br)cn12]

6,8-Dibromoimidazo[1,2-a]pyrazine (2.4 g, 8.7 mmol) in 40 mL of acetonitrile was treated with Selectfluor™ (4.6 g, 13 mmol) and heated at 50° C. After a 22 h, the reaction was cooled to ambient temperature, poured into 150 mL of half-saturated NaHCO₃ solution and extracted with 2×EtOAc (400 mL total). The combined organic phases were dried over sodium sulfate, filtered, and concentrated. The crude material was purified by silica gel chromatography (0-20% EtOAc/hexanes) to deliver the title compound (580 mg, 23% yield) as an orange solid.

### Step 2: Synthesis of 6-bromo-8-(2,5-difluoro-4-methylbenzyl)-3-fluoroimidazo[1,2-a]pyrazine

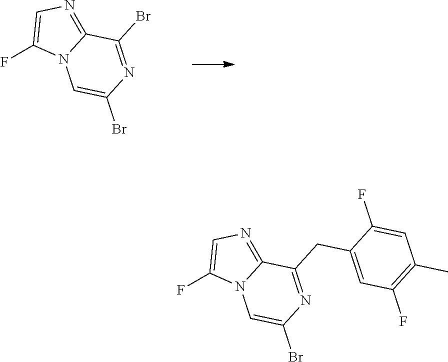 [SMILES: Fc1cnc2c(Br)nc(Br)cn12 ; Cc1cc(F)c(Cc2nc(Br)cn3c(F)cnc23)cc1F]

A suspension of dry zinc powder (240 mg, 3.7 mmol) in THF (3.0 mL) was treated with 1,2-dibromoethane (30 mL, cat.) and the resultant mixture was heated at 50° C. Chlorotrimethylsilane (30 mL, cat.) was then added. After 15 min, the mixture was cooled to ambient temperature. Dry lithium chloride (170 mg, 3.9 mmol) was added, followed by dropwise addition of a solution of 1-(bromomethyl)-2,5-difluoro-3-methylbenzene (480 mg, 2.2 mmol) in THF (2.0 mL) (note: exothermic reaction). The mixture was stirred for 1 h at ambient temperature. Meanwhile, a slurry of 6,8-dibromo-3-fluoroimidazo[1,2-a]pyrazine (580 mg, 2.0 mmol) and Pd(PPh₃)₂Cl₂ (41 mg, 0.059 mmol) in THF (3.0 mL) was degassed with nitrogen. The freshly formed zincate solution was transferred to this slurry via a syringe and rinsed with 2×0.5 mL THF to ensure complete transfer. The resultant mixture was stirred at ambient temperature for 1 h 20 min and then at 40° C. for 4 h. After cooling to ambient temperature, the reaction was quenched with 4 mL of saturated NH₄Cl solution. The organic layer was concentrated, diluted with CH₂Cl₂ (10 mL) and filtered through a bed of Celite. The filtrate was concentrated to yield a brown residue which was purified by silica gel chromatography (compound loaded with CH₂Cl₂ and eluted with 0-10% EtOAc/hexanes) to deliver the title compound (440 mg, 63% yield) as a yellow solid.

### Step 3: Synthesis of 8-(2,5-difluoro-4-methylbenzyl)-3-fluoroimidazo[1,2-a]pyrazine-6-carbonitrile

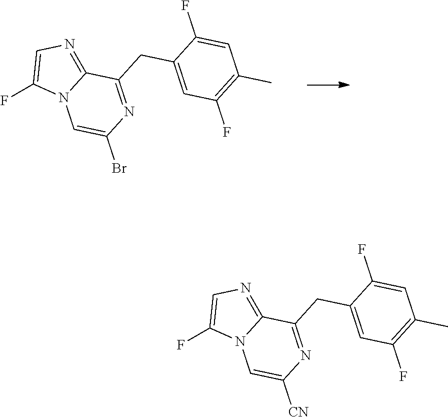 [SMILES: Cc1cc(F)c(Cc2nc(Br)cn3c(F)cnc23)cc1F ; Cc1cc(F)c(Cc2nc(C#N)cn3c(F)cnc23)cc1F]

A reaction mixture comprised of 6-bromo-8-(2,5-difluoro-4-methylbenzyl)-3-fluoroimidazo[1,2-a]pyrazine (440 mmol, 1.2 mmol), zinc cyanide (100 mg, 0.87 mmol), Pd₂(dba)₃ (46 mg, 0.050 mmol) and 1,1′-bis(diphenylphosphino)ferrocene (dppf) (41 mg, 0.075 mmol) in anhydrous DMF (5.0 mL) was degassed with nitrogen and then heated at 90° C. for 6 h. The reaction was cooled to ambient temperature and treated with CH₂Cl₂ (50 mL), water (40 mL) and 28% ammonium hydroxide solution (4.0 mL). The aqueous layer was extracted with CH₂Cl₂ (50 mL). The combined organic layers were dried over Na₂SO₄, filtered and concentrated to afford a brown oil which was purified by column chromatography (0 to 20% EtOAc/hexanes gradient) to afford the title compound as a light tan solid (310 mg, 81% yield). LC/MS ES⁺ m/z=302.8 [M+H]⁺.

### Step 4: Synthesis of 8-(2,5-difluoro-4-methylbenzyl)-3-fluoroimidazo[1,2-a]pyrazine-6-carboximidamide

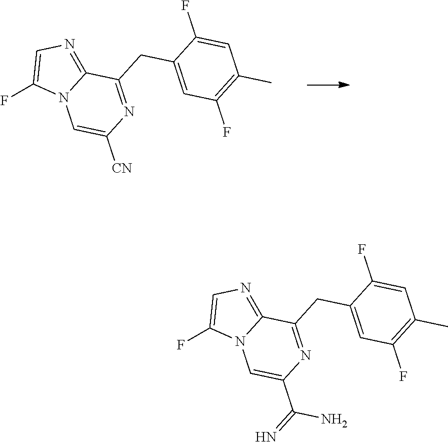 [SMILES: Cc1cc(F)c(Cc2nc(C#N)cn3c(F)cnc23)cc1F ; Cc1cc(F)c(Cc2nc(C(=N)N)cn3c(F)cnc23)cc1F]

To a suspension of 8-(2,5-difluoro-4-methylbenzyl)-3-fluoroimidazo[1,2-a]pyrazine-6-carbonitrile (150 mg, 0.50 mmol) in methanol (6.0 mL) was added a solution of 0.50 N sodium methanolate in methanol (1.0 mL, 0.50 mmol). After 4 h 30 min of stirring at ambient temperature, ammonium chloride (270 mg, 5.0 mmol) was added, and the reaction was stirred for 18 h. The resultant mixture was concentrated in vacuo, treated with 10% aqueous NaHCO₃ solution (10 mL) and sonicated to afford a suspension. After stirring for 1 h, the product was collected by filtration, washed with water (10 mL) and dried in vacuo to afford the title compound (170 mg, >100% yield) as a light tan solid. It was used in the next step without further purification. LC/MS ES⁺ m/z=319.7 [M+H]⁺.

### Step 5: Synthesis of 2-(8-(2,5-difluoro-4-methylbenzyl)-3-fluoroimidazo[1,2-a]pyrazin-6-fluoropyrimidin-4-ol

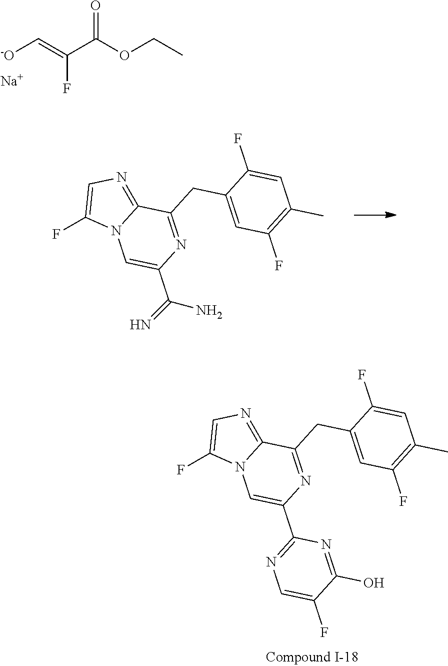

To a suspension of 8-(2,5-difluoro-4-methylbenzyl)-3-fluoroimidazo[1,2-a]pyrazine-6-carboximidamide (0.50 mmol, theoretical amount from previous step) in ethanol (5.0 mL) was added sodium (Z)-3-ethoxy-2-fluoro-3-oxoprop-1-en-1-olate (310 mg, 2.0 mmol). The reaction was heated in a sealed vial at 90° C. for 16h. After cooling to ambient temperature, the mixture was diluted with water (7.5 mL), adjusted to pH 4 with 1N aqueous HCl solution. The resultant tan solid was collected by filtration, washed with water (50 mL) and ethyl ether (30 mL), and dried to afford the title compound (140 mg, 71% yield over 2 steps) as a brown solid. ¹H NMR (500 MHz, DMSO-d₆) δ (ppm) 12.8 (br. s, 1H), 8.98 (s, 1H), 8.22 (br. s, 1H), 7.74 (d, 1H), 7.35 (br. s, 1H), 7.15 (m, 1H), 4.50 (s, 2H), 2.18 (s, 3H).

### Compound I-20

The title compound was synthesized in 2 steps:

### Step 1: Synthesis of 8-(2,5-difluorobenzyl)imidazo[1,2-a]pyrazine-6-carboximidamide

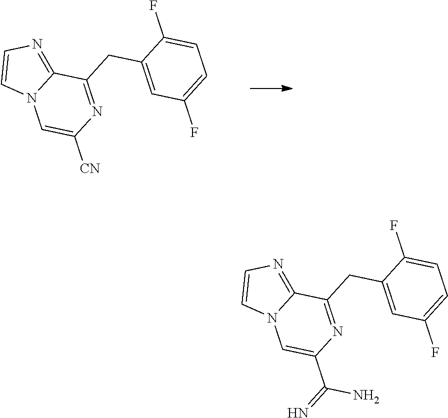 [SMILES: N#Cc1cn2ccnc2c(Cc2cc(F)ccc2F)n1 ; N=C(N)c1cn2ccnc2c(Cc2cc(F)ccc2F)n1]

To a solution of 8-(2,5-difluorobenzyl)imidazo[1,2-a]pyrazine-6-carbonitrile (500 mg, 1.9 mmol) in methanol (22 mL) was added a solution of 25 wt % sodium methanolate in methanol (2.1 mL, 9.3 mmol). After 1 h of stirring at ambient temperature, ammonium chloride (1.0 g, 19 mmol) was added and the reaction was allowed to stir overnight. The reaction mixture was concentrated in vacuo, diluted with half-saturated NaHCO₃ solution (20 mL) and 1.0 N sodium hydroxide solution (2.0 mL), and extracted with 2×20 mL of EtOAc. The combined organic phases were dried over sodium sulfate, filtered, and concentrated to afford the crude product as a brown solid. It was used in the next step without further purification. LC/MS ES⁺ m/z=288.1 [M+H]⁺.

### Step 2: Synthesis of 5-fluoro-2-(8-(2,5-difluorobenzyl)imidazo[1,2-a]pyrazin-6-yl)pyrimidin-4-ol

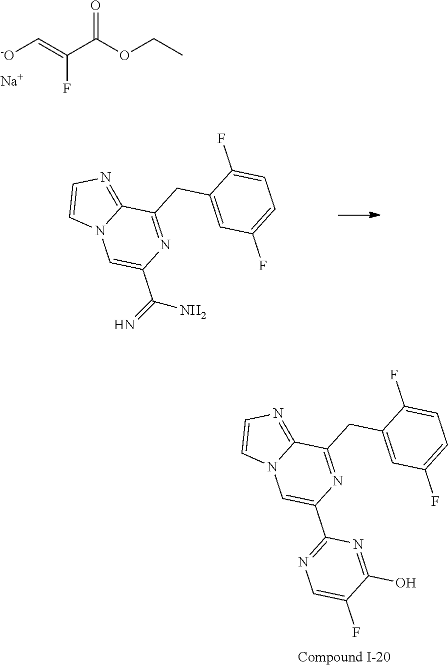

To a suspension of 8-(2,5-difluorobenzyl)imidazo[1,2-a]pyrazine-6-carboximidamide (500 mg, 1.7 mmol) in ethanol (9.0 mL) was added sodium (Z)-3-ethoxy-2-fluoro-3-oxoprop-1-en-1-olate (820 mg, 5.2 mmol). The reaction was heated in a sealed vial at 90° C. for 2h. After cooling to ambient temperature, conc. HCl solution was added dropwise to acidify the mixture to pH 4. The resultant mixture was concentrated in vacuo. Purification by preparative reverse phase HPLC (acetonitrile-water gradient with 0.1% TFA as additive) afforded the title compound (200 mg, 28% yield over 2 steps) as a yellow solid. ¹H NMR (500 MHz, DMSO-d₆) δ (ppm) 12.6 (br. s, 1H), 9.49 (s, 1H), 8.32 (s, 1H), 8.19 (br. s, 1H), 7.89 (s, 1H), 7.43 (s, 1H), 7.25 (m, 1H), 7.13 (m, 1H), 4.58 (s, 2H).

### Na+ Salt of Compound I-20

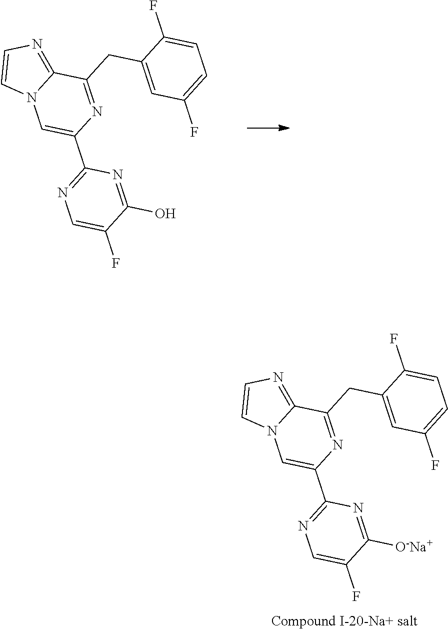

To a light tan suspension of 5-fluoro-2-(8-(2,5-difluorobenzyl)imidazo[1,2-a]pyrazin-6-yl)pyrimidin-4-ol (Compound I-20, 10 g, 28 mmol) in 450 mL of anhydrous MeOH under a nitrogen atmosphere was added a solution of 0.50 N sodium methanolate in methanol (57 mL, 28 mmol). After brief sonication, the resultant light orange solution was stirred at ambient temperature for 15 min and concentrated to dryness in vacuo. The solid was re-suspended in 200 mL of ether with the aid of sonication and concentrated (two times). The resultant solid was re-suspended in 500 mL of ether and stirred at ambient temperature for 3 h. The solid was collected by vacuum filtration and washed with ether (3×100 mL). After drying overnight on the filter, the product salt was dried in a vacuum oven at 45° C. for 5 days to afford sodium 5-fluoro-2-(8-(2,5-difluorobenzyl)imidazo[1,2-a]pyrazin-6-yl)pyrimidin-4-olate (11 g, 99% yield) as a light tan solid.

¹H NMR (500 MHz, D₂O) δ (ppm) 8.90 (s, 1H), 7.98 (d, 1H), 7.95 (d, 1H), 7.70 (d, 1H), 7.10 (m, 1H), 6.98-6.89 (m, 2H), 4.53 (s, 2H).

### Biological Section

### Assessment of the Biological Properties of the Compounds of Formula I

The present invention also provides the assessment of the biological properties of compounds of Formula I. Representatives of the compounds of this invention have been tested in vitro for their activity as sGC stimulators in a variety of cells and assays and in vivo for their ability to reduce blood pressure in animals. Reduction of blood pressure is used as an indication of the ability of the compounds to engage the target peripherally in vivo. Further tests are used as an indication of the ability of these compounds to cross the BBB, to engage the target in the CNS, to increase levels of cGMP in the CNS and to provoke functional responses in animals as a result. These biological properties represent another embodiment of the present invention.

### Example 2: Biological Activity Measurement by the cGMP Glo Sensor Cell-Based Assay, 384-Well Format

Human embryonic kidney cells (HEK293) cells expressing GloSensor™ 40F cGMP (Part No: CS182801, Promega) were used to evaluate the activity of test compounds. The luminescent biosensors (engineered luciferase) that were incorporated into these cells detect cGMP formed by the compounds stimulating the sGC enzyme and emit luminescence.

cGMP GloSensor cells were maintained in Dulbecco's Modification of Eagle's Medium (DMEM) supplemented with fetal bovine serum (FBS, 10% final) and hygromycine (200 ug/ml). The day before assay, cells were plated in DMEM with 10% FBS in a 50 μL volume at a density of 1.5×10⁴ cells/well in a poly-D-lysine coated 384-well flat white-bottom plate (Corning Cat No 35661). Cells were incubated overnight at 37° C. in a humidified chamber with 5% CO₂. The next day, medium was removed and cells were replaced with 40 ul/well of GloSensor™, 2 mM (Promega Cat No E1291). Cells were treated for 90 minutes at 25° C. to allow the substrate to equilibrate in the cells. Test compounds and Diethylenetriamine NONOate (DETA-NONOate) was diluted to 3 mM (20×) in serum-free CO₂ independent medium and serially diluted at 4× dilutions to create 5× dose curve from which 10 ul was added to the wells (x μM concentration for test compound solution and 10 μM concentration for DETA-NONOate solution; wherein x is one of the following final concentrations: 30 μM, 7.5 μM, 1.9 μM, 469 nM, 117 nM, 29.3 nM, 7.3 nM, 1.83 nM, 0.46 nM, 0.11 nM, 0.03 nM) For the kinetics studies, luminescense was measured right away for 0.2 sec per well with Envision (Perkin Elmer). For endpoint SAR screening, data were collected after 55 min incubation at room temperature.

Concentration response data were analyzed using a 4-parameter fit (log (agonist) vs. response-variable slope). The EC₅₀ was interpolated from the curve fit and is defined as the concentration at which the compound elicits 50% of its maximal response. When the experiment was carried out multiple times for a given compound, the geometrical mean of all experiments is reported.

Table A below summarizes the values of EC₅₀ in the Glo assay for compounds of the invention.

TABLE A

| Compound | Glo | Compound | Glo |
|---|---|---|---|
| No. | EC₅₀ (nM) | No. | EC₅₀ (nM) |
| I-21 | B | I-5 | A |
| I-2 | B | I-14* | A |
| I-1 | B | I-16 | A |
| I-4 | B | I-15 | B |
| I-23 | B | I-19 | B |
| I-20* | A | I-17 | A |
| I-11 | B | I-18 | A |
| I-13 | B | I-12 | B |
| I-10 | B | I-3 | A |
| I-6 | A | I-8 | C |
| I-7 | B | I-9 | B |

sGC enzyme activity values in HEK cells, determined by the GloSensor assay. Code definitions for the sGC enzyme activity values, expressed as EC₅₀ which is defined as the concentration at which the compound elicits 50% of its maximal response: EC₅₀ ≤ 100 nM = A; 100 nM < EC₅₀ ≤ 1000 nM = B; 1000 nM < EC₅₀ = C.
*For Compounds I-20 and I-14, both the free acid and the sodium salt were used and the results here are the average of all experiments run independently of form.

### Example 3. Biological Activity Measurement by the cGMP Neuronal Cell-Based Assay

Rat primary neurons were isolated from fetuses of 18-day pregnant Sprague-Dawley females. The fetuses were collected in Hanks' balanced salt solution (HBSS) and brains were rapidly removed. The cerebral hippocampi were isolated and mechanically fragmented. Further tissue digestion was performed with 0.25% (wt/vol) trypsin solution in HBSS without Ca2+ and Mg2+ for 15 min at 37° C. After trypsination, cells were washed and resuspended in neurobasal medium supplemented with 0.5 mM L-glutamine, 12.5 uM glutamic acid, 2% B-27 and 100 U/mL penicillin, and 100 μg/mL streptomycin. Cells were plated at a density of 26×10³ or 3×10⁴ or 4×10⁴ cells/well in a poly-D-lysine coated 384-well flat clear-bottom plate (Corning Cat No 354662). Cells were incubated 6-7 days at 37° C. in a humidified chamber with 5% CO₂. Media was removed and cells were washed 1× with HBSS containing Ca2+ and Mg2+, and replaced with 40 uL HBSS containing 0.5 mM IBMX, and incubated for 15 minutes at 37° C. 10 uL of a 5× stock of test compounds with diethylenetriamine NONOate (DETA-NO) was added. Final concentration of DETA-NO was either 10 μM or 30 μM. Cells were incubated for 20 min at 37° C. Medium was removed, 50 uL of ice-cold 10% acetic acid was added, and incubated for 60 minutes at 4° C. Following centrifugation at 4° C. for 5 minutes at 1000×g to pellet cell debris, the supernatant was aspirated to a clean plate and the samples were analyzed for cGMP content. cGMP concentrations were determined from each sample using LC-MS/MS.

Concentration response data were analyzed using a 4-parameter fit (log (agonist) vs. response-variable slope). The EC₅₀ was interpolated from the curve fit and is defined as the concentration at which the compound elicits 50% of its maximal response. When the experiment was carried out multiple times for a given compound, the geometrical mean of all experiments is reported.

Table B below summarizes the values of EC₅₀ in the neuronal assay for compounds of the invention.

TABLE B

| Compound | sGC Neuron | Compound | sGC Neuron |
|---|---|---|---|
| No. | EC₅₀ (nM) | No. | EC₅₀ (nM) |
| I-21 | A | I-3 | A |
| I-2 | B | I-15 | B |
| I-1 | B | I-9 | A |
| I-4 | A | I-19 | A |
| I-23 | A | I-5 | A |
| I-20* | A | I-14* | A |
| I-16 | A | I-12 | A |
| I-6 | A | I-7 | A |

Neuronal-based cell assay. EC₅₀ ≤ 100 nM = A; 100 nM < EC₅₀ ≤ 1000 nM = B; 1000 nM < EC₅₀ = C.
*For Compounds I-20 and I-14, both the free acid and the sodium salt were used and the results here are the average of all experiments run independently of form.

### Example 4: Biological Activity Measurement on Human α2β1 sGC Isozyme Stably Expressed in CHO-K1 Cells

sGC stimulators were dissolved in DMSO as a 10 mM solution and stored at −20° C. To achieve desired test concentrations, stock concentrations were serially diluted into DMSO and then diluted to the appropriate concentration in assay buffer.

CHO-K1 cells stably transfected with human α2β1 sGC isozyme (generated by GenScript for Ironwood) were cultured in F-12K medium (ATCC catalog #30-2004) with 10% fetal calf serum, 4 μg/mL puromycin (Gibco catalog #A11138-03) and 0.4 mg/mL of geneticin (Gibco catalog #10131-027) in a 95% humidified atmosphere containing 5% CO₂ in air at 37° C. For GC activity assays, cells were seeded in 384 well poly-D-lysine coated flat bottom plates (Fisher Scientific #08-774-311) in either 50 μL or 70 μL medium at a density of either 3×10⁴ cells/well or 15×10³, respectively. Cells were incubated for 24 hours at 37° C. in a humidified chamber supplemented with 5% CO₂.

For each test concentration, the compound was diluted in 100% DMSO to 100-fold of its final assay concentration. Immediately prior to the assay, the solution was diluted 20-fold into HBSS containing calcium, magnesium, and 50 μM DETA-NONOate (5× the final assay concentration). Medium was removed and cells were washed once with 40 μL of HBSS. Cells were then incubated with 40 μL of a solution containing 0.5 mM IBMX in HBSS for 15 min at 37° C. 10 μL from the sGC stimulator/HBSS/DETA-NONOate plate was added to the cells, which were incubated for an additional 20 min at 37° C. Final DMSO concentration was 1%, final DETA-NONOate concentration was 10 μM; and final compound concentrations were 30,000 nM, 6000 nM, 1200 nM, 240 nM, 48 nM, 9.6 nM, 1.92 nM, 0.384 nM, 0.077 nM, 0.015 nM, or 0.003 nM.

Following incubation with compound, assay buffer was removed and 50 μL of ice-cold 10% acetic acid+150 ng/mL internal standard (+3 cGMP) was added to each well. Samples were incubated on ice for 30-60 min. Following centrifugation at 4° C. for 5 min at 1000×g to pellet cell debris, the supernatant was transferred to a clean plate and the samples analyzed for cGMP content.

Data were analyzed with a 4-parameter fit (log(agonist) vs. response-variable slope) using GraphPad Prism Software v.8. The EC₅₀ was interpolated from the curve fit and is defined as the concentration at which the compound elicits 50% of its maximal response. When the experiment was carried out multiple times for a given compound, the geometrical mean of all experiments is reported.

Table C below summarizes the values of EC₅₀ in the CHO assay for compounds of the invention.

TABLE C

| Compound | sGC CHO | Compound | sGC CHO |
|---|---|---|---|
| No. | EC₅₀ (nM) | No. | EC₅₀ (nM) |
| I-21 | A | I-15 | B |
| I-2 | B | I-19 | A |
| I-17 | A | I-18 | A |
| I-4 | A | I-5 | A |
| I-23 | B | I-14* | A |
| I-20* | A | I-16 | A |

CHO cell assay. EC₅₀ ≤ 100 nM = A; 100 nM < EC₅₀ ≤ 1000 nM = B; 1000 nM < EC₅₀ = C.
*For Compounds I-20 and I-14, both the free acid and the sodium salt were used and the results here are the average of all experiments run independently of form.

### Example 5: Blood Pressure Effects in Normotensive Rats, Following Acute Dose at Multiple Concentrations for Representative Compounds of the Invention

### a) Compound I-14

Male, normotensive Sprague Dawley rats were purchased from Charles River Laboratories. These rats have indwelling femoral artery catheters installed. Animals were harnessed to a tether system and attached to pressure transducers to monitor cardiovascular (CV) parameters, specifically mean arterial pressure (MAP) and heart rate (HR). Animals were acclimated to the system overnight and baseline CV parameters collected. The conscious, freely moving rats were then administered a single oral dose of the Compound I-14 (doses generated from the sodium salt of Compound I-14) in Milli-Q water at 1, 3, 10 and 30 mg/kg. A blood sample was collected from each animal through the catheter line at pre-dose and 2 hours post-dose for compound concentration quantification. Hemodynamic measures were recorded for ten hours post-dose. Fifty-four male rats were used for these studies and they were ordered to be received at a body weight range of 250-275 grams. They were single housed under controlled conditions of temperature (21±1° C.), relative humidity (36±1%) and placed in a 12-hr light-dark cycle (lights on at 6:00 AM and off at 6:00 PM) room at SmartLabs vivarium (21 Erie Street, Cambridge, MA) under protocol MIL-110. Animals were allowed ad libitum access to chow (LabDiet Prolab Isopro RMH 3000, St. Louis, MO) and water. Two sets of studies were carried out. For the first set of studies Compound I-14 was formulated at 0.3 and 1.0 mg/ml in Milli-Q water and frozen at −20° C. For the second set of studies, the sodium salt of Compound I-14 was weighed at Cyclerion Therapeutics and reconstituted at SmartLabs to provide 0.1, 0.3, 1.0 and 3.0 mg/ml solutions of Compound I-14 in Milli-Q water. Formulations prepared were thawed less than 4 hours before dosing and stored at room temperature or were made less than 2 hours before dosing and stored at room temperature.

Studies were conducted over 6 separate sessions. The total number of subjects and treatment assignments are listed in the table below. Animals were initially used within 3 days of receipt and if catheters remained patent, reused once after 6-7 days of washout. No animals were used more than twice.

| | Number | | | |
|---|---|---|---|---|
| | of | | Dose | Dose Con- |
| Group | animals | Treatment | Volume | centration |
| 1 | 13 | Vehicle | 10 mL/kg | — |
| 2 | 9 | 1 mg/kg Compound I-14 | 10 mL/kg | 0.1 mg/mL |
| 3 | 6 | 3 mg/kg Compound I-14 | 10 mL/kg | 0.3 mg/mL |
| 4 | 11 | 10 mg/kg Compound I-14 | 10 mL/kg | 1.0 mg/mL |
| 5 | 6 | 30 mg/kg Compound I-14 | 10 mL/kg | 3.0 mg/mL |

### Measurement of Blood Pressure

This study utilized ADInstruments LabChart (v8) to collect hemodynamic data from conscious, freely moving rats tethered to a blood pressure transducer (Harvard Apparatus cat #APT300). Following an overnight acclimation to the tether and pressure transducer, animals were dosed following a 1-hr baseline recording period. Animals were administered a single oral (P.O.) dose of the sodium salt form of Compound I-14 or vehicle at a dose volume of 10 mL/kg. Data collection was continued for 10 hours post-dose.

ADInstruments LabChart (v8) was used to monitor and export hemodynamic data. Blood pressure and heart rate were continuously monitored, and data were collected at 1000 data points per second then averaged into 10-minute bins for analysis. Change from baseline MAP (ΔBMAP) and HR (ΔBHR) were calculated using the pre-dose baseline averaged over the 1-hr period prior to dosing using Microsoft Excel for Microsoft 365. This 10-minute bin dataset was used to determine peak ΔVMAP, time peak ΔVMAP, peak ΔVHR and time of peak ΔVHR. The dataset was further consolidated into 1-hr bins for the MAP and HR figures and the analysis of ΔBMAP, ΔBMAP, and ΔBHR. The definitions of these terms/abbreviations are summarized below:

| | |
|---|---|
| Δ_{B}MAP, dMAP | Change from baseline mean arterial pressure |
| Δ_{B}HR, dHR | Change from baseline heart rate |
| Δ_{V}MAP | Vehicle-adjusted mean arterial pressure |
| Δ_{V}MAP | Vehicle-adjusted heart rate |
| AOC | Area Over the Curve |

Statistical analyses were performed in Graphpad Prism (v8). Significance, as compared to vehicle-treated rats, for ΔBMAP and ΔBHR data was determined by 2-way repeated measures ANOVA followed by a Dunnett's multiple comparisons test, if there was a missing data point, a mixed-effect analysis was utilized. Vehicle-adjusted MAP (ΔVMAP) was calculated by subtracting ΔBMAP of the vehicle group from the ΔBMAP of each dose group at each timepoint. Vehicle-adjusted HR (ΔVHR) was calculated in a similar manner to ΔVMAP.

Significance of AOC data were determined by one-way ANOVA followed by a Dunnett's multiple comparisons test as compared to vehicle.

Some study data were removed prior to analysis. Data collected 130- and 140-min post-dose were removed due to the 2-hour blood sample collection. A few timepoints for one rat at 1 mg/kg were excluded due to signal loss during the experiment starting at 470. minutes and continuing until the end of the study (600 min). The entire time courses of 5 animals were excluded from all datasets for a variety of reason, including them being outliers for particular analyses or due to signal loss leading to abnormal results.

### Blood Pressure Changes

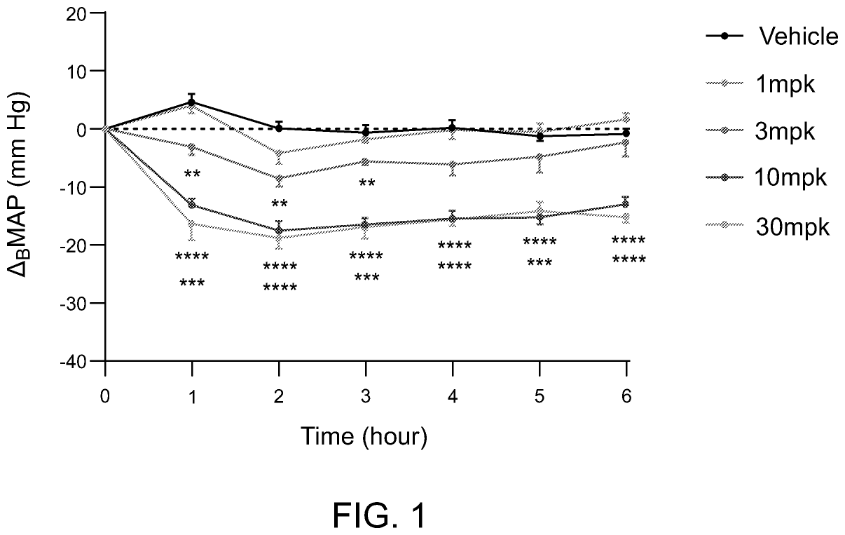

The change from baseline MAP (ΔBMAP) is shown graphically in FIG. 1. There was a greater decrease in MAP (as assessed by change from baseline MAP, ΔBMAP) in rats treated with Compound I-14 than in vehicle-treated rats. The ΔBMAP dataset was significant by 2-way ANOVA (p<0.0001 for treatment and time and p=0.045 for treatment×time interaction). Dunnett's multiple comparison test of the main treatment effect yielded p=ns for 1 mg/kg, p=0.0034 for 3 mg/kg, and p<0.0001 for the 10 and 30 mg/kg doses as compared to vehicle treated rats. A separate Dunnett's multiple comparison test of simple effects at each timepoint and each dose versus vehicle-treated rats show a greater decrease in ΔBMAP throughout the 6-hour post-dose period in rats treated with 10 and 30 mg/kg Compound I-14; at 1-, 2-, and 3-hours post-dose in rats treated with 3 mg/kg Compound I-14; but not in rats treated with 1 mg/kg Compound I-14.

Maximum effect of Compound I-14 on ΔVMAP was calculated by using the 10-minute binned dataset and is shown in Table D below:

TABLE D
Maximum Effect of Compound I-14
on Vehicle-adjusted MAP (ΔVMAP)

| Compound I-14 | Peak Δ_{V}MAP (mm Hg) | Time at Peak (min) |
|---|---|---|
| 1 mg/kg | −6.2 | 90 |
| 3 mg/kg | −10.1 | 70 |
| 10 mg/kg | −22.3 | 30 |
| 30 mg/kg | −24.4 | 30 |

The no effect dose on ΔBMAP is 1 mg/kg as assessed by main effect analysis, simple effects analysis, and by AOC.

### Conclusion

Compound I-14 lowered MAP from baseline and as adjusted from vehicle at 3, 10, and 30 mg/kg.

### b) Compound I-20

Analogous studies to the ones described above were carried out with Compound I-20. The conscious, freely moving rats were administered a single oral dose of Compound I-20 (dosages prepared from the sodium salt of Compound I-20) in Milli-Q water at 1, 3, 10 and 30 mg/kg. A blood sample was collected from each animal through the catheter line at pre-dose and 2 hours post-dose for compound concentration quantification. Hemodynamic measures were recorded for ten hours post-dose.

### Blood Pressure Changes

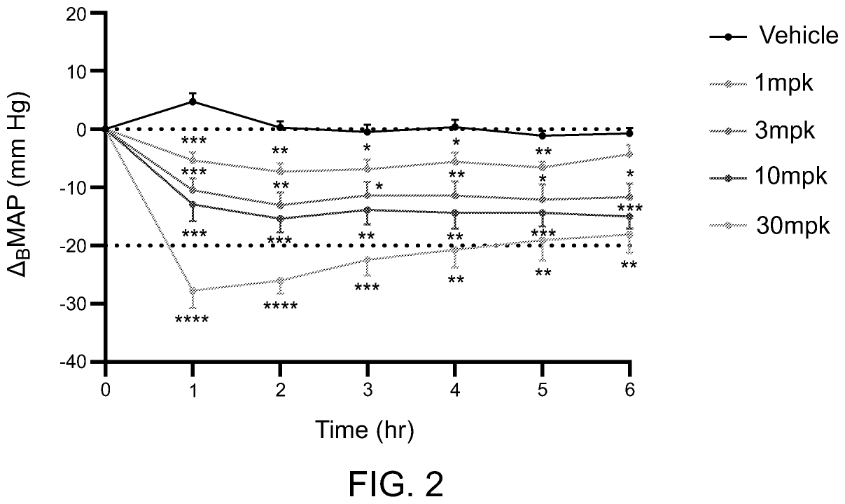

The change from baseline MAP (Δ_{B}MAP) is shown graphically in FIG. 2. There was a greater decrease in MAP (as assessed by change from baseline MAP, Δ_{B}MAP) in rats treated with Compound I-20 than in vehicle-treated rats. The Δ_{B}MAP dataset was significant by 2-way ANOVA (p<0.0001 for treatment and treatment×time interaction; p=0.022 for time). Dunnett's multiple comparison test of the main treatment effect yielded p=0.055 (ns) for 1 mg/kg, p=0.0001 for 3 mg/kg, and p<0.0001 for the 10 and 30 mg/kg doses as compared to vehicle-treated rats. A separate Dunnett's multiple comparison test of simple effects at each timepoint and each dose versus vehicle-treated rats show a greater decrease in MAP throughout the 6-hour post-dose period in rats treated with 3, 10, and 30 mg/kg Compound I-20; at 1-, 2-, 3-, 4-, and 5-hours post-dose in rats treated with 1 mg/kg Compound I-20.

The maximum effect of Compound I-20 on Δ_{V}MAP (vehicle-adjusted MAP) was calculated by using the 10-minute binned dataset and is shown Table E below.

TABLE E
Maximum Effect of Compound I-20
on vehicle-adjusted MAP (Δ_{V}MAP)

| Compound I-20 | Peak Δ_{V}MAP (mm Hg) | Time at Peak (min) |
|---|---|---|
| 1 mg/kg | −14.3 | 20 |
| 3 mg/kg | −19.2 | 20 |
| 10 mg/kg | −20.2 | 30 |
| 30 mg/kg | −38.1 | 30 |

### Conclusions

Compound I-20 lowered MAP at 1, 3, 10, and 30 mg/kg, both from baseline and as adjusted from vehicle.

### c) Other BP Measurements

In similar studies to those described above, Compound I-4, formulated in PEG400 and dosed at 10 mg/kg displayed a maximal reduction of MAP from baseline (Δ_{B}MAP) of 20 mm Hg at 50 min after dosing. In similar studies to those described above, Compound I-20, formulated in PEG400 and dosed at 10 mg/kg displayed a peak Δ_{B}MAP of −26 mm Hg at 42 min after dosing. In another study in which Compound I-20 was tested at 1, 3 or 10 mg/kg and formulated in methylcellulose, the compound was able to reduce MAP from baseline at all doses tested.

### Example 6: sGC Stimulator Induced Phosphorylation of CREB in Rat Primary Neurons

### Objective

To assess the ability of compounds of the invention to activate cAMP response element-binding protein (CREB) in rat primary neurons. CREB is a cellular transcription factor. It binds to DNA sequences called cAMP response elements (CRE), and regulates transcription of the downstream genes (See Bourtchuladze R, et al., Cell 1994; 79 (1): 59-68). CREB has a well-documented role in neuronal plasticity and long-term memory formation in the brain and has been shown to be integral in the formation of spatial memory (See Silva A J, et al., Annual Review of Neuroscience 1998; 21: 127-148). CREB proteins are activated by phosphorylation of Serine 133 by various kinases, including cAMP-dependent protein kinase or Protein Kinase A (PKA), cGMP-dependent protein kinase or Protein Kinase G (PKG), and Ca2+/calmodulin-dependent protein kinases. (See Shaywitz A J and Greenberg M E, Annual Review of Biochemistry 1999; 68 (1): 821-861 and Wong J C, et al., J Cell Biochem 2012: 113(11):3587-98). Stimulation of CREB could have therapeutic benefits for diseases in which cognition, neuronal plasticity, and or neuronal function is impaired.

### Rat Primary Neurons Culture

Neurons were isolated from Sprague Dawley rat embryos on embryonic day 18 (E18). Approximately 10 embryos were obtained from each rat, and whole brains were isolated from the embryos. Hippocampus and cortex were dissected from the brains under a stereoscopic microscope using two fine tweezers. The meninges were carefully removed. After dissection, the tissues were chopped and washed gently once with 10 mL of Ca²⁺ and Mg²⁺ free Hank's balanced salt solution (HBSS, Corning cat #21-022-CM) in a 15-mL conical tube. After washing, 5 mL of a solution of 0.25% trypsin (Invitrogen cat #15090-046) and 0.1% deoxyribonuclease I (DNase I, Sigma cat #DN-25) were added to the tissues, then incubated at 37° C. for 15 min. Next, tissues were washed 3 times with ice-cold HBSS, 3 mL of a solution of 0.1% of DNase I was added, then tissues were slowly pipetted 12 times using a glass Pasteur pipette, followed by centrifugation at 500×g for 10 min. The cell pellet was resuspended in culture medium (Neurobasal medium, Gibco cat #21103-049), 2% of B27 supplement (Gibco Cat #17504-044), 0.5 mM L-glutamine (Corning cat #25-005-C1), 25 μM L-glutamic acid (Sigma cat #G1251) and 1% penicillin/streptomycin (Gibco cat #15070-063). Subsequently, the cell suspension was plated into poly-L-lysine coated 96-well plates at 100,000 cells/well. Twenty-four h after plating, half of the culture medium was removed and replaced with culture medium described above but without glutamic acid. Cells were maintained in a 37° C. humidified incubator with 5% CO₂ and used for assays between days 6-10 post-harvest.

### Assay Conditions

For each test concentration, the compound was diluted in 100% DMSO to 100× its final assay concentration. Immediately prior to the assay, the compound was diluted 1/10 into HBSS (containing calcium and magnesium) (10× the final assay concentration) containing 100 μM DETA-NONOate (10× the final assay concentration). Medium was removed, and cells were washed once with 90 μL HBSS (Corning cat #21-023-CV). Cells were then incubated with 90 μL HBSS for 30 min at 37° C. 10 μL from the test article/HBSS/DETA-NONOate plate was added to the cells and incubated for additional 30 min at 37° C. Final DMSO concentrations were 1%, final DETA-NONOate concentration was 10 μM; and final compound concentrations were 10 μM, 1 μM, 0.1 μM, 0.01 μM, 0.001 μM, 0.0001 μM, 0.00001 μM, and 0.0 μM. Medium was removed, cells were lysed and levels of pCREB was determined according to Cisbio protocols (phospho-CREB (Ser133) catalog #64CREPEG). Plates were read using Envision instrument (PerkinElmer).

### Data Analysis

pCREB was determined for each well and analyzed with a 3-parameter fit (log(agonist) vs. response-variable slope) using GraphPad Prism Software v.8. The EC₅₀ was interpolated from the curve fit and is defined as the concentration at which the sGC stimulator compound elicits 50% of its maximal response. The experiments were carried out multiple times for a given compound and the geometrical mean of all experiments is reported. For both Compounds 14 and 20 the free acid was used in these experiments.

Table F below summarizes the values of EC₅₀ in the pCREB assay for compounds of the invention.

TABLE F

| Compound No. | pCREB EC₅₀ (nM) |
|---|---|
| I-14 | 21 |
| I-20 | 13 |

### Example 7: Rat Cerebrospinal Fluid (CSF) Pharmacokinetic Properties

### Protocol

PK in rats was determined following oral dosing. For the oral (PO) experiments, a group of 6 male Sprague-Dawley rats with an indwelling catheter placed in the cisterna magna were used. The PO group was dosed with 3.0, or 10 mg/kg of a compound formulated as a solution in PEG400 or suspension in 0.5% Tween 80 and 0.5% Methylcellulose in water. PO doses were administered by oral gavage and delivered to the stomach using a syringe and gavage tube. Following oral dosage administration, the gavage tube was flushed with approximately 0.5 mL of water to ensure complete delivery of the full dose.

Plasma samples were collected as follows: samples of CSF and blood were collected at 1 hour, 2, and optionally at 4 hours post-dosing. CSF samples (0.05 mL) were collected through the intracisternal catheter. Blood samples (0.25 mL) were collected through tail nick sampling. These samples were kept on ice until processed for plasma. Blood samples were centrifuged at 3200 rpm for 5 minutes at approximately 5° C. within 1 hour of collection. Plasma was directly transferred to individual Eppendorf tubes (0.125 mL). Plug caps were placed on the tubes and the tubes frozen at approximately −70° C. and stored until analysis. Plasma and CSF was collected and analyzed for the presence of a compound.

### Quantitation of Compounds.

The compound in question and the internal standard were extracted from plasma by precipitation and from CSF by either precipitation or dilution. Samples were analyzed using liquid chromatography (LC) with tandem mass spectrometric detection (MS/MS) using electrospray ionization. The standard curve ranged from 0.1 to 1000 ng/mL. Results of the compounds described herein in this assay are illustrated in Table G below (for a 10 mg/kg dose and/or 3 mg/kg of the compound). Compound concentrations from several animals were combined to give the geometrical mean at each specific dosage and timepoint.

Kp,uu is defined as the concentration ratio of unbound drug in CSF to unbound drug in plasma. Unbound drug in plasma (or free plasma concentration) is calculated by multiplying the total plasma concentration by the unbound fraction as determined by plasma protein binding. The CSF concentration is then divided by the free plasma concentration to determine the Kp,uu. (See e.g., Di et al., J. Med. Chem., 56, 2-12 (2013))

TABLE G

| Compound | Dose | Kp, uu | Kp, uu | Kp, uu |
|---|---|---|---|---|
| No. | (mg/kg) | at 1 h | at 2 h | at 4 h |
| I-20 | 10 | B | C | C |
| I-1 | 3 | A | B | A |
| I-4 | 3 | A | B | B |
| I-20 | 3 | C | C | C |
| I-5 | 3 | C | C | C |
| I-14 | 3 | D | D | D |
| I-16 | 3 | D | D | D |
| I-14 | 10 | D | D | C |
| I-18 | 3 | B | B | B |

Rat CSF PK. Kp, uu ≤ 1 = A; 1 < Kp, uu ≤ 2 = B; 2 < Kp, uu ≤ 3 = C, 3 < Kp, uu = D.

### Example 8: Microdialysis Experiments in Rat Brain

### Objective

The goal of the current study was to evaluate levels of sGC stimulators of the invention in the hippocampus and striatum interstitial fluid (ISF), as well as in the circulating plasma after administration to Sprague Dawley male rats. To do so, rats were implanted with microdialysis probes in the hippocampus and striatum and a jugular vein cannula (JVC). After the collection of one pre-dose ISF sample, animals were dosed with sGC stimulator. Samples were collected for 24 hours through a hippocampal and striatal probe as well as serial plasma sampling collected through the JVC after administration. All collected samples were stored at −80° C. waiting for analysis on compound levels in the dialysate.

### Materials and Methods

### Animals

Five Sprague Dawley rats pre-cannulated with a jugular vein cannula were used for this study. Upon arrival, rats were group-housed in polycarbonate cages (2-3/cage) and acclimated for at least 3 days prior to commencing studies. Animals were housed in a 12 hr light/dark cycle with room temperature maintained at 22+2° C. and approximately 50% humidity and received food and water ad libitum. Rats were tracked via unique identifying numbers. Experiments were conducted in accordance with protocols approved by the Institutional Animal Care and Use Committee of Charles River Laboratories South San Francisco.

### Formulation and Dosing

sGC stimulators were formulated fresh on the day of treatment and administered to animals as follows: Compound I-14 was dosed at a dose of 3 mg/kg, as its sodium salt, and concentration of 3 mg/mL and formulated in MilliQ water.

### In Vitro Experiment

An in vitro Metaquant microdialysis (MQ-MD) experiment was carried out to test the recovery of the compound through the membrane of the probe. To do so, MQ probes (polyacrylonitrile, 3 mm membrane) were connected by the inlet PEEK tubing to a microperfusion pump (Harvard PHD 2000 Syringe pump, Holliston, MA or similar). The probes were individually placed in a bath of artificial CSF (aCSF)+0.2% bovine serum albumin (BSA) containing 50 ng/mL of the sGC stimulator. The bath content was continuously stirred and kept at 37° C. Each probe was perfused with aCSF+0.2% β-cyclodextrin (β-CD, slow flow) and a carrier flow of ultra-pure water+0.2% BSA was used. Flow rates were 0.15 μL/min for the slow flow and 0.8 μL/min for the carrier flow. The outlet of the probe was connected by the outlet PEEK tubing to an automated fraction collector (820 Microsampler, Univentor, Malta or similar). After perfusion stabilization, samples were collected for 20-minute periods into polypropylene vials. In a separate vial, 150 μL bath content samples were collected at the start and at the end of the experimental sample collection period. In vitro dialysates and bath samples were analyzed for compound levels. The probe recovery calculated as the ratio of compound concentration in the dialysate samples over the bath concentration and expressed as a percent recovery.

### Microdialysis Procedure

Rats were anesthetized using isoflurane (2%, 800 mL/min O₂). Bupivacaine was used for local anesthesia and carprofen was used for peri-/post-operative analgesia. Animals were placed in a stereotaxic frame (Kopf instruments, USA). Then, MetaQuant microdialysis probes (polyacrylonitrile; 3 mm exposed membrane) were implanted into the stratium (STR) and hippocampus (HIPP). Coordinates for the tip of the probe for the STR were: antero-posterior (AP)=+0.9 mm from bregma, lateral (L)=+3.0 mm from midline and ventral (V)=−7.0 mm from dura, the toothbar set at −3.3 mm. Then the second probe was implanted into the hippocampus (HIPP). Coordinates for the tip of the probe for the HIPP were: antero-posterior (AP)=−5.3 mm from bregma, lateral (L)=−4.8 mm from midline and ventral (V)=−7.0 mm from dura, the toothbar set at −3.3 mm. After surgery animals were housed individually in cages and provided food and water ad libitum.

Microdialysis experiments were performed one day after surgery. The microdialysis probes were connected with flexible PEEK tubing to a microperfusion pump (Harvard PHD 2000 Syringe pump Holliston, MA). Microdialysis probes were perfused with aCSF containing 147 mM NaCl, 3.0 mM KCl, 1.2 mM CaCl₂ and 1.2 mM MgCl₂ and 0.2% β-Cyclodextrin for the slow flow rate of 0.15 μL/min and Carrier of H₂O+0.2% BSA at a rate of 0.8 μL/min. Microdialysis samples were collected for 30-minute periods by an automated fraction collector (820 Microsampler, Univentor, Malta) into 300 uL polypropylene mini-vials containing 15 μL 0.02M formic acid+0.04% ascorbic acid in ultra-pure water. After stabilization, one baseline sample was collected, and the sGC stimulator was administered orally (PO) at T=0 and samples were continuously collected for 24 hours (collections at 1h, 2h, 3h, 4h, 5h, 6h, 8h, 12h and 24 h). Samples were aliquoted for analysis of sGC stimulator. All ISF samples were stored at −80° C. until analysis was conducted.

In addition to ISF collection, blood samples were collected via the JVC into K₂+EDTA vials at T=−0.5h, 0.5h, 1h, 2h, 3h, 4h, 5h, 6h, 8h, 12h and 24h after treatment. Blood was stored on ice until plasma processing (centrifugation at 4° C., 2,500 g for 10 min). Plasma was aliquoted into 1.5 mL Eppendorf vials and stored at −80° C. awaiting analysis.

### Post-Mortem Tissue Collection

After completion of the microdialysis, animals were euthanized via CO₂ asphyxiation. Brains were collected in 10% neutral buffered formalin for probe placement verification.

### Bioanalytical Methods

### Measuring Compound Levels in Dialysate and Plasma

Concentrations of Compound I-14 in dialysate and plasma samples were quantified by Ultra-high performance liquid chromatography (UPLC) coupled with tandem mass spectrometry (MS/MS) detection in multiple-reaction-monitoring mode (MRM).

Plasma samples were first mixed for protein precipitation with a solution of acetonitrile containing 100 ng/mL of dexamethasone (internal standard). After 5 minutes incubation at room temperature, the samples were centrifuged for 5 minutes (1300 rpm, 4° C.) and the supernatant was diluted 100-fold in ultrapure water with 0.1% formic acid.

Undiluted dialysate samples (10 μL) were mixed with 4 μL of internal standard solution containing 50 ng/mL of dexamethasone (internal standard) dissolved in acetonitrile/ultrapure water (1:1) with 0.1% formic acid prior to analysis.

Undiluted dialysate samples and diluted plasma supernatants were injected into into a Shimadzu system (Shimadzu, USA) by an automated sample injector (Shimadzu Sil-30AC Autosampler, Shimadzu, USA). Analytes were separated by liquid chromatography using a linear gradient of mobile phase B at a flow rate of 0.800 mL/min on a reversed phased XBridge BEH C8 column (2.1*50 mm, 2.5 μm particle size; Waters, USA) held at a temperature of 35° C. Mobile phase A consisted of ultrapure water with 0.1% formic acid. Mobile phase B was acetonitrile with 0.1% formic acid.

Acquisitions were achieved in positive ionization mode using a QTrap® 5500 mass spectrometer (Applied Biosystems, USA) equipped with a Turbo Ion Spray interface. The ion spray voltage was set at 5.5 kV and the probe temperature was 600° C. The collision gas (nitrogen) pressure was kept at medium. The following MRM transitions were used for quantification: m/z 372.0/352.0. Suitable in-run calibration curves were fitted using weighted (1/x) regression, and the sample concentrations were determined using these calibration curves. Accuracy was verified by quality control samples after each sample series. Data were calibrated and quantified using the Analyst™ data system (Applied Biosystems, version 1.5.2).

### Data Evaluation

Data were plotted in Prism 8 for Windows (GraphPad Software, Inc.,). The reported dialysate concentrations of Compound I-14 were corrected based on probe recoveries. Mean Recovery was 14.7% (with a SEM of 0.59)

### Results

Five animals were successfully dosed and complete the experiment. One animal's JVC became blocked at the 4 hour-timepoint and no additional blood was collected. No obvious side effects of the treatment were observed.

### Effect of Administration of Compound I-14 in Striatal and Hippocampal ISF; Ratio of Compound in STI/HIPP Vs Plasma

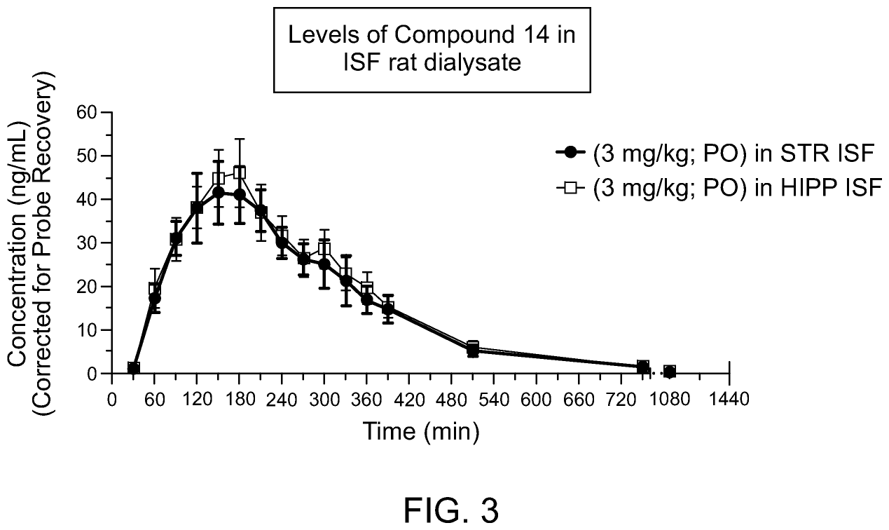

FIG. 3 shows the levels of Compound I-14 in the STR and HIPP portions of ISF dialysate in adult male Sprague-Dawley rats following administration (3 mg/kg; PO) of Compound I-14 at T=0 min.

Microdialysis allows for sampling of compound concentrations in the interstitial fluid of brain tissues. Given that the tissues at the sampling location have a different rate of blood perfusion, physiological makeup, and mechanisms of clearance compared to the cerebrospinal fluid (CSF), we would expect the distribution and measured concentrations to be different between a microdialysis study and CSF-PK study in the same animals. These differences would be observed in the time to maximal concentration, the measured concentrations and the ratios calculated from the measured concentrations (Nagaya Y, Nozaki Y, Takenaka O, et al. Investigation of utility of cerebrospinal fluid drug concentration as a surrogate for interstitial fluid concentration using microdialysis coupled with cisternal cerebrospinal fluid sampling in wild-type and Mdr1a(−/−) rats. Drug Metab Pharmacokinet. 2016; 31(1):57-66).

### Example 9: Concentration of cGMP Changes in Rat Cerebrospinal Fluid (CSF) after a Single Administration of sGC Stimulator (CSF Biomarker Measurement)

This experiment was carried out to determine the effect of different doses of sGC stimulator compounds of the invention on the cGMP levels in rat CSF.

### Protocol

Rats were administered a single dose of either vehicle or sGC stimulator (1, 3, 10 or 30 mg/kg). One, two and six hours after administration, CSF samples were collected and analyzed to determine cGMP and compound concentrations and plasma samples were collected and analyzed to determine compound concentration. Each rat was sampled one time or multiple times with 3 or more days in between each dosing. The day before the experiment rats were fasted overnight with ad libitum access to water.

On the day of the experiment, compound and cyclic guanosine monophosphate (cGMP) concentrations in rat CSF was determined following oral dosing. Male CD rats implanted with an intracisternal cannula (250-275 g) were used for these studies. Rats were single housed under controlled conditions of temperature (21±1° C.), relative humidity (36±1%) and placed in a 12-hr light-dark cycle (lights on at 6:00 AM and off at 6:00 PM) room at SmartLabs vivarium (21 Erie Street, Cambridge, MA) under protocol MIL-110. Animals were allowed ad libitum access to chow (LabDiet Prolab Isopro RMH 3000, St. Louis, MO) and water. The rats were dosed with 0 mg/kg (vehicle), 1 mg/kg, 3 mg/kg, 10 mg/kg, or 30 mg/kg of a compound of the invention formulated as a suspension in 0.5% methylcellulose, 0.5% Tween80 or as a solution in MilliQ water. Formulations were either prepared, stored frozen at −20° C. and thawed at room temperature for 1 hour before dosing or prepared and used within 2 hours or they were prepared the day before dosing and kept stirring overnight at room temperature and until dosing. PO doses were administered by oral gavage and delivered to the stomach using a syringe and gavage tube. Following oral dosage administration, the gavage tube was flushed with approximately 0.5 mL of water to ensure complete delivery of the full dose.

Plasma and CSF samples were collected under isoflurane anesthesia as follows: samples of CSF and blood were collected at 1, 2, and 6 hours post-dosing. CSF samples were collected through the intracisternal catheter. Approximately 20 μL of CSF were collected and discarded (this includes the syringe dead volume is 14-16 μL); then approximately 50 μL of CSF was withdrawn in Eppendorf tubes containing 5 μL of glacial acetic acid. CSF samples were snapped frozen by immersion in liquid nitrogen. Next, animals remained under anesthesia and a blood sample was obtained through a tail nick and stored in a K-EDTA tube. These samples were kept on ice until processed for plasma. Blood samples were centrifuged at 3200 rpm for 10 minutes at approximately 5° C. within 1 hour of collection. Plasma was directly transferred to a 96-well plate tube (0.125 mL). Plug caps were placed on the tubes and the tubes frozen at approximately −70° C. and stored until analysis. Plasma and CSF was collected and analyzed for the presence of a compound.

Quantitation of Compounds and cGMP.

The compound of the invention, cGMP and the internal standard were extracted from plasma and CSF by precipitation. Samples were analyzed using liquid chromatography (LC) with tandem mass spectrometric detection (MS/MS) using electrospray ionization. The standard curve range for compounds ranged from 0.1 to 1000 ng/mL. The standard curve range for cGMP ranged from 0.01 to 40 ng/mL. CSF cGMP data were graphed using Graphpad Prism, version 8.4.3 and are expressed as means±S.E.M. Data were analyzed with a Mixed-effects analysis followed by Dunnett's multiple comparison test comparing to vehicle-treated rats within timepoint. Significance was set at p<0.05.

### Results

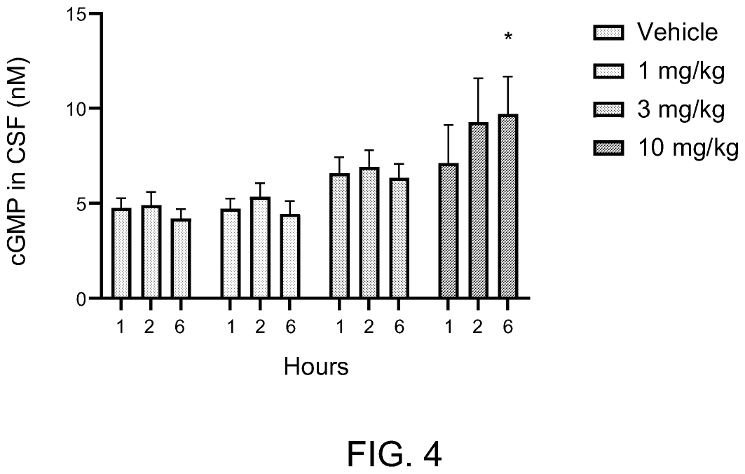

One and two hours after dosing, rats treated with 1, 3 and 10 mg/kg of Compound I-20 (dosed as a suspension in Tween/MC) had no significant change in the concentration of cGMP in rat CSF as compared to vehicle-treated rats. However, at six hours post-dose, rats treated with 10 mg/kg of Compound I-20 had significantly higher cGMP in the CSF than vehicle-treated rats. (see FIG. 4)

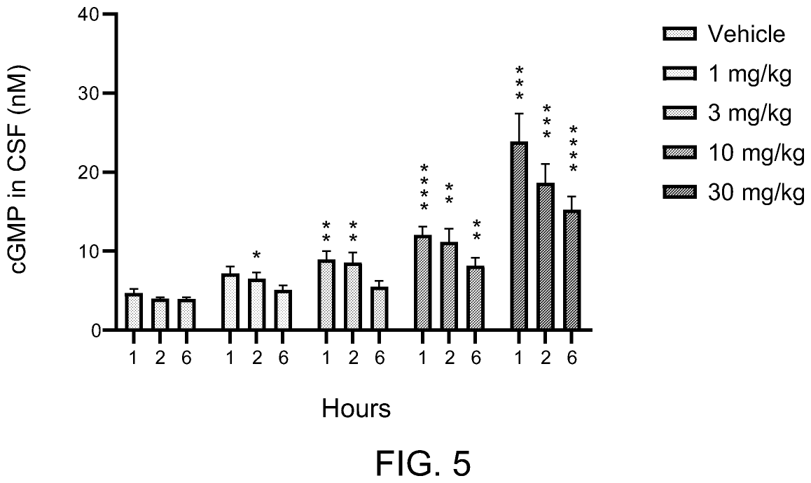

Rats treated with Compound I-14 (administered as the sodium salt) had higher levels of cGMP in the CSF at all tested doses, but not at all timepoints tested, as compared to vehicle-treated rats. One and two hours after dosing, rats administered 3 mg/kg Compound I-14 had significantly higher concentrations of cGMP in the CSF. Two hours after dosing, rats treated with 1 mg/kg Compound I-14 had significantly higher cGMP in CSF as compared to vehicle-treated rats. Rats administered either 10 or 30 mg/kg Compound I-14 had significantly higher cGMP in the CSF at 1, 2 and 6 hours postdose (see FIG. 5)

### Example 10A: Non-Human Primate Cerebrospinal Fluid (CSF) Pharmacokinetic Properties (Study A)

### Protocol.

PK in NHP was determined following oral dosing (PO). A group of 4 female Cynomolgus monkeys were used for studying each compound. Compound I-20 sodium salt was formulated as a 0.06 mg/mL solution in MilliQ water. Compound I-14 sodium salt was formulated as 0.2 mg/mL solution in MilliQ water. Formulations were shipped frozen on dry ice and then thawed and mixed thoroughly prior to dosing. PO doses of 1 mg/kg for Compound I-14 and 0.3 mg/kg for Compound I-20 were administered by oral gavage.

Plasma and CSF samples were collected as follows: samples of CSF were collected at 3 and 24 hours post PO dosing. CSF samples (0.125 mL) were collected at the cisterna magna via direct needle puncture by direct dilution. Blood samples (0.8 mL) were collected from a peripheral vein at 0, 0.25, 0.5, 1, 2, 3, 6, 8, 2, 24, 32 and 48 or 72 hours. These samples were kept on ice until processed for plasma. Blood samples were protein precipitated using acetonitrile. Plasma was directly transferred to individual tubes (0.125 mL) and K₂EDTA was used as the anticoagulant. Plug caps were placed on the tubes and the tubes frozen at approximately −70° C. and stored until analysis. Plasma and CSF was collected and analyzed for the presence of compound.

### Quantitation of Compounds.

Plasma and CSF samples were analyzed using liquid chromatography (LC) with tandem mass spectrometric detection (MS/MS) using positive electrospray ionization. The standard curve range was from 0.1 to 1000 ng/mL.

The geometrical means for the values obtained for the 4 animals in each compound's study was obtained for concentrations in CSF and in Plasma, respectively.

Kp,uu is defined as the concentration ratio of unbound drug in CSF to unbound drug in plasma. Unbound drug in plasma (or free plasma concentration) is calculated by multiplying the total plasma concentration by the unbound fraction as determined by plasma protein binding. The CSF concentration is then divided by the free plasma concentration to determine the Kp,uu. (See e.g., Di et al., J. Med. Chem., 56, 2-12 (2013))

Results for compounds of the invention are summarized Table H below.

TABLE H

| Compound | Dose | Kp, uu | Kp, uu |
|---|---|---|---|
| num | (mg/kg) | at 3 h | at 24 h |
| I-20 | 0.3 | A | B |
| I-14 | 1 | C | D |

NHP CSF PK. Kp, uu ≤ 1 = A; 1 < Kp, uu ≤ 2 = B; 2 < Kp, uu ≤ 3 = C, 3 < Kp, uu = D.

### Example 10B: Non-Human Primate Cerebrospinal Fluid (CSF) Pharmacokinetic Properties (Study B)

The objective of this study was to investigate the pharmacokinetics of Compound I-14 after administration of a single intrathecal bolus or oral gavage dose to cynomolgus monkeys on Day 1 and to evaluate differences in pharmacokinetic values when comparing CSF collected at the cisterna magna (as in Example 10A) versus lumbar locations.

### Study Design

| | | | Dose | Dose | Dose | Dose | |
|---|---|---|---|---|---|---|---|
| Group | No. of | | Level | Concentration | Volume | Route/ | |
| No. | Animals^{a} | Test Material | (mg/kg) | (mg/mL) | (mL) | Regimen | Collections |
| 1 | 4 F | Compoound I-14 | 1 total | 0.1/0.1 | 5 | Single Oral | Blood at 15 and |
| | | | | | | Gavage dose | 30 min.; 1, 2, 3, |
| | | | | | | of each test | 6, 8, 12, 24, 32, |
| | | | | | | article on | & 48 hours post |
| | | | | | | Day 1 | dose |
| 2 | 4 F | | 0.3 total | 0.03/0.03 | 5 | Single IV | CSF at 3 and 24- |
| | | | | | | bolus of | hours post dose |
| | | | | | | each test | (CSF collected |
| | | | | | | article on | via Cisterna |
| | | | | | | Day 1 | Magna direct |
| | | | | | | | stick) |
| 3 | 4F | | 1 total | 0.1/0.1 | 5 | Single Oral | Blood at 15 and |
| | | | | | | Gavage dose | 30 min.; 1, 2, 3, |
| | | | | | | of each test | 6, 8, 12, 24, 32, |
| | | | | | | article on | & 48 hours post |
| | | | | | | Day 1 | dose |
| 4 | 4 F | | 0.3 total | 0.03/0.03 | 5 | Single IV | CSF at 3 and 24- |
| | | | | | | bolus of | hours post dose |
| | | | | | | each test | (CSF collected |
| | | | | | | article on | via Lumbar Port) |
| | | | | | | Day 1 | |

No. = Number; F = Female; CSF = cerebrospinal fluid; No = Number^{a}
The same animals will be used for each group, there will be a minimum of 7 days washout between dosing days for each group

### Test System/Methods

| | |
|---|---|
| Species: | Macaca fascicularis |
| Strain: | Cynomolgus macaque |
| Number of Females: | 4 |
| Age: | Adult |
| Weight: | 2.5-4 kg |

Animals were housed in stainless steel cages equipped with a stainless steel mesh floor and an automatic watering valve. Primary enclosures were as described in the Guide for the Care and Use of Laboratory Animals (National Research Council (NRC). Guide for the Care and Use of Laboratory Animals. Washington, D.C: National Academy Press, 8th Ed. 2011; Office of Laboratory Animal Welfare. Public Health Services Policy on Humane Care and Use of Laboratory Animals. Bethesda, Maryland: National Institutes of Health, Revised 2015). These housing conditions were maintained unless deemed inappropriate by the Study Director and/or Clinical Veterinarian. Animals were socially housed, when possible, with the exception of times when they were separated for designated study procedures/activities.

Single doses of Compound I-14 were administered intravenously as a bolus injection of a solution at 0.15 mg/kg and orally by gavage as a suspension at 0.5 mg/kg to a group of four female cynomolgus monkeys in a crossover design. Plasma samples were collected at 0.25 (15 min), 0.5 (30 min), 1, 2, 3, 6, 8, 12, 24, 32, and 48 h following the IV and PO doses. CSF samples were collected at 3 and 24 h for all animals from the cisterna magna in groups 1 and 2 and the lumbar from groups 3 and 4. Sample extracts were prepared by protein precipitation and Compound I-14 concentrations were measured using LC-MS/MS. Pharmacokinetic parameters were calculated.

### Summary/Conclusions

Overall, the mean pharmacokinetic parameters between IV (Groups 2 and 4) and PO (Groups 1 and 3) were within the standard deviation of each other.

Compound I-14 was observed in the CSF at 3 and 24 hours following both IV and PO administration using both cisterna magna and lumbar sampling. At 3 hours following IV and oral dosing, geomean ratio of CSF concentration to unbound concentration in plasma fell in both cases within the range C as described above, i.e. between 2 and 3 (NHP CSF PK. Kp,uu≤1=A; 1<Kp,uu≤2=B; 2<Kp,uu≤3=C, 3<Kp,uu=D), following sampling from the cisterna magna and also following lumbar sampling. At 24 hours following IV and oral dosing, geomean ratio of CSF concentration to unbound concentration in plasma fell in range B and C, respectively, following sampling from the cisterna magna and B and C, respectively, following lumbar sampling.

### Example 11: Evaluation of Compounds of the Invention in the Novel Object Recognition (NOR) Model of Memory Enhancement

### Objective

To assess the efficacy of CNS penetrant sGC stimulators of the invention in reversing memory disruption induced by MK-801 using the Novel Object Recognition (NOR) test in male Long Evans rats.

### Introduction

The NOR is a test of recognition learning and memory retrieval, which takes advantage of the spontaneous preference of rodents to investigate a novel object compared with a familiar one. The NOR test has been employed extensively to assess the potential cognitive-enhancing properties of novel test compounds. Because the NOR paradigm does not involve reward or noxious stimuli, it provides fewer confounding variables when being translated into analogous tests conducted in human clinical trials.

In the present study, a memory saving model was used. MK-801 (Dizocilpine), an uncompetitive antagonist of the NMDA receptor was used to cause deficit of recognition memory. sGC stimulators were evaluated for their efficacy to prevent memory impairment induced by MK-801. Reference compound galantamine 1 mg/kg (i.p.) significantly reversed the cognitive deficit induced by MK-801 0.1 mg/kg (i.p.), suggesting the validity of the test.

### Materials and Methods

### Animals

Adult male Long-Evans rats (275-299 gram at arrival from Envigo, Indianapolis, IN) were used in this study. Rats were placed in the experimental rooms and assigned unique identification numbers (tail marks). Rats were housed 2 per cage in polycarbonate cages with filter tops and acclimated for at least 7 days prior to testing. Animal room was maintained in a 12/12 h light/dark cycle (lights on at 07.00 EST), 22±1° C. and relative humidity at approximately 50%. Food and water were provided ad libitum. All animals were examined, handled and weighed prior to the study to assure adequate health and to minimize the non-specific stress associated with testing. Each animal was randomly assigned across the treatment groups. The experiments were conducted during the animal's light cycle phase.

### Test Compounds and Agents

The following compounds and agents were used in these studies:

MK-801 (0.1 mg/kg; Sigma-Aldrich) was dissolved in saline and injected IP, 15 min prior to NOR training. The dose volume was 1 ml/kg.

Galantamine (1 mg/kg; Tocris) was dissolved in saline and injected IP, 15 minutes prior to training. The dose volume was 1 ml/kg.

Compound I-20 (0.03, 0.3 and 1 mg/kg) was formulated in vehicle (0.5% (w/w) methyl cellulose and 0.5% (w/w) Tween 80 in ultrapure water) and orally dosed 60 min prior to NOR training at 2 ml/kg dose volume.

The following groups were tested, with N=16 in each (One rat was removed from the Compound I-20-1 mg/kg-MK-801 group before test started due to a health issue): 1) Vehicle—Saline; 2) Vehicle—MK-801 0.1 mg/kg; 3) Galantamine 1 mg/kg—MK-801; 4) Compound I-20, 0.03 mg/kg—MK-801; 5) Compound I-20, 0.3 mg/kg—MK-801; and 6) Compound I-20, 1 mg/kg—MK-801

Compound I-14 (0.01, 0.1 and 1 mg/kg) was formulated as its Na+ salt as a solution in MilliQ water and stored as frozen aliquots. The aliquots of compound solutions and compound vehicle were stored in −80° C. and freshly thawed on each testing day. The compound and vehicle were orally dosed 60 minutes prior to NOR training. The dose volume was 10 ml/kg.

The following groups were tested for Compound I-14, with N=16 in each: 1) Saline—MK-801 0.1 mg/kg; 2) Galantamine 1 mg/kg—MK-801; 3) Vehicle—Saline; 4) Vehicle—MK-801; 5) Compound I-14, 0.01 mg/kg—MK-801; 6) Compound I-14, 0.1 mg/kg—MK-801; and 7) Compound I-14, 1 mg/kg—MK-801

### Experimental Procedures

NOR test was conducted in an open-field arena (40×40 cm) placed in a sound-attenuated room under dimmed lighting. Each rat was tested separately, and care was taken to remove olfactory/taste cues by cleaning the arena and test objects with 70% alcohol between trials and rats. All training and testing trials were video-taped and scored by an observer blind to treatments.

On Days 1 and 2, rats were allowed to freely explore the arena (no objects inside) for a 5-minute habituation period. On Day 3 (training and testing day), rats were administered vehicle, saline and/or compound solution according to corresponding pretreatment, which is defined as time between injection and start of NOR training. Each animal was placed into the test arena in the presence of two identical objects. Each rat was placed in the arena facing the same direction at the same position, and the time spent actively exploring the objects during a 3-minute training period (T1) was recorded. The rat was returned to its home cage following training. NOR test (T2) was conducted 1 hour after T1. Each rat was placed back into the test arena in the presence of one familiar object and one novel object for 5 minutes, and the time spent exploring both objects was recorded. The presentation order and position of the objects (left/right) in T2 was randomized between rats to prevent bias from order or place preference.

### Tissue Collections

About 10 minutes after T2 (135 minutes after drug administration), trunk blood was collected in microcentrifuge tubes containing K2EDTA. The blood tubes were kept on ice for short term storage. Within 15 minutes the tubes were centrifuged for 10 minutes at 10,000 RPM in a refrigerated centrifuge. Plasma was extracted and samples are stored in the −80° C. freezer until shipment to Sponsor.

### Statistical Analysis

Data of NOR test (T2) were expressed as Recognition Index, which is defined as the ratio of the time spent exploring the novel object over the total time spent exploring both objects (Novel/(Familiar+Novel)×100%) during the test session.

For Compound I-14, data were analyzed in two batches separately. Batch 1 includes Saline-MK-801 group and Galantamine-MK-801 group. Data were analyzed with t-test to evaluate the validity of the test. Batch 2 includes the “sGC stimulator Groups” including 5 treatment groups which contain compound vehicle (MilliQ water): Vehicle-Saline, Vehicle-MK-801, Compound I-14, 0.01 mg/kg—MK-801, Compound I-14, 0.1 mg/kg—MK-801 and Compound I-14, 1 mg/kg—MK-801 or. Batch 2 data were analyzed by one-way ANOVA followed by Fisher LSD post hoc comparisons in 0-1, 0-3 and 0-5 minute time range separately. The significance was set at P<0.05. Nineteen animals with Recognition Index above 90% or below 30% were eliminated because they suggest strong (non-memory) bias between two objects. Two rats whose overall exploration time to both objects in five minutes less than 10 seconds were also eliminated due to unreliable results (This is a standard criterion of our NOR test.) One rat was removed due to questionable drug exposure based on the feedback of plasma analysis. And then statistical outliers that fell above or below two standard deviations from the mean were removed from the further analysis. With these criteria, 1-6 rats were eliminated from each experimental group (N=16 originally) and were excluded from statistical analyses for all time range (0-1, 0-3, and 0-5 minute).

For Compound I-20, data were analyzed by using one-way ANOVA followed by Fisher's LSD post hoc test on 0-1, 0-3 and 0-5 minute time range separately, with significance set at P<0.05. Animals with recognition index above 90% or below 30% were eliminated because they suggest strong (non-memory) bias between two objects. And then statistical outliers that fell above or below two standard deviations from the mean were removed from the further analysis. With these criteria, 2-4 rats were eliminated from each experimental group (N=16 originally) and were excluded from statistical analyses for all time ranges (0-1, 0-3, and 0-5 minute).

### Results

### a) Compound I-14

None of the rats in this study showed obvious side effects at any dose. Rats maintained normal vigilance, activity and exploration level to objects.

In the 0-1 minute time range, t-test showed significant different between Saline-MK-801 group and Galantamine-MK-801 group (P<0.01), indicating the validity of this assay. ANOVA to the Compound Group showed a significant main treatment effect on Recognition Index [F(4,56)=4.698, P<0.01]. Fisher LSD post hoc comparison indicated that at this time range Vehicle-Saline group and the 1 mg/kg Compound I-14—MK-801 group both showed significant difference from Vehicle-MK-801 group (P<0.05 and P<0.01, respectively), suggesting MK-801 0.1 mg/kg induced significant memory deficit and Compound I-14 at 1 mg/kg reversed the deficit.

In the 0-3 minute time range, t-test showed significant different between Saline-MK-801 group and Galantamine-MK-801 group (P<0.001), indicating the validity of this assay. ANOVA to the Compound Group showed a significant main treatment effect on Recognition Index [F(4,56)=5.113, P<0.01]. Post hoc comparison indicated that in this time range Vehicle-Saline group showed significant difference from Vehicle-MK-801 group (P<0.01), suggesting MK-801 0.1 mg/kg induced significant memory deficit. In this time range, Compound I-14 at 1 mg/kg showed a trend (P<0.10) in reversing MK-801-induced memory deficit.

In the 0-5-minute time range, t-test showed significant different between Saline-MK-801 group and Galantamine-MK-801 group (P<0.001), indicating the validity of this assay. ANOVA to the

Compound Group showed a significant main treatment effect on Recognition Index [F(4,56)=2.847, P<0.05]. Post hoc comparison showed that at this time range Compound I-14 at 1 mg/kg had significantly higher Recognition Index relative to Vehicle-MK-801 group (P<0.05).

### b) Compound I-20

None of the rats in this study showed obvious side effects at any dose. Rats maintained normal vigilance, activity and exploration level to objects.

ANOVA showed significant main treatment effects on recognition index in 0-1 min time range [F(5,77)=3.379, P<0.01]. Post hoc test showed that in this time range Vehicle-MK-801 0.1 mg/kg group and Vehicle-Saline group did not show significant difference (P>0.05). Compound I-20 1 mg/kg-MK-801 group and Galantamine-MK-801 group showed significantly higher recognition index than Vehicle-MK-801 group (Ps<0.01). Typically, the data from 0-1 min time range are relatively unstable; the data from 0-3 min and 0-5 min time ranges provide more reliable results.

In 0-3 min time range, ANOVA found significant main treatment effects on Recognition Index [F(5,77)=3.922, P<0.01]. Post hoc test showed that in this time range MK-801 0.1 mg/kg caused significant memory deficit (Vehicle-Saline group vs. Vehicle-MK-801 group, P<0.05). Compound I-20 1 mg/kg-MK-801 group and Galantamine-MK-801 group significantly reversed MK-801-induced memory deficits (Ps<0.001). Compound I-20 0.3 mg/kg-MK-801 group also showed a trend (P<0.10) of reversing MK-801-induced memory deficits in this time range.

In 0-5 min time range, ANOVA showed a significant main treatment effect [F(5,77)=5.219, P<0.001]. Post hoc test showed that MK-801 0.1 mg/kg caused a significant memory deficit, with the recognition index approaching chance level (50%). Galantamine (1 mg/kg), Compound I-20 0.3 mg/kg and 1 g/kg significantly reversed MK-801-induced memory deficits (Ps<0.05, P<0.01 and Ps<0.001, respectively, compared to Vehicle-MK-801 group). Compound I-20 0.03 mg/kg-MK-801 group also showed a trend (P<0.10) of reversing MK-801-induced memory deficits in this time range.

### Example 12: Evaluation on Sleep-Wake Pharmaco-EEG in Telemeter-Implanted Rats

This study was conducted at PsychoGenics, Inc. Procedures were approved by the Institutional Animal Care and Use Committee in accordance with the National Institute of Health Guide for the Care and Use of Laboratory Animals.

### Objective

This study was designed to assess the sleep-stage specific pharmaco-EEG (electroencephalography) signatures of Compound I-14 in wirelessly implanted young adult male SD rats.

### Materials and Methods

### Animals

Young adult male Sprague-Dawley (SD) rats (˜275-325 grams on arrival) from Envigo (Indianapolis, IN) were used in the study. Upon receipt, rats were assigned unique identification numbers and housed with 3 rats per cage in polycarbonate cages with micro-isolator filter tops. All rats were examined and weighed prior to initiation of the study to assure adequate health and suitability. During the course of the study, 12/12 light/dark cycles were maintained. The room temperature was maintained between 200 and 23° C. with a relative humidity maintained around 50%. Chow and water were provided ad libitum for the duration of the study. Following surgery, rats were single housed. After a period of recovery (7-10 days), animals were transferred to an EEG recording room and placed over a DSI receiver for recordings.

### Study Arms

a. Vehicle A
   i. Route: PO
   ii. Volume: 10 ml/kg
   iii. Formulation: Water
b. Compound I-14 at 3 mg/kg
   i. Route: PO
   ii. Volume: 10 ml/kg
   iii. Formulation: 3 mg/kg in water
c. Compound I-14 at 10 mg/kg
   i. Route: PO
   ii. Volume: 10 ml/kg
   iii. Formulation: 10 mg/kg in water
d. Compound I-14 at 30 mg/kg
   i. Route: PO
   ii. Volume: 10 ml/kg
   iii. Formulation: 30 mg/kg in water

### Surgical Procedures

Briefly, animals were implanted with DSI Telemetry devices (F50-EET) in a 3 channel (lead pairs) configuration. Note the positive and negative lead placements for each location. Each of the EEG channels on the DSI transmitter functions as a differential input whereby the differential input measures the difference between the positive and negative leads. Frontal/Parietal (right): (+) 2 mm Anterior, 2 mm Lateral; (−) 4 mm Posterior, 2 mm Lateral; Frontal/Frontal (bihemispheric): (+) 2 mm Anterior, 2 mm Lateral (right); (−) 2 mm Anterior, 2 mm Lateral (left); and neck EMG.

In summary, animals were anesthetized with 4 to 5% isoflurane and 1 L/min oxygen for induction prior to surgery and were maintained during surgery with 1-2.5% isoflurane/1 L/min oxygen mix. Depth of anesthesia was determined and monitored using the hindlimb withdrawal reflex and respiratory rate. Isoflurane levels were continually adjusted to maintain a surgical plane of anesthesia throughout the surgical procedure. Ophthalmic lubricant was applied on the eyes with a sterile cotton-tipped swab. The surgical site was prepared for aseptic surgery by shaving the fur and cleaning the skin with Chlorhexidine scrub and alcohol swabs three times. The abdomen, neck, and head were shaved and disinfected with 3 alternating preps of Chlorhexiderm Scrub and alcohol (2% chlorhexidine gluconate and 4% isopropyl alcohol). Anesthetized animals were then placed on a warm water circulating heating pad for surgical implantation of the telemetry devices.

### Telemetry Implant

A 3-4 cm incision was made at the midline on the top of the skull extending from about 1 cm posterior to the midpoint of the eyes to the base of the skull and extending to the mid dorsal neck region. Using blunt dissection, a subcutaneous pocket was formed from the posterior end of the incision by pushing aside connective tissue to the right lateral flank region. The pocket was irrigated with sterile saline. The transmitter was then placed in the pocket with the lead wires extending out of the neck incision. One lead wire, attached to the transmitter, was measured, cut to size and inserted in one of the dorsal neck muscles and secured in place with 5-0 silk suture. This procedure was repeated for a second lead wire so that two leads were placed in-line along the same muscle bundle and spaced 2-4 mm apart and provides electromyography (EMG) signals. The periosteum was then removed to reveal skull landmarks, bregma and lambda. As needed, sterile saline was used to wipe the skull area which was subsequently dried using gauze or sterile Q-tips. Stereotaxic coordinates for areas of interest (posterior and parietal cortex) were used to mark electroencephalographic (EEG) leads. For each area of interest, 1-2 holes were drilled through the skull ensuring exposure of dura. Dural contact screws were inserted into the holes and the EEG leads wrapped around them (maximum of 4 holes). These screws must make contact with the dura to detect EEG brain signals. Penetration through the dura will cause no adverse event and may improve the EEG signal (more brain in contact with metal). The screws and surrounding skull surface area were then permanently sealed using FLOW-It ALC composite. After the cement thoroughly dried, the skin overlying the skull was closed using suture staples.

### Testing Schedule

This study followed a Monday and Thursday dosing/recording schedule, where Baseline EEG was recorded 2 hours and then rats received first dose of Compound I-14 at 0.3, 3 or 10 mg/kg or Vehicle A (all orally) between 7:50-8:00 AM and a second dose of the same after about 12 hours, and EEG continued 12 hours afterwards.
EEG Recordings began at 6 AM (at time lights turn on)
Dosing occurred at 7:50 am AM (1 hour 50 minutes after lights ON)
2^{nd} dosing (same compound) occurred at 7:50 PM (1 hour and 50 minutes after lights OFF)
EEG Recordings continued until 7:50 AM the following day (24 hours after 1^{st} dose and 12 hours after 2^{nd} dose).

Data were recorded using a Data Sciences International (DSI) data acquisition platform from freely moving rats in their home cages. Recordings were conducted during the light and dark cycles. Lights were maintained at 6 AM lights on and 6 PM lights off for the duration of the study. Animals were habituated to dosing (vehicle dosing) before data collection took place.

Animals were tested in a cross-over design with at least 72-hour washout period between doses. On each testing day, animals received compound or vehicle orally just under 2 hours (7:50 AM) after lights on (6:00 AM). Data were recorded from 2 hours prior to dosing and recorded continuously for 24 hours post dose.

### EEG Signal Assessment

EEG was ensured to not contain line noise (50 or 60 Hz) or any continuous, non-physiological frequency pattern that would be considered noise from external powered sources. All EEG recordings were within normal range of operation (i.e. EEG signals fell within normal amplitude of operation, typically greater than 100 microvolts and less than 500 microvolts with no loss of signal fidelity, typically observed by a reduction in EEG, <100 μV).

### Sleep Scoring

Raw EEG recordings were manually scored using Neuroscore software (Data Sciences International) to identify sleep stages: Active Wake, Quiet Wake, NREM and REM. Using NeuroScore (DSI), artifacts were removed offline from the data and sleep stages assigned manually for every 10 s epoch using EEG, EMG and Locomotor activity (LMA) by conventional methods as previously described (Ivarsson et al., 2005; Parmentier-Batteur et al., 2012; Leiser et al., 2014, 2015) using the fronto-parietal EEG, LMA and EMG: active wake (less regular, low-amplitude EEG with high EMG and LMA activity); quiet wake (less regular, low-amplitude EEG, with low EMG and no LMA activity); NREM (consisting of high-amplitude irregular waves with predominant delta (1-4 Hz), low EMG and no LMA); REM sleep (stable, low-amplitude waves dominated by theta (4-8 Hz) with near absent EMG and no LMA).

The sleep stage data were exported from a Neuroscore report template of sleep state time per each 15 min (2 hours pre-dose to 4 hours post-dose). The onset of the first sleep and first REM were also reported directly from a Neuroscore report template. Latency was calculated as the time period from the onset of the first REM bout following the onset of NREM (i.e. T_{REM}−T_{NREM}=REM Latency). Hypnograms were prepared using the percent time spent in each sleep stage per hour time bins. Time in each sleep state was calculated as a percent of total time in each sleep state (mean±standard error of the mean, SEM). The data for individual animals were arranged by treatment group, sleep state, bin and exported to GraphPad PRISM for statistics and graph illustration.

### Spectral Analysis

Spectral analysis was performed using Matlab. The time domain signal collected for multiple channels was collected into DSI/Neuroscore and then the EDF files were transferred to Matlab. Excel files, marking specific timestamps of baseline and post dose, were also transferred to Matlab for time-locking. In Matlab, the power spectral density (PSD) was computed using the Welch method. Next, both raw and relative spectral power was computed for each of the six frequency bands (Delta, Theta, Alpha, Beta, Low-Gamma and High Gamma) and for each 1 Hz sub band. 2 hours of data recorded prior to compound dosing were pooled and defined as “baseline.” Percent change from baseline was calculated based on each channel, subject, dose level, and spectral band and time segment. The average raw, relative and percent change for each frequency band were calculated for each group. Spectral analysis included quantifying the raw, relative, and percent change spectral power for the traditionally defined EEG bands (Delta-0.5-3.9 Hz, Theta-4-7.9 Hz, Alpha-8-11.9 Hz, Beta 12-29.9 Hz, Low Gamma 30-49.9 Hz, and High Gamma 50-100 Hz) per recording per rat. Additionally, 1-100 Hz EEG spectra were represented in line plots. These spectral plot data were provided to the client separately and not all are included herein as the number of graphs are extensive. For clarity, the percent change in each band is presented over the time of the post dose period.

### Results

### Sleep

The hypnograms for Compound I-14 showed a notable decrease in REM and NREM in the high dose (30 mg/kg) treatment group compared to the vehicle (A) group. An increase in Quiet Wake was observed in the compound group at the highest dose. The onset of NREM, REM and REM latency was delayed in this treatment group. The most profound effects of Compound I-14 occurred in the high dose with effects in Quiet wake (increase), REM (decrease), and NREM (decrease) lasting several hours after dosing.

### Spectral Analysis

Compound I-14 (3 mg/kg) increased low gamma 0-180 minutes post dose, while at 30 mg/kg dose, it increased low gamma 0-240 and high gamma 0-180 minutes post dose in QW. At 30 mg/kg, the compound decreased delta, theta, alpha and increased low and high gamma ˜0-300 minutes post dose in NREM.

### Example 13: Evaluation of the Cognitive Effects of Compound I-14 in the Chronic Low Dose MPTP-Lesioned Macaque Model of Cognitive Deficits in Parkinson's Disease

This study was a non-GLP study in the chronic low dose MPTP-lesioned macaque model of Parkinson's disease. This model has been described in the literature (https://www.ncbi.nlm.nih.gov/pmc/articles/PMC3282499/). The study was designed to assess effects of Compound I-14, administered as monotherapy, on performance of the following cognitive tasks: variable delayed response (VDR), continuous performance (CPT), visual discrimination reversal learning (SDR) and object retrieval (OR). The study was conducted in animals that had received prior treatment with low doses of MPTP and were known to have difficulties in the performance of the above-mentioned tasks. These studies utilised four male cynomolgus macaques with MPTP-induced cognitive deficits.

The study had 4 parts. The first part of the study was to collect baseline data from the four animals on the four tasks to be studied. The second part of the study was to administer vehicle (sterile, filtered distilled water) over a 16 day period and collect vehicle data on the four tasks once per week for two weeks. In the third part of the study, Compound I-14 was administered orally daily for five days and then data were collected on the four tasks once per week for two weeks, with continued daily administration of Compound I-14 (3.0 mg/kg) 2 hrs. prior to each days testing. In the final part of the study, there was a 9 day washout period and then data were collected on the four tasks once per week for two weeks. One dose of Compound I-14 was assessed as monotherapy (3.0 mg/kg) following five days of pre-dosing and daily dosing for two weeks. The effects of the treatment on cognitive performance was assessed 2 hrs. after daily drug administration and during drug washout.

MPTP-HCl was administered by i.v. injection at doses ranging from 0.05 mg/kg to 0.30 mg/kg, 2 to 3 times per week for several months. MPTP administration continued until cognitive deficits appeared with minimal/mild parkinsonian motor impairment. Animals were considered “cognitively impaired” if they show at least a 15% decrease in cognitive task performance from pre-MPTP baseline levels. Because response to MPTP is somewhat idiosyncratic, MPTP was administered to effect (i.e., development of cognitive deficits and then development of motor deficits), rather than given for a particular duration of exposure or a particular cumulative dose of MPTP.

SDR represents a test of cognitive flexibility. In this task, three stimuli are presented at the same time on the screen and one of these stimuli is arbitrarily designated as the positive (rewarded) stimulus and touching it results in a positive tone and reward while touch of a negative (non-rewarded) stimulus results in a different tone and the screen blanks. The positions of the stimuli on the screen vary pseudo-randomly from trial to trial. A maximum of 300 trials are presented in each session. The measure recorded for each session is: 1) total number of trials needed to learn the initial discrimination (i.e. reach the specified criterion of 14/16 correct, and; 2) total number of trials needed to learn (i.e., reach the same criterion as mentioned above) the reversal in which the previously negative (unrewarded) stimulus is now the positive (rewarded) stimulus (i.e., reach criterion). The number of trials needed to reach criterion on learning the discrimination reversal is taken as a measure of cognitive flexibility.

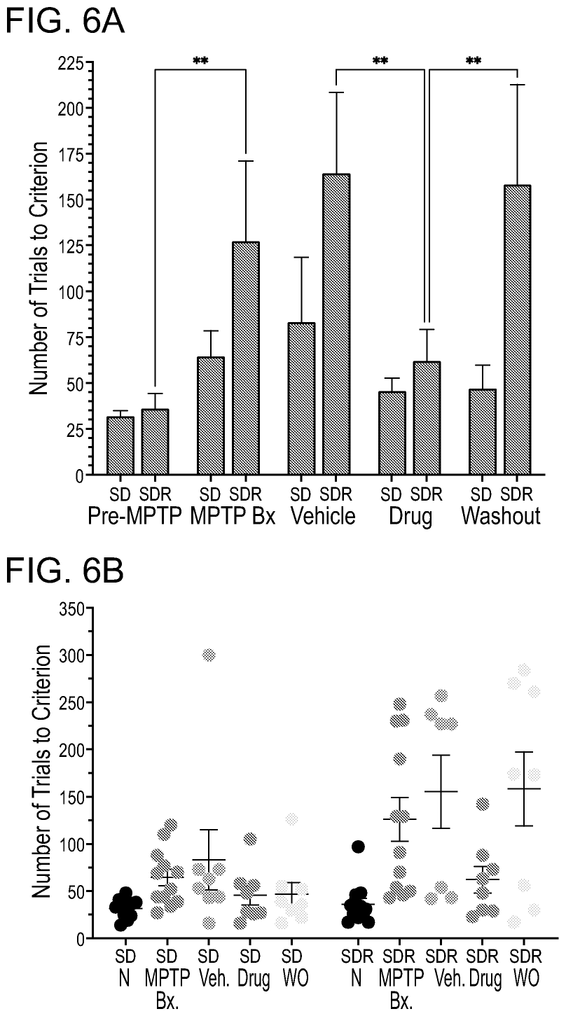

Compound I-14 monotherapy significantly improved performance in the SDR paradigm. CY3018 significantly reduced the number of trials needed to reach criterion to learn the discrimination reversal, which points to improvement in at least one aspect of cognition in these animals. Simple discrimination performance was not significantly changed by Vehicle or Compound I-14 administration. However, Compound I-14 administration resulted in a significant improvement in discrimination reversal learning performance (FIG. 6).

In addition, during Baseline, Vehicle and Washout testing, some animals failed to successfully learn the discrimination reversal, however, when tested with Compound I-14, none of the animals failed to learn the reversal, showing that the compound had a positive effect on all animals tested.

1. A method of treating a CNS disease, health condition or disorder in a subject in need thereof, comprising administering to the subject a therapeutically effective amount of a compound of Formula I: 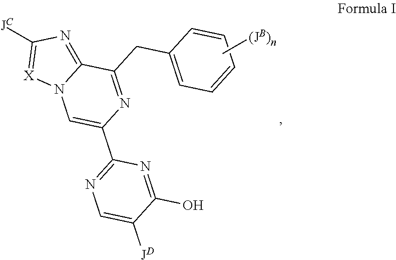
or a pharmaceutically acceptable salt thereof, wherein:
J^{C} is selected from the group consisting of hydrogen, halogen, C₁₋₆ alkyl and C₁₋₆ fluoroalkyl substituted with 1 to 3 fluoro atoms;
X is N or C(J^{C1});
J^{C1} is selected from the group consisting of hydrogen, halogen, C₁₋₆ alkyl and C₁₋₆ fluoroalkyl substituted with 1 to 3 fluoro atoms;
each J^{B} is independently selected from the group consisting of hydrogen, halogen, C₁₋₆ alkyl and C₁₋₆ fluoroalkyl substituted with 1 to 3 fluoro atoms;
J^{D} is selected from the group consisting of hydrogen, halogen, C₁₋₆ alkyl and C₁₋₆ fluoroalkyl substituted with 1 to 3 fluoro atoms; and
n is an integer selected from 0, 1, 2, 3 or 4.

2. The method of claim 1, wherein:
J^{C} is selected from the group consisting of hydrogen, halogen and C₁₋₆ alkyl;
X is N or C(J^{C1});
J^{C1} is selected from the group consisting of hydrogen, halogen and C₁₋₆ alkyl;
each J^{B} is independently selected from the group consisting of hydrogen, halogen and C₁₋₆ alkyl;
J^{D} is selected from the group consisting of hydrogen, halogen and C₁₋₆ alkyl; and
n is an integer selected from 0, 1, 2, 3 or 4.

3. The method of claim 1 or 2, wherein the compound is represented by Formula IA: 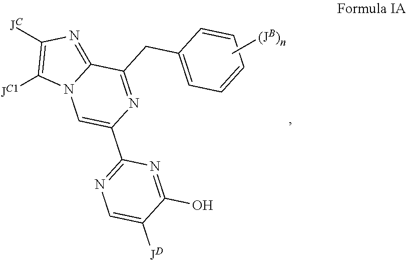
or a pharmaceutically acceptable salt thereof.

4. The method of any one of claims 1-3, wherein J^{C1} is H, F or Cl.

5. The method of any one of claims 1-3, wherein J^{C1} is H.

6. The method of any one of claims 1-3, wherein J^{C1} is F.

7. The method of claim 1 or 2, wherein the compound is represented by Formula IB: 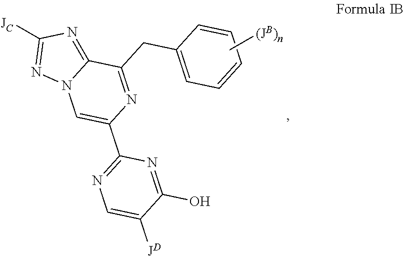
or a pharmaceutically acceptable salt thereof.

8. The method of any one of claims 1-7, wherein n is 2 or 3.

9. The method of any one of claims 1-7, wherein n is 0 or 1.

10. The method of any one of claims 1-9, wherein each J^{B} is independently H, F or C₁₋₄alkyl.

11. The method of claim 10, wherein each J^{B} is independently F or methyl.

12. The method of claim 8, wherein n is 2 and J^{B} are both F or one of J^{B} is F and the other is methyl.

13. The method of claim 8, wherein n is 3 and two of J^{B} are F and the other is methyl.

14. The method of claim 9, wherein n is 1 and J^{B} is F.

15. The method of claim 9, wherein n is 0.

16. The method of any one of claims 1-15, wherein J^{D} is hydrogen.

17. The method of any one of claims 1-15, wherein J^{D} is F, Cl or methyl.

18. The method of any one of claims 1-15, wherein J^{D} is F.

19. The method of any one of claims 1-18, wherein J^{C} is H or F.

20. The method of any one of claims 1-18, wherein J^{C} is H.

21. The method of any one of claims 1-20, wherein the CNS disease is Alzheimer's disease.

22. The method of claim 21, wherein the Alzheimer's disease is mild to moderate Alzheimer's disease or moderate to severe Alzheimer's disease.

23. The method of any one of claims 1-20, wherein the CNS disease is cognitive impairment.

24. The method of any one of claims 1-20, wherein the CNS disease is dementia.

25. The method of any one of claims 1-20, wherein the CNS disease is subjective cognitive impairment (SCI).

26. The method of any one of claims 1-20, wherein the CNS disease is cognitive ageing.

27. The method of any one of claims 1-20, wherein the CNS disease is vascular dementia.

28. The method of any one of claims 1-20, wherein the CNS disease is mixed dementia.

29. The method of any one of claims 1-20, wherein the CNS disease is Parkinson's disease.

30. The method of any one of claims 1-20, wherein the CNS disease is mild cognitive impairment.

31. The method of any one of claims 1-20, wherein the CNS disease is traumatic (closed or open) penetrating head injuries, traumatic brain injury (TBI), nontraumatic stroke, aneurism, hypoxia, or other injuries to the brain.

32. The method of any one of claims 1-20, wherein the CNS disease is stroke.

33. The method of claim 32, wherein the CNS disease is ischemic stroke.

34. The method of any one of claims 1-33, wherein the method further comprising administering to the subject an additional therapeutic agent.
